SEQUENCE LISTING (16 sequences)

SEQ ID NO: 1 - protein, 117 aa, synthetic construct [sequence omitted]

SEQ ID NO: 2 - protein, 108 aa, synthetic construct [sequence omitted]

SEQ ID NO: 3 - protein, 5 aa, synthetic construct
  REGION 1..5: note = HCDR1
NYWIA

SEQ ID NO: 4 - protein, 17 aa, synthetic construct
  REGION 1..17: note = HCDR2
IIYPGDSDIRYSPSFQG

SEQ ID NO: 5 - protein, 8 aa, synthetic construct
  REGION 1..8: note = HCDR3
HDIEGFDY

SEQ ID NO: 6 - protein, 12 aa, synthetic construct
  REGION 1..12: note = LCDR1
RASQSVSSSFFA

SEQ ID NO: 7 - protein, 7 aa, synthetic construct
  REGION 1..7: note = LCDR2
GASSRAT

SEQ ID NO: 8 - protein, 9 aa, synthetic construct
  REGION 1..9: note = LCDR3
QQYDSSAIT

SEQ ID NO: 9 - protein, 107 aa, synthetic construct [sequence omitted]

SEQ ID NO: 10 - protein, 330 aa, synthetic construct
  REGION 1..330: note = Heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, synthetic construct
  REGION 1..447: note = Heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, synthetic construct
  REGION 1..215: note = Light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 80 aa, synthetic construct
  REGION 1..80: note = H15D10 - VH
EVQLVQSGAEVKKPGESLRISCKGSGYTFTNYWVAWVRQMPGKGLESMGIIYPGDSDTRYSPSFQGHVTISADKSISTAY

SEQ ID NO: 14 - protein, 108 aa, synthetic construct
  REGION 1..108: note = L8C3 - VL
DIQMTQSPSSLSASLGDRVTITCRASQNVGNYLNWYQQKPGKAPKLLIYRASNLASGVPSRFSGSGSGTDFTLTISSLQPEDFATYYCQQMEHAPPTFGQGTKVEIKR

SEQ ID NO: 15 - protein, 108 aa, synthetic construct
  REGION 1..108: note = L16C11 - VL
EIVLTQSPGTLSLSPGERATLSCRASQSVIGYYLAWYQQKPGQAPRLLIYSVSTLASGIPDRFSGSGSGTDFTLTISRLEPEDFAVYYCQQYYRFPITFGQGTKVEIK

SEQ ID NO: 16 - protein, 117 aa, synthetic construct
  REGION 1..117: note = H19B7 - VH
EVQLVQSGAEVKKPGESLRISCKGSGYTFTNYWMAWVRQMPGKGLESMGIIYPSDSDTRYSPSFQGHVTISADKSISTAYLQWSSLKASDTAMYYCARHDVEGYDYWGQGTLVTVSS

(19) United States
(12) Patent Application Publication
(10) Pub. No.: US 2025/0326850 A1
(43) Pub. Date: Oct. 23, 2025

(54) TREATMENT OF LUPUS

(71) Applicant: ASTRAZENECA AB, SODERTALJE (SE)

(72) Inventors: CATHARINA LINDHOLM, SODERTALJE (SE); RAJENDRA TUMMALA, WILMINGTON, DE (US); EMMANUELLE MAHO, CAMBRIDGE (GB)

(21) Appl. No.: 18/291,671

(22) PCT Filed: Jul. 26, 2022
(86) PCT No.: PCT/EP2022/070860

§ 371 Date: Jan. 24, 2024

Related U.S. Application Data
(60) Provisional application No. 63/225,974, filed on Jul. 27, 2021.
(60) Provisional application No. 63/254,663, filed on Oct. 12, 2021.

(51) Int. Cl. C07K 16/28; A61P 37/06

(52) U.S. Cl. CPC C07K 16/2866; A61P 37/06

(57) ABSTRACT

The disclosure relates to methods and compositions for the treatment of systemic lupous erythematous (SLE). Specifically, the disclosure relates to methods comprising administering to a subject a type I IFN receptor inhibitor.

### CROSS REFERENCE TO RELATED APPLICATIONS

This present application is a U.S. national stage application under 35 U.S.C. § 371 of International Application No. PCT/EP2022/070860, filed Jul. 26, 2022, which claims priority to U.S. Provisional Application No. 63/225,974, filed Jul. 27, 2021, 2021, and to U.S. Provisional Application No. 63/254,663, filed Oct. 12, 2021, 2021, the entire contents of which are incorporated herein by reference in their entirety.

### REFERENCE TO AN ELECTRONIC SEQUENCE LISTING

The instant application contains an electronic Sequence Listing that has been submitted electronically and is hereby incorporated by reference in its entirety. The sequence listing was created on Jul. 18, 2022, is named “IFNAR-776-WO-PCT.xml” and is 22,443 bytes in size.

### 1. BACKGROUND

### 1.1. Systemic Lupus Erythematosus (SLE)

Systemic lupus erythematosus (SLE) is a chronic, multisystemic, disabling autoimmune rheumatic disease of unknown etiology. There is substantial unmet medical need in the treatment of SLE, particularly in subjects with moderate or severe disease. Long-term prognosis remains poor for many subjects.

A significant problem associated with the treatment of SLE, is the heterogeneous clinical manifestations of SLE¹. Any organ may be affected in SLE, with the skin, joints, and kidneys being the most commonly involved²⁻⁴. Incomplete disease control leads to progressive organ damage, poor quality of life, and increased mortality, with approximately half of all patients with SLE developing organ damage within 10 years of diagnosis⁵⁻⁶. There remains the need for a medical intervention that improves SLE disease activity across multiple systems.

Clinical manifestations of SLE include, but are not limited to, constitutional symptoms, alopecia, rashes, serositis, arthritis, nephritis, vasculitis, lymphadenopathy, splenomegaly, haemolytic anaemia, cognitive dysfunction and other nervous system involvement. Increased hospitalisations and side effects of medications including chronic oral corticosteroids (OCS) and other immunosuppressive treatments add to disease burden in SLE⁷⁻⁹.

All of the therapies currently used for the treatment of SLE have well known adverse effect profiles and there is a medical need to identify new targeted therapies, particularly agents that may reduce the requirement for corticosteroids and cytotoxic agents. There has been only 1 new treatment (belimumab) for SLE approved by the US Food and Drug Administration (FDA) and European Medicines Agency (EMA) in the approximately 50 years since hydroxychloroquine was approved for use in discoid lupus and SLE. However, belimumab is not approved everywhere, and the uptake has been modest. Many agents currently used to treat SLE, such as azathioprine, cyclophosphamide, and mycophenolate mofetil/mycophenolic acid, have not been approved for the disease. Furthermore, these drugs all have well-documented safety issues and are not effective in all patients for all manifestations of lupus. Antimalarial agents (e.g. hydroxychloroquine) and corticosteroids may be used to control arthralgia, arthritis, and rashes. Other treatments include nonsteroidal anti-inflammatory drugs (NSAIDs); analgesics for fever, arthralgia, and arthritis; and topical sunscreens to minimize photosensitivity. It is often difficult to taper subjects with moderate or severe disease completely off corticosteroids, which cause long-term morbidity and may contribute to early cardiovascular mortality⁸⁻¹⁰. Even small daily doses of 5 to 10 mg prednisone used long-term carry increased risks of side effects such as cataracts, osteoporosis, and coronary artery disease⁸.

The clinical development of a new drug is a lengthy and costly process with low odds of success. For molecules that enter clinical development, less than 10% will eventually be approved by health regulatory authorities¹¹. Furthermore, the early clinical development of biotherapeutics is much lengthier than for small molecules.

Phase II trials are conducted in a small number of volunteers who have the disease of interest. They are designed to test safety, pharmacokinetics, and pharmacodynamics. A phase II trial may offer preliminary evidence of drug efficacy. However, the small number of participants and primary safety concerns within a phase II trial usually limit its power to establish efficacy. A Phase III trial is required to demonstrate the efficacy and safety of a clinical candidate. Critically, many clinical candidates that have shown promise at Phase II fail at Phase III. More than 90% of novel therapeutics entering Phase I trials fail during clinical development, primarily because of failure in efficacy or safety. The probability of success at phase III, following successful Phase II, is less than 50%¹².

The process of drug development is particularly difficult for SLE. This is because SLE is an especially complex and poorly understood disease. Not only is our understanding of the genetics of SLE rudimentary, but our insight into pathogenesis of most of the clinical manifestations are still relatively limited compared to other disease.

The complexity of SLE presents those wishing to develop new therapeutics with the problem of a patient population with extensive inhomogeneity¹³. This makes protocol design for clinical trials in SLE even more difficult, for example, as regards to the choice of inclusion criteria and primary and secondary endpoints. It is further difficult to predict the disease course in each patient. This inevitably increases the background noise that reduces the statistical power of a trial. A high placebo response rate limits the range in which the tested new drug can show an efficacy signal, making clinical trials even more difficult to conduct and interpret.

The difficulty in developing effective therapeutics for SLE leads to an even higher failure rate of therapeutics in this area in clinical trials, compared to therapeutics for other indications. The development of novel therapeutics for the treatment of SLE has thus proved extremely difficult. There are many examples of clinical candidates that showed promise at Phase II but failed to show efficacy and/or safety in subsequent Phase or Phase III trials.

There remains an unmet clinical need in SLE particularly in patients resistant to conventional immunosuppressive therapies and immunomodulators. There is also a subset of SLE patients whose disease remains refractory to existing treatments (refractory disease, RD). Morbidity and mortality from active SLE remains high in such patients, and the search for safe and effective therapies to treat them is ongoing

### 1.2. Steroids

Glucocorticoids remain the mainstay treatment for SLE with doses varying depending on severity of disease manifestation. There is no “safe” dose of oral glucocorticoids in relation to the risk for development of glucocorticoid-induced damage such as cataracts, osteoporosis and coronary artery disease, and whereas higher glucocorticoid-exposure has been shown to be associated with increased overall damage accrual, fairly low to moderate doses can also be related to increased damage.

Glucocorticoids are the most commonly used therapy for patients with SLE owing to their immunosuppressant and anti-inflammatory properties, which reduce disease activity and prevent flares. Up to 80% of patients with SLE are exposed to glucocorticoids, with the majority being treated long-term. Although it may provide short-term efficacy, the frequent or maintenance use of oral glucocorticoid therapy carries a significant burden of toxicity that can independently contribute to morbidity and mortality and can adversely affect health-related quality of life. Therefore, novel, effective, and long-term treatments for SLE are needed to both reduce overall disease activity and glucocorticoid use.

### 1.3. Type I IFN and Anifrolumab

Anifrolumab (MEDI-546) is a human immunoglobulin G1 kappa (IgG1κ) monoclonal antibody (mAb) directed against subunit 1 of the type I interferon receptor (IFNAR1). It is composed of 2 identical light chains and 2 identical heavy chains, with an overall molecular weight of approximately 148 kDa. Anifrolumab inhibits binding of type I IFN to type I interferon receptor (IFNAR) and inhibits the biologic activity of all type I IFNs.

Type I interferons (IFNs) are cytokines that have been implicated in SLE pathogenesis based on the finding of increased IFN-stimulated gene expression in most patients with SLE. In the phase 3 TULIP-2 trial of anifrolumab in patients with moderate to severe SLE, treatment response (assessed using British Isles Lupus Assessment Group [BILAG]-based Composite Lupus Assessment [BICLA]) was achieved by significantly more patients receiving anifrolumab compared with placebo at Week 5214. Similar results with this composite endpoint were observed in the phase 2 MUSE and phase 3 TULIP-1 trials 15.16

### 1.4. Conclusion

There is a huge unmet need for an SLE therapy with a better efficacy and safety profile the currently available therapies^{17,18}. As described above, a large number and broad range of different biologics have been proposed and subjected to clinical trials, but these trials have failed to meet clinical meaningful endpoints in pivotal studies. Initial promise at Phase II of many proposed therapeutics was not translated into significant and meaningful clinical effect in subsequent pivotal Phase III clinical trials. Furthermore, there is a need for an SLE therapy that is efficacious across multiple organ domains. Furthermore, even approved treats for SLE do not permit steroid tapering in many patients. There is further a subset of patients with refractory disease, i.e. having moderate to severe SLE despite treatment with standard therapies. Thus, there remains the need for safe and effective treatment of SLE that has proven clinical benefit, for example in a phase III double-blind, randomized, placebo controlled trial¹⁹.

The present invention solves one or more of the above-mentioned problems.

### 2. Summary

The present invention relates to a method of treating systemic lupus erythematous (SLE) in a subject in need thereof, the method comprising administering a type I IFN receptor (IFNAR1) inhibitor to the subject, wherein the method reduces SLE activity in the subject compared to the SLE activity in the subject before treatment with the IFNAR1 inhibitor, and wherein the subject has received prior treatment with one or more immunomodulators before administration of the IFNAR1 inhibitor.

The invention also relates to a method of identifying a subject as suitable for treatment with an IFNAR1 inhibitor, the method comprising identifying the subject as having received prior treatment with one or more immunomodulators before administration of the IFNAR1 inhibitor, and administering the IFNAR1 inhibitor to the subject.

The invention also relates to a method of treating SLE in a subject in need thereof, the method comprising administering a IFNAR1 inhibitor to the subject, wherein the method reduces SLE activity in the subject compared to the SLE activity in the subject before treatment with the IFNAR1 inhibitor, and wherein the subject has severe, established and/or refractory SLE.

The invention is supported inter alia by data, presented herein for the first time, from two phase III, multicenter, multinational, randomized, double-blind, placebo-controlled clinical trials (NCT02446899 and NCT02962960) demonstrating that an IFNAR1 inhibitor (anifrolumab) treats SLE in patients that have been previously treated with one or more immunomodulator biologics, wherein the SLE disease in the subject has not been controlled. Treatment with anifrolumab is more effective in these patients than in biologic naïve patients. The invention is also supported by data, presented herein for the first time, demonstrating that an IFNAR1 inhibitor treats SLE in subjects who have moderate to severe SLE despite standard therapy (i.e. refractory disease).

### 3. BRIEF DESCRIPTION OF FIGURES

FIG. 1: IFN scores distribution

FIGS. 2A and 2B: Previous use of biologic immunomodulators

FIG. 2A: Antineoplastic and immunomodulating agents. FIG. 2A: Musculo-skeletal system. Percentages are based on all patients in the full analysis set within the respective study and treatment group. All medication is coded using WHO-DD version 2019SEP01 B3. Previous use: the end date is prior to investigational product dose (anifrolumab) administration on Day 1. Biologic immunomodulators are identified using (ATC level 4 and preferred term: L04AA=Selective immunosuppressants except preferred terms MYCOPHENOLATE MOFETIL, MYCOPHENOLATE MOFETIL HYDROCHLORIDE, MYCOPHENOLATE SODIUM, MYCOPHENOLIC ACID, APREMILAST, BARICITINIB, IGURATIMOD and LEFLUNOMIDE) or (ATC level 3: L01X=OTHER ANTINEOPLASTIC AGENTS) or (preferred terms: ADALIMUMAB, BLISIBIMOD, ETANERCEPT, INFLIXIMAB, RONTALIZUMAB, SIFALIMUMAB, TABALUMAB and USTENKINUMAB). Phase III pool includes study 04 (D3461C00004) and study 05 (D3461C00005) (excluding the 150 mg group from study 05). ATC: Anatomical Therapeutic Chemical; N: Number of patients in treatment group; WHO-DD World Health Organization Drug Dictionary.

FIG. 3: Baseline demongraphics, disease characteristics, and SLE treatments in biologic-experienced and biologic-naïve patients with SLE pooled from the TULIP-1 and TULIP-2 trials

Baseline demographics, disease characteristics, and non-biologic SLE treatments were generally similar between groups.

FIG. 4: Forest plot of efficacy endpoints in the biologic-experienced and biologic-naïve patients with SLE in data pooled from the TULIP-1 and TULIP-2 trials

Anifrolumab was associated with greater treatment differences over placebo (Δ) in biologic-experienced vs biologic-naïve patients across endpoints, including BICLA response (Δ=19.4 vs Δ=16.6), SRI(4) response (Δ=25.3 vs Δ=9.1), and oral GC tapers (Δ=24.7 vs Δ=17.5).

FIGS. 5A and 5B: BICLA response rate at Week 52 by previous use of abatacept, stratified Cochran-Mantel-Haenszel approach

FIG. 5A: Previous use of abatacept. FIG. 5B: No previous use of abatacept. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 6A and 6B: BICLA response rate at Week 52 by previous use of belimumab, stratified Cochran-Mantel-Haenszel approach

FIG. 6A: Previous use of BELIMUMAB. FIG. 6B: No previous use of belimumab. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 7A and 7B: BICLA response rate at Week 52 by previous use of epratuzumab, stratified Cochran-Mantel-Haenszel approach

FIG. 7A: Previous use of epratuzumab. FIG. 7B: No previous use of epratuzumab. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 8A and 8B: BICLA response rate at Week 52 by previous use of rituximab, stratified Cochran-Mantel-Haenszel approach

FIG. 8A: Previous use of rituximab. FIG. 8B: No previous use of rituximab. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 9A and 9B: BICLA response rate at Week 52 by previous use of sifalimumab, stratified Cochran-Mantel-Haenszel approach

FIG. 9A: Previous use of sifalimumab. FIG. 9B: No previous use of sifalimumab. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 10A and 10B: BICLA response rate at Week 52 by previous use of tabalumab, stratified Cochran-Mantel-Haenszel approach

FIG. 10A: Previous use of tabalumab. FIG. 10B: No previous use of tabalumab. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1.

FIGS. 11A and 11B: BICLA response rate at Week 52 by previous use of TNF inhibitors, stratified Cochran-Mantel-Haenszel approach

FIG. 11A: Previous use of TNF inhibitors. FIG. 11B: No previous use of TNF inhibitors. Baseline is defined as the last measurement prior to randomization and investigational product does administration on Day 1. Phase III pool includes study 04 (D3461C00004) and study 05 (D3461C00005) (excluding the 150 mg group from study 05). BICLA response is defined as reduction of all baseline BILAG-2004 A and B scores and no worsening in other organ systems, no worsening from baseline in SLEDAI-2K, and no increase in >=0.030 points on a 3 point PGA VAS from baseline. Subjects treated with restricted medication beyond protocol allowed threshold, and those who discontinued investigational product (anifrolumab), are regarded as non-responders. The responder/non-responder rates (percentages, the difference in estimates and associated 95% CI are calculated using a stratified CMH approach, with stratification factors SLEDAI-2K score at screening, Day 1 OCS dose and type I IFN gene signature test result at screening. In the pooled analysis an additional stratification factor is added for study (study 04 vs study 05). The nominal p-values presented are based on this CMH model. Previous use: the end date of therapy is prior to investigational product dose administration on Day 1. TNF inhibitors are identified using preferred terms: adalimumab, etanercept, infliximab.

FIG. 12: Safety in biologic-experienced and biologic-naïve patients with SLE in data pooled from the TULIP-1 and TULIP-2 trials

Incidence of serious adverse events was higher in biologic-experienced vs biologic-naïve patients with anifrolumab and placebo.

FIGS. 13A and 13B: Mean anifrolumab serum concentration-time profiles

FIG. 13A: Study MI-CP180 in SSc—Mean anifrolumab serum concentration-time profiles following a single IV dose. Data represent+/−SD. Mean data below LLOQ are not plotted. IV, intravenous; LLOQ, lower limit of quantification; MEDI 546, anifrolumab; n, number of patients in a subgroup; SSc, systemic sclerosis. FIG. 13B: Study 06 in healthy volunteers-Mean anifrolumab serum concentration-time profiles following a single SC and IV dose. Samples with actual collection time deviating from nominal collection time by >10% were excluded from the mean. IV, intravenous; N, number of subjects; SC, subcutaneous.

FIG. 14A-14C: Study 08 study design and results

FIG. 14A: Study design for phase II of SC anifrolumab in SLE patients. Study 08 (NCT02962960) evaluated the effect of two anifrolumab doses every other week. FIG. 14B: Mean serum concentration of anifrolumab over time. FIG. 14C: Anifrolumab neutralization of the type I IFN gene signature

FIGS. 15A and 15B: Computed median AUC Ratios (SC/IV)

FIG. 15A: Computed median AUC Ratio (SC/IV) between weeks 0-52 for various SC doses. The computed median AUC Ratio (SC/IV), based on the estimated bioavailability from Study 06, between weeks 0-52, where the subcutaneous dose is either 75 mg (+sign), 90 mg (empty squares), 105 mg (circles), 120 mg (triangles), or 135 mg (filled squares). The subcutaneous dose here is administered once every 7 days (QW); the IV dose is administered once every 4 weeks (Q4W) at a dose of 300 mg. Based on the AUC, both 90 and 105 mg SC QW appear similar to 300 mg IV. FIG. 15B: Computed median AUC ratio (SC/IV) for 90 mg and 105 mg SC QW. The computed median AUC Ratio (SC/IV), based on the estimated bioavailability ˜7% lower than the bioavailability calculated from Study 06, between weeks 0-52, where the subcutaneous dose is either 90 mg SC QW or 105 mg SC.

FIGS. 16A and 16B: Anifrolumab concentration over time at different doses

FIG. 16A: A plot showing (computed) trough concentrations of plasma anifrolumab in a patient administered either (i) 105 mg of anifrolumab subcutaneously, once every 7 days (straight line); (ii) 300 mg anifrolumab intravenously, once every 4 weeks (lower dotted line); (ii) 1000 mg anifrolumab intravenously, once every 4 weeks (upper dotted line). Shaded area represents the area between 5th and 95th percentiles of the 300 mg IV Q4W dose. FIG. 16B: Anifrolumab trough concentration in IFNGS high SLE subjects. Computed trough concentrations of anifrolumab in IFNGS high patients plasma after administration as follows: (i) 300 mg IV Q4W; (ii) 90 mg SC QW; (iii) 105 mg SC QW; (iv) 135 mg SC QW; (v) 1000 mg IV Q4W. SC=subcutaneous. Based on trough, both 90 and 105 mg SC QW were projected to have higher PD suppressions than 300 mg IV.

FIGS. 17A and 17B: Positive Exposure-BICLA relationship observed in TULIP 1 & TULIP 2 in IFNGS high patients

FIG. 17A: TULIP I, for placebo, 150 mg and 300 mg anifrolumab. FIG. 17B: TULIP II, for placebo and 300 mg.

FIGS. 18A and 18B: BICLA dose response

FIG. 18A: Dose response curve, for probability of meeting BICLA response criteria (in IFNGS high patients) versus anifrolumab Cave over 52 weeks, showing the predicted mean (grey line) and 95% confidence interval (CI) (dashed area). Patients are grouped by dose (150 mg, n=62; 300 mg, n=242; and 1000 mg). FIG. 18B: Predicted PK and efficacy for different SC doses. The probability of meeting BICLA (in IFNGS high patients) for weekly subcutaneous doses starting from 105 mg, and up to 150 mg. Assumptions for generating the data include no dose delays/interruptions.

FIGS. 19A and 19B: C_{troughs} following injection at thigh compared to injection at abdomen FIG. 19A: 150 mg SC Q2W. FIG. 19B: 300 mg SC Q2W

FIG. 20: Exposure prediction based on 81-87% bioavailability and preliminary PK modelling

Anifrolumab Cₐᵥₑ medium ratio predicted for 90-150 mg SC QW to 300 mg Q4W, based on PK preliminary modelling and bioavailability assumptions.

FIG. 21A-21C: Anifrolumab Cₐᵥₑ over 52 weeks in IFNGS high patients for different SC and IV doses

FIG. 21A: 105 mg SC QW. FIG. 21B: 120 mg SC QW. FIG. 21C: Overlap with 1000 mg IV Q4W.

FIGS. 22A and 22B: Cₐᵥₑ median ratio SC QW to 300 mg IV Q4W FIG. 22A: 81% bioavailability assumed. FIG. 22B: 70% bioavailability assumed.

FIG. 23: Average anifrolumab concentration versus herpes zoster incidence

The incidence of Herpes Zoster (%) in patients in the Study 1013 receiving placebo, 300 mg IV anifrolumab or 1000 mg IV anifrolumab.

FIG. 24: Baseline demographics, SLE disease characteristics, and treatments in data pooled from TULI P-1 and TULIP-2 trials

FIG. 24: Baseline demographics in data pooled from TULIP-1 and TULIP-2, baseline disease characteristics in data pooled from TULIP-1 and TULIP-2, and baseline SLE treatments in data pooled from TULIP-1 and TULIP-2. Patients with established vs recent-onset disease were more likely to be IFN gene signature (IFNGS) high (83.5% vs 78.8%), anti-dsDNA antibody-positive (45.6% vs 38.6%), complement4 (C4) low (24.7% vs 16.7%), have≥1 BILAG-2004A item (49.7% vs 43.9%), and have a higher mean global SDI score (0.7 vs 0.1). Patients with established vs recent-onset disease were more likely to be receiving oral glucocorticoids (GC) (83.2% vs 76.5%), and/or immunosuppressants (49.8% vs 40.9%), but not antimalarials (69.5% vs 78.0%). C, complement; CLASI, Cutaneous Lupus Erythematosus Disease Area and Severity Index; PGA, Physician's Global Assessment; SD, standard deviation; SDI, SLICC/ACR Damage Index. IST, immunosuppressants. a Immunosuppressants: azathioprine, methotrexate, mycophenolate mofetil, mycophenolic acid, and mizoribine.

FIGS. 25A and 25B: Anifrolumab treatment of established disease

FIG. 25A: Proportion of patients with a BICLA response at week 52 in the TULIP-1 and TULIP-2 trials. FIG. 25B: BILAG-2004 organ involvement scores at baseline by disease duration: Patients are based on all patients within the respective disease-duration group, who were either treated with anifrolumab 300 mg or placebo. Organ domains (gastrointestinal, ophthalmic, hematologic) were not included on the graph, as, 1% of patients in either disease duration group had a BILAG-2004 A or B score. Phase 3 pool includes TULIP-1 and TULIP-2 (excluding anifrolumab 150 mg group from TULIP-1).

FIG. 26: Forest plot of BICLA response according to baseline standard therapy in patients with SLE in TULIP-1 and TULIP-2

BICLA, BILAG-based Composite Lupus Assessment; CI, confidence interval; GC, glucocorticoid; IFNGS, interferon gene signature; n, number of responders; N, number of patients in the group; PGA, Physicians' Global Assessment. A BICLA response required: Reduction of all baseline BILAG-2004 A or ≥2 new BILAG-2004 B0; no increase in SLEDAI-2K score from baseline; no increase in ≥0.3 points in PGA score from baseline; no use of restricted medications beyond protocol-allowed thresholds, and no discontinuation of investigational product. The response rates, the differences in response rates, and associated 95% Cis were calculated using a stratified Cochran-Mantel-Haenszel method with stratification factors of SLEDAI-2K score at screening (<10 vs ≥10), baseline oral GC dosage (<10 vs ≥10 mg/day prednisone or equivalent), IFNGS status (high vs low), and study. ^{a}Prednisone or equivalent. ^{b}Immunosuppressants were ≥1 or: azathioprine, methotrexate, mycophenolate, or mycophenolic acid.

FIG. 27A-27C. Delivery device

Anifrolumab is administered by an injection device [1] [9] such as a prefilled syringe (PFS) (FIG. 27A) or an autoinjector (AI) (FIG. 27B and FIG. 27C).

FIG. 28A-28C. Autoinjector

The autoinjector for administering anifrolumab of the functional variant thereof in exploded view (FIG. 28A), assembled (FIG. 28B) and filled with drug substance (FIG. 28C).

FIG. 29A-29D. Accessorized pre-filled syringe

The accessorized pre-filled syringe (APFS) for anifrolumab of the functional variant thereof. The primary tube is shown in assembled form (FIG. 29A) and in exploded view (FIG. 29B). The APFS with its additional components is shown in assembled form (FIG. 29C) and in exploded view (FIG. 29D).

FIG. 30. Packaging for the delivery device

### 4. DETAILED DESCRIPTION

### 4.1. Method of treating systemic lupus erythematous (SLE)

The invention relates to a method of treating systemic lupus erythematous (SLE) in a subject in need thereof, the method comprising administering a type I IFN receptor (IFNAR1) inhibitor to the subject, wherein the method reduces SLE activity in the subject compared to the SLE activity in the subject before treatment with the IFNAR1 inhibitor, and wherein the subject has received prior treatment with one or more immunomodulators before administration of the IFNAR1 inhibitor. SLE disease activity refers to pathology associated with SLE disease, for example as listed in Systemic Lupus Erythematosus Disease Activity Index 2000 (SLEDAI 2K) ²⁰, Systemic Lupus International Collaborating Clinics/American College of Rheumatology Damage Index²⁰, Cutaneous Lupus Erythematosus Disease Area and Severity Index (CLASI)²¹, British Isles Lupus Assessment Group-2004 index^{22,23}, or ACR Classification Criteria for SLE (ACR 199724 and/or EULAR/ACR 201925). Reducing SLE disease activity in the subject may comprise a BILAG-Based Composite Lupus Assessment (BICLA) response in the subject. Reducing SLE disease activity in the subject may comprise a SRI(4) response in the subject. Reducing SLE disease activity in the subject may comprise a reduced annualized flare rate in the subject.

The ability of the IFNAR1 inhibitor to reduce SLE disease activity in a subject refractive to treatment with the one more immunomodulators or in a subject that has relapsed during or following treatment with the one or more immunomodulators, may have been demonstrated in a phase III clinical trial, optionally the phase III clinical trial is a multicentre, multinational, randomised, double-blind, placebo-controlled study.

### 4.2. Method of Identifying a Subject

The invention also relates to a method of identifying a subject as suitable for treatment with an IFNAR1 inhibitor, the method comprising identifying the subject as having received prior treatment with one or more immunomodulators before administration of the IFNAR1 inhibitor, and administering the IFNAR1 inhibitor to the subject. The method may comprise identifying the subject as having severe, refractory and/or established SLE disease activity prior to treatment with the IFNAR1 inhibitor. Severe SLE may be defined as a SLEDAI-2K score ≥10. Severe SLE may be defined as ≥1 A BLIAG-2004 organ score. Severe SLE may be defined as a CLASI activity score of ≥10. Severe SLE may be defined as ≥6 swollen and ≥6 tender joints. Severe SLE may be defined as a SDI (SLICC/ACR Damage Index) global score of ≥1. Severe SLE may be defined as ≥1 A BLIAG-2004 organ score, a CLASI activity score of ≥10, ≥6 swollen and ≥6 tender joints and/or a SDI global score of ≥1. Refractory SLE may be defined as severe SLE despite standard of care treatment with oral glucocorticoids, antimalarials, NSAIDs and/or one or more immunosuppressants. The one or more immunosuppressants may include azathioprine, methotrexate, mycophenolate.

The method may comprise identifying the subject as having been diagnosed with SLE at least 2 years before treatment with the IFNAR1 inhibitor. The method may comprise identifying the subject as being IFN gene signature high subject, anti-dsDNA antibody positive, complement 4 (C4) low, having ≥1 BILAG-2004 A item and/or having a higher mean global SDI score. Low complement may be defined as less than about 0.1 g/L C4 in the blood and/or less than about 0.9 g/L C3 in the blood.

### 4.3. Immunomodulators

The one or more immunomodulators may comprise a biologic. The one or more immunomodulators may comprise abatacept, atacicept, belimumab, epratuzumab, rituximab, tabalumab, sifalimumab, adalimumab and/or infliximab. The one or more immunomodulators may comprise a CTLA-4 fusion protein. The CTLA-4 fusion protein may be abatacept or a functional equivalent thereof. The CTLA-4 fusion protein may be abatacept or a functional equivalent thereof. The one or more immunomodulators may comprise an anti-BAFF antibody. The anti-BAFF antibody may be belimumab or a functional equivalent thereof. The one or more immunomodulators may comprise an anti-CD20 antibody. The anti-CD20 antibody may be rituximab or a functional equivalent thereof. The one or more immunomodulators comprise an anti-type I IFN antibody. The anti-type I IFN antibody may be sifalimumab or a functional equivalent thereof. The one or more immunomodulators comprise belimumab and rituximab. The one or more immunomodulators may not comprise an IFNAR1 inhibitor.

The subject may have received prior treatment with immunomodulatory at least 3 months prior to treatment with the IFNAR1 inhibitor. The subject may have received prior treatment with immunomodulatory at least 2 months prior to treatment with the IFNAR1 inhibitor. The subject may have received prior treatment with immunomodulatory at a month prior to treatment with the IFNAR1 inhibitor.

### 4.4. IFNAR1 Inhibitor

A “type I interferon receptor inhibitor” refers to a molecule that is antagonistic for the receptor of type I interferon ligands such as interferon-a and interferon-B. Such inhibitors, subsequent to administration to a patient, preferably provide a reduction in the expression of at least 1 (preferably at least 4) pharmacodynamic (PD) marker genes selected from the group consisting of IFI6, RSAD2, IFI44, IFI44L, IFI27, MX1, IFIT1, HERC5, ISG15, LAMP3, OAS3, OAS1, EPST1, IFIT3, LY6E, OAS2, PLSCR1, SIGLECI, USP18, RTP4, and DNAPTP6. The at least 4 genes may suitably be IFI27, IFI44, IFI44L, and RSAD2. The “type I interferon receptor” is preferably interferon-a/B receptor (IFNAR).

For example, the type I interferon receptor inhibitor may be an antibody or antigen-binding fragment thereof that inhibits type I IFN activity (by inhibiting the receptor). An example of a suitable antibody or antigen-binding fragment thereof (that inhibits type I IFN activity) is an interferon-a/B receptor (IFNAR) antagonist. The type I interferon receptor inhibitor may be an antibody or antigen-binding fragment thereof that inhibits type I IFN activity. Additionally or alternatively, the type I interferon receptor inhibitor may be a small molecule inhibitor of a type I interferon receptor (e.g. for pharmacological inhibition of type I interferon receptor activity).

The IFNAR1 inhibitor may be a human monoclonal antibody specific for IFNAR1. The IFNAR1 inhibitor may be a modified IgG1 class human monoclonal antibody specific for IFNAR1.

The antibody may comprise a heavy chain variable region complementarity determining region 1 (HCDR1) comprising the amino acid sequence of SEQ ID NO: 3. The antibody may comprise a heavy chain variable region complementarity determining region 2 (HCDR2) comprising the amino acid sequence of SEQ ID NO: 4. The antibody may comprise a heavy chain variable region complementarity determining region 3 (HCDR3) comprising the amino acid sequence of SEQ ID NO: 5. The antibody may comprise a light chain variable region complementarity determining region 1 (LCDR1) comprising the amino acid sequence SEQ ID NO: The antibody may comprise a light chain variable region complementarity determining region 2 (LCDR2) comprising the amino acid sequence SEQ ID NO: 7. The antibody may comprise a light chain variable region complementarity determining region 3 (LCDR3) comprising the amino acid sequence SEQ ID NO: 8.

The antibody may comprise a human heavy chain variable region comprising the amino acid sequence of SEQ ID NO: 1. The antibody may comprise a human light chain variable region comprising the amino acid sequence of SEQ ID NO: 2. The antibody may comprise a human light chain constant region comprising the amino acid sequence of SEQ ID NO: 9. The antibody may comprise a human heavy chain constant region comprising the amino acid sequence of SEQ ID NO: 10. The antibody may comprise in the Fc region an amino acid substitution of L234F, as numbered by the EU index as set forth in Kabat and wherein said antibody exhibits reduced affinity for at least one Fc ligand compared to an unmodified antibody. The antibody may comprise a human heavy chain comprising the amino acid sequence of SEQ ID NO: 11. The antibody may comprise a human light chain comprising the amino acid sequence of SEQ ID NO: 12.

The antibody may comprise: (a) a heavy chain variable region complementarity determining region 1 (HCDR1) comprising the amino acid sequence of SEQ ID NO: 3; (b) a heavy chain variable region complementarity determining region 2 (HCDR2) comprising the amino acid sequence of SEQ ID NO: 4; c) a heavy chain variable region complementarity determining region 3 (HCDR3) comprising the amino acid sequence of SEQ ID NO: 5; (d) a light chain variable region complementarity determining region 1 (LCDR1) comprising the amino acid sequence SEQ ID NO: 6; (b) a light chain variable region complementarity determining region 2 (LCDR2) comprising the amino acid sequence SEQ ID NO: 7; c) a light chain variable region complementarity determining region 3 (LCDR3) comprising the amino acid sequence SEQ ID NO: 8.

The antibody may comprise (a) a human heavy chain comprising the amino acid sequence of SEQ ID NO: 11; and (b) a human light chain comprising the amino acid sequence of SEQ ID NO: 12.

The IFNAR1 inhibitor may be anifrolumab or a functional variant thereof.

### 4.5. Doses and Methods of Administration

The method may comprise administering an intravenous dose of anifrolumab or the functional variant thereof to the subject. The intravenous dose may be ≥300 mg anifrolumab or the functional variant thereof. The intravenous dose may be $1000 mg. The intravenous dose may be about 300 mg, about 900 mg or about 1000 mg. The intravenous dose may be administered every four weeks (Q4W).

The method may comprise administering a subcutaneous dose of anifrolumab or the functional variant thereof. The subcutaneous dose may be >105 mg and <150 mg anifrolumab or the functional variant thereof. The subcutaneous dose may be ≤135 mg anifrolumab or the functional variant thereof. The subcutaneous dose may be about 120 mg. The subcutaneous dose may be administered in a single administration step. The subcutaneous dose may be administered at intervals of 6-8 days. The subcutaneous dose may be administered once per week. The subcutaneous dose may have a volume of about 0.5 to about 1 m. The subcutaneous dose may have a volume of about 0.8 ml.

The subject may be a type I interferon stimulated gene signature (IFNGS)-test high patient pre-treatment. The method may comprise identifying the subject as IFNGS-test high patient pre-treatment.

Many patients with SLE receive corticosteroids (glucocorticoids, oral corticosteroids, OCS). However, corticosteroids are associated with organ damage. Anifrolumab permits tapering of the corticosteroids (glucocorticoids) in SLE patients (steroid sparing). The method of treatment or method may comprise administering a corticosteroid to the subject, optionally wherein the corticosteroid is an oral corticosteroid. The method may comprise tapering dose of corticosteroids administered to the subject (steroid sparing). The method may comprise administering a first dose of the corticosteroid and subsequently administering a second dose of the corticosteroid, wherein the second dose of the corticosteroid is lower than the first dose of the corticosteroid. The second dose of the corticosteroid may be about a 7.5 mg prednisone-equivalent dose or less. The second dose of the corticosteroid may be a 5 mg prednisone-equivalent dose or less. The method or method of treatment may comprise administrating the second dose of the corticosteroid once per day. The first dose of the corticosteroid may be about a 10 mg prednisone-equivalent dose. The method may comprise tapering the dose of corticosteroid administered to the patient from 10 mg or more per day to less than 10 mg per day. The method or method of treatment may comprise administering the second dose of the corticosteroid once per day. The method may permit administration of a reduced dose of corticosteroids that is sustained for weeks. The second dose of the corticosteroid may be administered for at least 24 weeks. The second dose of the corticosteroid may be administered for at least 28 weeks.

### 4.6. Steroid Sparing

The method may comprise steroid sparing in the subject, wherein the dose of the steroid administered to the subject is tapered from a pre-sparing dose at baseline to a post-sparing dose. The post-sparing dose may be $7.5 mg/day prednisone or prednisone equivalent dose. The pre-sparing dose may be 20 mg/day prednisone or prednisone equivalent dose. The steroid may comprise a glucocorticoid. The steroid may comprise an oral glucocorticoid. The steroid may be selected from the group consisting of hydrocortisone, mometasone, fluticasone, fluocinolone acetonide, fluocinolone, flurandrenolone acetonide, ciclesonide, budesonide, beclomethasone, deflazacort, flunisolide, beclomethasone dipropionate, betamethasone, betamethasone valerate, methylprednisolone, dexamethasone, prednisolone, cortisol, triamcinolone, clobetasol, clobetasol propionate, clobetasol butyrate, cortisone, corticosterone, clocortolone, dihydroxycortisone, alclometasone, amcinonide, diflucortolone valerate, flucortolone, fluprednidene, fluandrenolone, fluorometholone, halcinonide, halobetasol, desonide, diflorasone, flurandrenolide, fluocinonide, prednicarbate, desoximetasone, fluprednisolone, prednisone, azelastine, dexamethasone 21-phosphate, fludrocortisone, flumethasone, fluocinonide, halopredone, hydrocortisone 17-valerate, hydrocortisone 17-butyrate, hydrocortisone 21-acetate, prednisolone, prednisolone 21-phosphate, clobetasol propionate, triamcinolone acetonide, or a mixture thereof. The steroid may be prednisone.

### 4.7. Unit Dose

The invention also relates to a unit dose for use in the methods of the invention, wherein the unit dose comprises >105 mg and ≤150 mg anifrolumab or a functional variant thereof.

The unit dose may comprise ≤135 mg (i.e. 135 mg or less) anifrolumab or the functional variant thereof. The unit dose may comprise about 120 mg anifrolumab or the functional variant thereof. The unit dose may comprise 120 mg anifrolumab or the functional variant thereof. The unit dose may consist essentially of >105 mg and <150 mg anifrolumab or the functional variant thereof. The unit dose may consist essentially of ≤135 mg anifrolumab or the functional variant thereof. The unit dose may consist essentially of about 120 mg anifrolumab or the or the functional variant thereof. The concentration of anifrolumab or the functional variant thereof in the unit dose may be about 150 mg/ml. The volume of the unit dose may be less than 1 ml. The dose or unit dose may have a volume of about 0.5 to about 1 ml. The concentration of the unit dose may be about 0.8 ml. The volume of the unit dose may be 0.8 ml. The unit dose may comprise a formulation of about 150 to 200 mg/ml anifrolumab or the functional variant thereof, about 25 to 150 mM of lysine sale and an uncharged excipient. The unit dose may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

In another aspect the invention relates to a method of treating SLE in a subject, the method comprising subcutaneously administering a dose of anifrolumab or a functional variant thereof, wherein administering the dose every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is more than the plasma concentration provided by intravenous administration of 300 mg of anifrolumab or the functional variant thereof every 4 weeks. Administering the dose every week may provide a plasma concentration in the subject that is at least equivalent to the plasma concentration provided by intravenous administration of 400 mg of anifrolumab or the functional variant thereof every 4 weeks. The dose may be administered in a single-administration step. The dose administered to the subject may be <150 mg (i.e. less than 150 mg) anifrolumab or the functional variant thereof. The dose administered to the subject may be >105 mg (i.e. more than 105 mg) anifrolumab or the functional variant thereof. The dose of administered to the subject may be ≤135 mg (i.e. 135 mg or less) anifrolumab or the functional variant thereof. The dose administered to the subject may be about 120 mg anifrolumab or the functional variant thereof.

Administration of the dose or unit dose may provide a plasma concentration of anifrolumab or the functional variant thereof in the patient of >10 ug (i.e. 10 μg or more) anifrolumab or the functional variant thereof per ml of plasma (i.e. a plasma concentration of >10 μg/ml). Administration of the dose or unit dose may provide a plasma concentration of anifrolumab or the functional variant thereof in the subject of about 10-100 μg/ml. Administration of the dose or unit dose may provide a plasma concentration of anifrolumab or the functional variant thereof in the subject of about 20-80 ug/ml. Administration of the dose or unit dose may provide a plasma concentration of anifrolumab or the functional variant thereof in the subject of about 30-70 ug/ml. Administration of the dose or unit dose may provide a trough concentration of anifrolumab or the functional variant thereof in the subject of >20 μg/ml (i.e. 20 μg/ml or more). Administration of the dose or unit dose may provide a trough concentration of anifrolumab or the functional variant thereof in the subject of ≥30 μg/ml (i.e. 30 μg/ml or more). Administration of the dose or unit dose may provide a trough concentration of anifrolumab or the functional variant thereof in the subject of ≥40 μg/ml (i.e. 40 μg/ml or more). Administration of the dose or unit dose may provide a trough concentration of anifrolumab or the functional variant thereof in the subject of about 20-100 μg/ml. Administration of the dose or unit dose may provide a trough concentration of anifrolumab or the functional variant thereof in the subject of about 30-80 ug/ml. Administration of the dose or unit dose may provide a trough concentration of anifrolumab or the functional variant thereof in the subject of about 40-70 ug/ml.

The dose or unit dose may provide a therapeutic effect in the subject that is at least equivalent to a therapeutic effect provided by administration of an intravenous dose of 300 mg anifrolumab or the functional variant thereof administered once every (Q4W). The dose or unit dose may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The methods of the invention may comprise administering the dose or unit dose at intervals of 6-8 days. The dose or unit dose may be administered once per week (QW). The dose or unit dose may be 120 mg anifrolumab or the functional variant thereof, wherein the method comprises administering the dose in a single administration step once per week (QW). In other words, the method comprises administering 120 mg QW of anifrolumab of the functional variant thereof. The dose or unit dose may be administered once per week for at least about 4 weeks. The dose or unit dose may be administered once per week for at least about 8 weeks. The dose or unit dose may be administered once per week for at least about 12 weeks. The dose or unit dose may be administered once per week for at least about 16 weeks. The dose or unit dose may be administered once per week for at least about 20 weeks. The dose or unit dose may be administered once per week for at least about 24 weeks. The dose or unit dose may be administered once per week for at least about 28 weeks. The dose or unit dose may be administered once per week for at least about 32 weeks. The dose or unit dose may be administered once per week for about 8 weeks. The dose or unit dose may have a volume permitted it suitable delivery in a single subcutaneous administration step. The dose or unit dose may have a volume of about 0.5 to about 1 ml. The dose or unit dose may have a volume of less than 1 ml. The dose or unit dose may have a volume of about 0.8 ml.

### 4.8. The Subject

The subject may be a human subject. The subject may be an adult. The subject may be a patient with an elevated type I IFN gene signature. The subject may be a type I interferon stimulated gene signature (IFNGS)-test high patient pre-administration with the dose or unit dose. The subject may have elevated of the genes IFI27, IFI44, IFI44L, and RSAD2 in the whole blood. The method may comprise identifying the subject as IFNGS-test high patient pre-treatment with the dose or unit dose. The method may comprise measuring the expression of the genes IFI27, IFI44, IFI44L, and RSAD2 in the whole blood of the subject. The method may comprise measuring the expression of the genes IFI27, IFI44, IFI44L, and RSAD2 in the whole blood of the subject by RT-PCR.

The subject may have moderate to severe SLE pre-treatment with the IFNAR1 inhibitor. Pretreatment with the IFNAR1 inhibitor, the subject may be refractive to treatment with the one more immunomodulators or has relapsed during or following treatment with the one or more immunomodulators. Pretreatment with the IFNAR1 inhibitor the subject may have a SLEDAI-2K score of ≥10 (at least). Pretreatment with the IFNAR1 inhibitor, the subject may have a CLASI activity score of ≥10 (at least 10). Pretreatment with the IFNAR1 inhibitor, the subject may have a swollen and tender joint count of ≥6.

The subject may have moderate to severe SLE as defined by the ACR Classification Criteria for SLE (ACR 1997²⁴ and/or EULAR/ACR 2019²⁵).

### 4.9. Pharmaceutical composition

The invention also relates to a pharmaceutical composition for use in a method of treating CLE in a subject, the method comprising subcutaneously administering the pharmaceutical composition to a subject, wherein the pharmaceutical composition comprises a dose of anifrolumab or functional variant thereof, wherein the dose is >105 mg and <150 mg. The dose of anifrolumab of the functional variant thereof may be a unit dose (unit dose form, pharmaceutical unit dose form, pharmaceutical unit dose). Functional anifrolumab variants include antigen-binding fragments of anifrolumab and antibody and immunoglobulin derivatives of anifrolumab.

In another aspect the invention relates to a pharmaceutical composition for use in a method of treating SLE in a subject, the method comprising subcutaneously administering the pharmaceutical composition to the subject, wherein the pharmaceutical composition comprises a dose of anifrolumab or functional variant thereof, wherein administering the pharmaceutical composition every week provides a plasma concentration in the subject that is at least equivalent to the plasma concentration provided by intravenous administration of 300 mg of anifrolumab or the functional variant thereof every 4 weeks. Administering the dose every week may provide a plasma concentration in the subject that is about equivalent to the plasma concentration provided by intravenous administration of 400 mg of anifrolumab or the functional variant thereof every 4 weeks. The dose may be <150 mg (i.e. less than 150 mg) anifrolumab or the functional variant thereof. The dose may be >105 mg (i.e. more than 105 mg) anifrolumab or the functional variant thereof. The dose may be ≤135 mg (i.e. 135 mg or less) anifrolumab or the functional variant thereof. The dose may be about 120 mg anifrolumab or the functional variant thereof. The dose may be 120 mg anifrolumab or the functional variant thereof.

The pharmaceutical composition may be administered at intervals of 6-8 days. The pharmaceutical composition may be administered once per week (QW). The pharmaceutical composition may be administered in a single administration step. The dose may be 120 mg anifrolumab or the functional variant thereof, and the method of treatment may comprise administering the dose in a single administration step once per week (QW). The pharmaceutical composition may be administered once per week for at least about 4 weeks. The pharmaceutical composition may be administered once per week for at least about 8 weeks. The dose or unit dose may be administered once per week for at least about 12 weeks. The pharmaceutical composition may be administered once per week for at least about 16 weeks. The pharmaceutical composition may be administered once per week for at least about 20 weeks. The pharmaceutical composition may be administered once per week for at least about 24 weeks. The pharmaceutical composition may be administered once per week for at least about 28 weeks. The pharmaceutical composition may be administered once per week for at least about 32 weeks. The pharmaceutical composition may be administered once per week for about 8 weeks. The pharmaceutical composition may have a volume permitted it suitable delivery in a single subcutaneous administration step. The pharmaceutical composition may have a volume of about 0.5 to about 1 ml. The pharmaceutical composition may have a volume of less than 1 ml. The pharmaceutical composition may have a volume of about 0.8 ml.

Administration of the pharmaceutical composition may provide a plasma concentration of anifrolumab or the functional variant thereof in the patient of >10 ug (i.e. 10 μg or more) anifrolumab or the functional variant thereof per ml of plasma (i.e. a plasma concentration of >10 μg/ml). Administration of the pharmaceutical composition may provide a plasma concentration of anifrolumab or the functional variant thereof in the subject of about 10-100 μg/ml. Administration of the pharmaceutical composition may provide a plasma concentration of anifrolumab or the functional variant thereof in the subject of about 20-80 ug/ml. Administration of the pharmaceutical composition may provide a plasma concentration of anifrolumab or the functional variant thereof in the subject of about 30-70 ug/ml. Administration of the pharmaceutical composition may provide a trough concentration of anifrolumab or the functional variant thereof in the subject of ≥20 μg/ml (i.e. 20 μg/ml or more). Administration of the pharmaceutical composition may provide a trough concentration of anifrolumab or the functional variant thereof in the subject of ≥30 μg/ml (i.e. 30 μg/ml or more). Administration of the pharmaceutical composition may provide a trough concentration of anifrolumab or the functional variant thereof in the subject of ≥40 μg/ml (i.e. 40 μg/ml or more). Administration of the pharmaceutical composition may provide a trough concentration of anifrolumab or the functional variant thereof in the subject of about 20-100 μg/ml. Administration of the pharmaceutical composition may provide a trough concentration of anifrolumab or the functional variant thereof in the subject of about 30-80 ug/ml. Administration of the pharmaceutical composition may provide a trough concentration of anifrolumab or the functional variant thereof in the subject of about 40-70 g/ml.

The pharmaceutical composition may provide a therapeutic effect in the subject that is at least equivalent to a therapeutic effect provided by administration of an intravenous dose of 300 mg anifrolumab or the functional variant thereof administered once every (Q4W). The pharmaceutical composition may provide a trough concentration of anifrolumab or the functional variant thereof in the subject that is greater than a trough concentration of anifrolumab or the functional variant thereof provided by administration of an intravenous dose of 300 mg anifrolumab or the functional variant thereof once every 4 weeks (Q4W). The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

### 4.10. Device

The invention also relates to an injection device comprising the unit dose of the invention, or the pharmaceutical composition for the use of any of the invention.

The pharmaceutical in the injection device may comprise >105 mg (i.e. more than 105 mg) and <150 mg (i.e. less than 150 mg) anifrolumab or a functional variant thereof. The pharmaceutical composition in the injection device may comprise about 120 mg anifrolumab or the functional variant thereof. The pharmaceutical composition in the injection device may comprise 120 mg anifrolumab or the functional variant thereof. The concentration of anifrolumab or the functional variant thereof in the pharmaceutical composition in the injection device may be 150 mg/ml. The volume of the pharmaceutical composition in the injection device may be at least about 0.8 ml. The volume of the pharmaceutical composition may be about 0.8 ml.

The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition in the injection device may comprise 50 mM lysine HCl. The pharmaceutical composition may comprise 130 mM trehalose dihydrate. The pharmaceutical composition in the injection device may comprise about 150 to 200 mg/ml anifrolumab or the functional variant thereof, about 25 to 150 mM of lysine sale and an uncharged excipient. The pharmaceutical composition in the injection device may comprise 150 mg/mL anifrolumab or the functional variant thereof. The pharmaceutical composition may comprise 50 mM lysine HCl. The pharmaceutical composition in the injection device may comprise 130 mM trehalose dihydrate. The pharmaceutical composition in the injection device may comprise 0.05% polysorbate 80. The pharmaceutical composition in the injection device may comprise 25 mM histidine/histidine HCl. The pharmaceutical composition in the injection device may comprise 150 mg/mL anifrolumab or the functional variant thereof, 50 mM lysine HCl, 130 mM trehalose dihydrate, 0.05% polysorbate 80 and 25 mM histidine/histidine HCl.

In another aspect the invention relates to an injection device comprising a unit dose. The unit dose may comprise >105 mg (i.e. at least 105 mg) and <150 mg (i.e. less than 150 mg) anifrolumab or a functional variant thereof. The unit dose may comprise ≤135 mg (i.e. 135 mg or less) anifrolumab or the functional variant thereof. The unit dose may comprise about 120 mg anifrolumab or the functional variant thereof. The unit dose in the injection device may comprise 120 mg anifrolumab or the functional variant thereof. The unit dose in the injection device may consist essentially of >105 mg and <150 mg anifrolumab or the functional variant thereof. The unit dose in the injection device may consist essentially of ≤135 mg anifrolumab or the functional variant thereof. The unit dose in the injection device may consist essentially of about 120 mg anifrolumab or the or the functional variant thereof. The concentration of anifrolumab or the functional variant thereof in the unit dose in the injection device may be about 150 mg/ml. The volume of the unit dose in the injection device may be less than 1 ml. The unit dose in the injection device may have a volume of about 0.5 to about 1 ml. The concentration of the unit dose may be about 0.8 ml. The volume of the unit dose may be 0.8 ml. The unit dose in the injection device may comprise a formulation of about 150 to 200 mg/ml anifrolumab or the functional variant thereof, about 25 to 150 mM of lysine sale and an uncharged excipient. The unit dose in the injection device may comprise a formulation of 150 to 200 mg/ml anifrolumab or the functional variant thereof, 25 to 150 mM of lysine sale and an uncharged excipient. The unit dose comprises a formulation of 25 mM histidine-HCL, 130 mM trehalose, and 0.05% w/v polysorbate 80. The formulation may have a pH of about 5.9.

The injection device may be a pre-filled syringe (PFS). The injection device may be an accessorized pre-filed syringe (AFPS). The injection device may be an auto-injector (Al).

### 4.11. Kit

In another aspect the invention relates to a kit comprising a unit dose of the invention and instructions for use, wherein the instructions for use comprise instructions for subcutaneous administration of the unit dose to a subject.

In another aspect the invention relates to a kit comprising the pharmaceutical composition for the use of the invention, wherein the instructions for use comprise instructions for subcutaneous administration of the pharmaceutical composition to a subject.

In another aspect the invention relates to a kit comprising the injection device of any of the invention, and instructions for use, wherein the instruction for use comprise instructions for use of the injection device to subcutaneously administer the unit dose or pharmaceutical composition to the subject.

The kit of the invention may comprise packaging, wherein the packaging is adapted to hold the injection device and the instructions for use. The instructions for use may be attached to the injection device. The instruction for use may comprise instructions for administration of >105 mg and <150 mg anifrolumab or functional variant thereof. The instruction for use may comprise instructions for administration of ≤135 mg anifrolumab or the functional variant thereof. The instruction for use may comprise instructions for administration of 120 mg anifrolumab or the functional variant thereof. The instruction for use may comprise instructions for administration of 120 mg anifrolumab or the functional variant thereof every 4 weeks. The instructions for use may define the subject as having a type I IFN mediated disease. The instructions may define the subject as having SLE. The instructions may define the subject as having moderate to severe SLE. The instructions for use may be written instructions.

The instructions for use may specify that the injection device, unit dose and/or pharmaceutical composition are for use in the treatment of SLE. The instructions for use comprise instructions for administration of 120 mg anifrolumab or the functional variant thereof every week. The instructions for use may specify that the unit dose or pharmaceutical composition of the invention are for use in the treatment of a subject that is refractory or unresponsive to treatment with the one or more immunomodulators. The instructions for use may specify that the unit dose or pharmaceutical composition of the invention is for use in the treatment of a subject that is refractory or unresponsive to treatment with the one or more immunomodulators. The instruction for use may specify that the unit dose or pharmaceutical composition of the invention is for use in any of the method of the invention. The instructions for use may specify that the method of the invention has been demonstrated in a phase III clinical trial.

### 4.12. Formulations

The anifrolumab or the functional variant thereof may be comprised within a pharmaceutical composition. The pharmaceutical composition may comprise about 150 to 200 mg/ml anifrolumab or the functional variant thereof, about 25 to 150 mM of lysine sale and an uncharged excipient. The pharmaceutical composition may comprise 150 mg/mL anifrolumab or the functional variant thereof. The pharmaceutical composition may comprise 50 mM lysine HCl. The pharmaceutical composition may comprise 130 mM trehalose dihydrate. The pharmaceutical composition may comprise 0.05% polysorbate 80. The pharmaceutical composition may comprise 25 mM histidine/histidine HCl. The pharmaceutical composition may comprise 150 mg/mL anifrolumab or the functional variant thereof, 50 mM lysine HCl, 130 mM trehalose dihydrate, 0.05% polysorbate 80 and 25 mM histidine/histidine HCl.

Stable formulations suitable for administration to subjects and comprising anifrolumab are described in detail in U.S. Pat. No. 10,125,195 B1, which is incorporated herein in its in entirety.

### 5. DEFINITIONS

### 5.1. IFNAR Inhibitors

Anifrolumab (MEDI-546, anifro, ANI) is a human immunoglobulin G1 kappa (IgG1κ) monoclonal antibody (mAb) directed against subunit 1 of the type I interferon receptor (IFNAR1). Anifrolumab downregulates IFNAR signaling and suppresses expression of IFN-inducible genes. Disclosures related to anifrolumab can be found in U.S. Pat. Nos. 7,662,381 and 9,988,459, which are incorporated herein by reference in their entirety. Sequence information for anifrolumab is provided in Table 5-1: Anifrolumab Sequences.

TABLE 5-1
Anifrolumab Sequences

| | |
|---|---|
| Anifrolumab VH (SEQ ID NO: 1) | EVQLVQSGAEVKKPGESLKISCKGSGYIFTNYWIAWVRQMPGKGLESMG |
| | IIYPGDSDIRYSPSFQGQVTISADKSITTAYLQWSSLKASDTAMYYCARHDI |
| | EGFDYWGRGTLVTVSS |
| Anifrolumab VL (SEQ ID NO: 2) | EIVLTQSPGTLSLSPGERATLSCRASQSVSSSFFAWYQQKPGQAPRLLIY |
| | GASSRATGIPDRLSGSGSGTDFTLTITRLEPEDFAVYYCQQYDSSAITFG |
| | QGTRLEIK |
| HCDR1 (SEQ ID NO: 3) | NYWIA |
| HCDR2 (SEQ ID NO: 4) | IIYPGDSDIRYSPSFQG |
| HCDR3 (SEQ ID NO: 5) | HDIEGFDY |
| LCDR1 (SEQ ID NO: 6) | RASQSVSSSFFA |
| LCDR2 (SEQ ID NO: 7) | GASSRAT |
| LCDR3 (SEQ ID NO: 8) | QQYDSSAIT |
| Light chain constant region | RTVAAPSVFIFPPSDEQLKSGTASVVCLLNNFYPREAKVQWKVDNALQS |
| (SEQ ID NO: 9) | GNSQESVTEQDSKDSTYSLSSTLTLSKADYEKHKVYACEVTHQGLSSPV |
| | TKSFNRGEC |
| Heavy chain constant region | ASTKGPSVFPLAPSSKSTSGGTAALGCLVKDYFPEPVTVSWNSGALTSG |
| (SEQ ID NO: 10) | VHTFPAVLQSSGLYSLSSVVTVPSSSLGTQTYICNVNHKPSNTKVDKRVE |
| | PKSCDKTHTCPPCPAPEFEGGPSVFLFPPKPKDTLMISRTPEVTCVVVDV |
| | SHEDPEVKFNWYVDGVEVHNAKTKPREEQYNSTYRVVSVLTVLHQDWL |
| | NGKEYKCKVSNKALPASIEKTISKAKGQPREPQVYTLPPSREEMTKNQVS |
| | LTCLVKGFYPSDIAVEWESNGQPENNYKTTPPVLDSDGSFFLYSKLTVDK |
| | SRWQQGNVFSCSVMHEALHNHYTQKSLSLSPGK |
| Heavy chain | EVQLVQSGAEVKKPGESLKISCKGSGYIFTNYWIAWVRQMPGKGLESMG |
| (SEQ ID NO: 11) | IIYPGDSDIRYSPSFQGQVTISADKSITTAYLQWSSLKASD TAMYYCARHD |
| | IEGFDYWGRGTLVTVSSASTKGPSVFPLAPSSKSTSGGTAALGCLVKDYF |
| | PEPVTVSWNSGALTSGVHTFPAVLQSSGLYSLSSVVTVPS SSLGTQTYIC |
| | NVNHKPSNTKVDKRVEPKSCDKTHTCPPCPAPEFEGGPSVFLFPPKPKD |
| | TLMISRTPEVTCVVVDVSHEDPEVKFNWYVDGVEVHNAKTK |
| | PREEQYNSTYRVVSVLTVLHQDWLNGKEYK CKVSNKALPA SIEKTISKAK |
| | GQPREPQVYTLPPSREEMTKNQVSLTCLVKGFYPSDIAVEWESNGQPEN |
| | NYKTTPPVLDSDGSFFLYSKLTVDKSRWQQGNVFSCSVMHEALHNHYT |
| | QKS LSLSPGK |
| Light chain | EIVLTQSPGTLSLSPGERATLSCRASQSVS SSFFAWYQQK PGQAPRLLIY |
| (SEQ ID NO: 12) | GASSRATGIPDRLSGSGSGT DFTLTITRLE PEDFAVYYCQ QYDSSAITFG |
| | QGTRLEIKRTVAAPSVFIFPPSDEQLKSGT ASVVCLLNNF YPREAKVQWK |
| | VDNALQSGNSQESVTEQDSKDSTYSLSSTLTLSKADYEKHKVYACEVTH |
| | QGLSSPVTKSFNRGEC |

Anifrolumab is an immunoglobulin comprising an HCDR1, HCDR2 and HCDR3 of SEQ ID NO: 3, SEQ ID NO: 4, and SEQ ID NO: 5, respectively (or functional variant thereof); and an LCDR1, LCDR2 and LCDR3 of SEQ ID NO: 6, SEQ ID NO: 7, and SEQ ID NO: 8, respectively (or functional variant thereof). Anifrolumab is an immunoglobulin comprising a VH of SEQ ID NO: 1 and a VL of SEQ ID NO: 2.

The constant region of anifrolumab has been modified such that anifrolumab exhibits reduced affinity for at least one Fc ligand compared to an unmodified antibody. Anifrolumab is a modified IgG class monoclonal antibody specific for IFNAR1 comprising in the Fc region an amino acid substitution of L234F, as numbered by the EU index as set forth in Kabat (1991, NIH Publication 91-3242, National Technical Information Service, Springfield, Va.). Anifrolumab is a modified IgG class monoclonal antibody specific for IFNAR1 comprising in the Fc region an amino acid substitution of L234F, L235E and/or P331S, as numbered by the EU index as set forth in Kabat (1991, NIH Publication 91-3242, National Technical Information Service, Springfield, Va.). Anifrolumab is an antibody comprising a light chain constant region of SEQ ID NO: 9. Anifrolumab is an antibody comprising a heavy chain constant region of SEQ ID NO: 10. Anifrolumab is an antibody comprising a light chain constant region of SEQ ID NO: 9 and a heavy chain constant region of SEQ ID NO: 10. Anifrolumab is an antibody comprising a heavy chain of SEQ ID NO: 11. Anifrolumab is an antibody comprising a light chain of SEQ ID NO: 12. Anifrolumab is an antibody comprising a heavy chain of SEQ ID NO: 11 and a light chain of SEQ ID NO: 12.

Functional variants of anifrolumab are sequence variants that perform the same function as anifrolumab. Functional variants of anifrolumab are variants that bind the same target as anifrolumab and have the same effector function as anifrolumab. Functional anifrolumab variants include antigen-binding fragments of anifrolumab and antibody and immunoglobulin derivatives of anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

For example, a variant of the reference (anifrolumab) antibody may comprise: a heavy chain CDR1 having at most 2 amino acid differences when compared to SEQ ID NO: 3; a heavy chain CDR2 having at most 2 amino acid differences when compared to SEQ ID NO: 4; a heavy chain CDR3 having at most 2 amino acid differences when compared to SEQ ID NO: 5; a light chain CDR1 having at most 2 amino acid differences when compared to SEQ ID NO: 6; a light chain CDR2 having at most 2 amino acid differences when compared to SEQ ID NO: 7; and a light chain CDR3 having at most 2 amino acid differences when compared to SEQ ID NO: 8; wherein the variant antibody binds to the target of anifrolumab (e.g. IFNAR) and preferably with the same affinity.

A variant of the reference (anifrolumab) antibody may comprise: a heavy chain CDR1 having at most 1 amino acid difference when compared to SEQ ID NO: 3; a heavy chain CDR2 having at most 1 amino acid difference when compared to SEQ ID NO: 4; a heavy chain CDR3 having at most 1 amino acid difference when compared to SEQ ID NO: 5; a light chain CDR1 having at most 1 amino acid differences when compared to SEQ ID NO: 6; a light chain CDR2 having at most 1 amino acid difference when compared to SEQ ID NO: 7; and a light chain CDR3 having at most 1 amino acid difference when compared to SEQ ID NO: 8; wherein the variant antibody binds to the target of anifrolumab (e.g. IFNAR) optionally with the same affinity.

A variant antibody may have at most 5, 4 or 3 amino acid differences total in the CDRs thereof when compared to a corresponding reference (anifrolumab) antibody, with the proviso that there is at most 2 (optionally at most 1) amino acid differences per CDR. A variant antibody may have at most 2 (optionally at most 1) amino acid differences total in the CDRs thereof when compared to a corresponding reference (anifrolumab) antibody, with the proviso that there is at most 2 amino acid differences per CDR. A variant antibody may have at most 2 (optionally at most 1) amino acid differences total in the CDRs thereof when compared to a corresponding reference (anifrolumab) antibody, with the proviso that there is at most 1 amino acid difference per CDR.

A variant antibody may have at most 5, 4 or 3 amino acid differences total in the framework regions thereof when compared to a corresponding reference (anifrolumab) antibody, with the proviso that there is at most 2 (optionally at most 1) amino acid differences per framework region. Optionally a variant antibody has at most 2 (optionally at most 1) amino acid differences total in the framework regions thereof when compared to a corresponding reference (anifrolumab) antibody, with the proviso that there is at most 2 amino acid differences per framework region. Optionally a variant antibody has at most 2 (optionally at most 1) amino acid differences total in the framework regions thereof when compared to a corresponding reference (anifrolumab) antibody, with the proviso that there is at most 1 amino acid difference per framework region.

A variant antibody may comprise a variable heavy chain and a variable light chain as described herein, wherein: the heavy chain has at most 14 amino acid differences (at most 2 amino acid differences in each CDR and at most 2 amino acid differences in each framework region) when compared to a heavy chain sequence herein; and the light chain has at most 14 amino acid differences (at most 2 amino acid differences in each CDR and at most 2 amino acid differences in each framework region) when compared to a light chain sequence herein; wherein the variant antibody binds to the same target antigen as the reference (anifrolumab) antibody (e.g. IFNAR) and preferably with the same affinity.

The variant heavy or light chains may be referred to as “functional equivalents” of the reference heavy or light chains. A variant antibody may comprise a variable heavy chain and a variable light chain as described herein, wherein: the heavy chain has at most 7 amino acid differences (at most 1 amino acid difference in each CDR and at most 1 amino acid difference in each framework region) when compared to a heavy chain sequence herein; and the light chain has at most 7 amino acid differences (at most 1 amino acid difference in each CDR and at most 1 amino acid difference in each framework region) when compared to a light chain sequence herein; wherein the variant antibody binds to the same target antigen as the reference (anifrolumab) antibody (e.g. IFNAR) and optionally with the same affinity.

Functional variants of anifrolumab include the antibodies described in WO 2018/023976 A1, incorporated herein by reference (Table 5-2).

TABLE 5-2
anti-IFNAR antibody sequences

| | | | | |
|---|---|---|---|---|
| H15D10 | 19 | VH | EVQLVQSGAE VKKPGESLRISC | SEQ ID |
| | | | KGSGYTFT NYWVAWVRQM | NO: 13 |
| | | | PGKGLESMGI IYPGDSDTRY | |
| | | | SPSFQGHVTI SADKSISTAY | |
| L8C3 | 21 | VL | DIQMTQSPSSLSASLGDRVTITCRAS | SEQ ID |
| | | | QNVGNYLNWYQQKPGKAPKLLIYRA | NO: 14 |
| | | | SNLASGVPSRFSGSGSGTDFTLTISS | |
| | | | LQPEDFATYYCQQMEHAPPTFGQG | |
| | | | TKVEIKR | |
| L16C11 | 22 | VL | EIVLTQSPGTLSLSPGERATLSCRAS | SEQ ID |
| | | | QSVIGYYLAWYQQKPGQAPRLLIYS | NO: 15 |
| | | | VSTLASGIPDRFSGSGSGTDFTLTIS | |
| | | | RLEPEDFAVYYCQQYYRFPITFGQG | |
| | | | TKVEIK | |
| H19B7 | 20 | VH | EVQLVQSGAE VKKPGESLRI | SEQ ID |
| | | | SCKGSGYTFT NYWMAWVRQM | NO: 16 |
| | | | PGKGLESMGI IYPSDSDTRY | |
| | | | SPSFQGHVTI SADKSISTAY | |
| | | | LQWSSLKASD TAMYYCARHD | |
| | | | VEGYDYWGQG TLVTVSS | |

Functional variants include antibodies comprising the VH amino acid sequence SEQ ID NO: 13. Functional variants include antibodies comprising the VH amino acid sequence SEQ ID NO: 16. Functional variants include antibodies comprising the VL amino acid sequence SEQ ID NO: 14. Functional variants include antibodies comprising the VL amino acid sequence SEQ ID NO: 15. Functional variants include antibodies comprising the VL amino acid sequence SEQ ID NO: 16. Functional variants include antibodies comprising the VH sequence SEQ ID NO: 13 and VL amino acid sequence SEQ ID NO: 16. Functional variants include antibodies comprising the VH sequence SEQ ID NO: 13 and VL amino acid sequence SEQ ID NO: 15. Functional variants include antibodies comprising the VH sequence SEQ ID NO: 16 and VL amino acid sequence SEQ ID NO: 15. Functional variants include antibodies comprising the VH sequence SEQ ID NO: 16 and VL amino acid sequence SEQ ID NO: 14.

IFNAR inhibitors may be a monoclonal antibody comprising the VH amino acid sequence SEQ ID NO: 13. The anti-IFNAR antibodies may comprise the VH amino acid sequence SEQ ID NO: 16. The anti-IFNAR antibodies may comprise the VL amino acid sequence SEQ ID NO: 14. The anti-IFNAR antibodies may comprise the VL amino acid sequence SEQ ID NO: 15. The anti-IFNAR antibodies may comprise the VL amino acid sequence SEQ ID NO: 16. The anti-IFNAR antibodies may comprise the VH sequence SEQ ID NO: 13 and VL amino acid sequence SEQ ID NO: 16. The anti-IFNAR antibodies may comprise the VH sequence SEQ ID NO: 13 and VL amino acid sequence SEQ ID NO: 15. The anti-IFNAR antibodies may comprise the VH sequence SEQ ID NO: 16 and VL amino acid sequence SEQ ID NO: 15. The anti-IFNAR antibodies may comprise the VH sequence SEQ ID NO: 16 and VL amino acid sequence SEQ ID NO: 14.

### 5.1.1. Anifrolumab in the Clinic

Anifrolumab safety has been evaluated in 8 blinded or open-label intravenous (IV) and subcutaneous (SC) studies: 6 studies in patients with SLE (Study 05, Study 04, Study 1013, Study 1145, and Study 08), 1 study in patients with systemic sclerosis (SSc) (Study MI-CP180), and 1 study in healthy volunteers (Study 06)

Table 5-3). Of these studies, two (Studies 08 and 06) employed SC anifrolumab administration. Two studies are ongoing: 1 study in patients with SLE (Study 09) and 1 study in patients with lupus nephritis (LN) (Study 07).

TABLE 5-3
Anifrolumab clinical studies

| | | Subjects | Admin. | Anifro Dose | CT.gov |
|---|---|---|---|---|---|
| Phase III studies | | | | | |
| Study 05 | TULIP II | SLE patients | IV | 300 mg Q4W | NCT02446899 |
| Study 04 | TULIP I | SLE patients | IV | 300 mg Q4W | NCT02962960 |
| Study 09 | Long-term | SLE patients | IV | 300 mg | |
| | extension | | | | |
| Phase II studies | | | | | |
| Study 1013 | MUSE | SLE patients | IV | 300 mg or | NCT01438489 |
| | | | | 1000 mg Q4W | |
| Study 1145 | MUSE OLE | | IV | 300 mg | NCT01753193 |
| Study 08 | | SLE patients | SC | | NCT02962960 |
| Study 07 | | LN patients | | | NCT02547922 |
| Phase I | | | | | |
| Study MI-CP180 | | Scleroderma patients | IV | | |
| Study 0626 | | Healthy volunteers | IV and SC | 300 mg, SC, | NCT02601625 |
| | | | | 300 mg IV or | |
| | | | | 600 mg SC | |

Study 1013 is described in further detail in Furie et al. 201715, which is incorporated herein by reference in its entirety. Study 04 is described in further detail in Furie et al. 201916, which is incorporated herein by reference in its entirety. The results of Study 05 are presented in Morand et al. 202014, herein incorporated by reference in its entirety. A full summary of the evidence for intravenous anifrolumab clinical efficacy in SLE is provided in Tanaka et al., 202027, which is incorporated herein by reference in its entirety.

### 5.2. Immunomodulators

### 5.2.1. Abatacept

Abatacept (Orencia) is a CTLA-4 fusion protein (CTLA4-Ig) that binds to CD80/86 on the surface of antigen presenting cells and blocks signaling through CD-28 required for T-cell activation. In preclinical studies abatacept was demonstrated to have immunomodulatory activity in the NZB/NZW murine model of lupus²⁸. Abatacept for treatment of non-renal SLE was been evaluated in a phase IIb, randomized, double-blind, placebo-controlled trial²⁹ (NCT00119678). The primary end point was the proportion of patients with new flare (adjudicated) according to a score of A/B on the British Isles Lupus Assessment Group (BILAG) index after the start of the steroid taper. The primary and secondary end points were not met.

TABLE 5-4
Abatacept dosage and administration

| Study | Route | Dose | Indication |
|---|---|---|---|
| NCT00119678 | Intravenous | 10 mg/kg every 28 days | SLE |

### 5.2.2. Atacicept

Atacicept (TACI-Ig) is a fully human recombinant fusion protein that neutralizes both BAFF and APRIL. Atacicept (SEQ ID NO: 13) is described in U.S. Pat. No. 5,851,795, which is incorporated herein by reference. Atacicept is in clinical development for treatment of Rheumatoid Arthritis, Juvenile Idiopathic Arthritis and Psoriatic Arthritis. In a Phase 2/3 clinical trial (NCT00624338), atacicept was administered to SLE patients at a subcutaneous dose of 75 mg or 150 mg.

A phase Ib study investigated the safety and tolerability of atacicept given intravenously at different doses ranging from 3 mg/kg once to 2×9 mg/kg over three weeks in comparison to placebo. Atacicept administered intravenously was found to be generally well tolerated, both systemically and locally, in patients with mild-to-moderate SLE30.

In the ADDRESS II trial (NCT01972568), a phase IIb placebo-controlled study, atacicept was given weekly at a dose of either 75 or 150 mg. The proportion of patients with a SRI (4) at 24 weeks was assessed as primary endpoint. More patients in the 75 mg atacicept, but not in the 150 mg atacicept group, reached the primary endpoint (p=0.045). AEs were reported slightly more frequently in patients receiving atacicept, whereas a higher percentage of SAEs were noted in the placebo group³¹. Dosage information for atacicept is summarized in Table 5-5.

TABLE 5-5
Atacicept dosage and administration

| Study | Route | Dose | | Indication |
|---|---|---|---|---|
| Pena-Rossi | Intravenous | single dose: 3, 9 | | SLE |
| et al³⁰ | | or 18 mg/kg; or multiple | | |
| | | dose: 2 × 9 mg/kg | | |
| NCT00624338 | Subcutaneous | 75 mg twice weekly for | | SLE |
| | | 4 weeks, then QW | | |
| NCT00624338 | Subcutaneous | 150 mg twice weekly for | | SLE |
| | | 4 weeks, then QW | | |
| NCT01972568 | Subcutaneous | 75 | mg QW | SLE |
| NCT01972568 | Subcutaneous | 150 | mg QW | SLE |

### 5.2.3. Belimumab (Benlysta)

Belimumab (Benlysta) is an anti-BAFF (BLyS) antibody. Belimumab is described in U.S. Pat. No. 7,138,501, which is incorporated herein by reference. Dosage information for belimumab is provided in Table 5-6. Belimumab is approved for the treatment of SLE administered by intravenous infusion, at a dose of 10 mg/kg at 2-week intervals for the first 3 doses and at 4-week intervals thereafter. Belimumab is also approved for the treatment of SLE administered by subcutaneous injection, at a dose of 200 mg once weekly. Belimumab formulations are described in US patent application US20180289804 A1 which is incorporated herein by reference in its entirety.

TABLE 5-6
Belimumab dosage and administration

| Route | Dose | Use/Indication |
|---|---|---|
| Intravenous | 10 mg/kg Q2W for three weeks, | FDA approved |
| | then 10 mg/kg Q4W | for SLE |
| Subcutaneous | 200 mg QW | FDA approved |
| | | for SLE |

### 5.2.4. Epratuzumab

Epratuzumab is a monoclonal antibody that modulates B-cell activity by binding CD22 on the surface of mature B-cells. Epratuzumab is described in U.S. Pat. Nos. 5,789,554 and 6,187,287, which are incorporated herein by reference. Epratuzumab is in development for the treatment of leukemia, acute lymphocytic leukemia, Non-Hodgkin's Lymphoma (Diffuse Large B cell lymphoma, and Follicular lymphoma).

ALLEVIATE-1 and ALLEVIATE-2 were 2 phase IIb multicentre, multinational, double blinded, placebo-controlled, randomised controlled trials, looking at the efficacy and safety of epratuzumab in SLE³². The trials were discontinued early due to drug shortage and the data were combined for analysis. ALLEVIATE-1 and ALLEVIATE-2 studied patients with at least 1 BILAG A organ system and at least 2 BILAG B organ systems at baseline respectively. In ALLEVIATE-1 patients were randomised to individualized standard of care (SOC) plus either epratuzumab 360 mg/m² or epratuzumab 720 mg/m² or placebo. The study failed to meet its primary outcome with there being no significant difference in BILAG response at week 12.

In multicentre phase III trials EMBODY 1 (NCT01262365) and EMBODY 2 (NCT01261793) for Epratuzumab in patients with moderate-to-severe SLE, the primary efficacy endpoint, BICLA response at 48 weeks, was not met³³. EMBODY 1 and EMBODY 2 epratuzumab was administered at a dose of 600 mg infusions delivered weekly for a total of 4 weeks (cumulative dose 2400 mg) over four 12-week treatment cycles or at a dose of 1200 mg infusions delivered every other week for a total of 4 weeks (cumulative dose 2400 mg) over four 12-week treatment cycles³⁴.

TABLE 5-7
Epratuzumab dosage and administration

| Study | Route | Dose | Use/Indication |
|---|---|---|---|
| Dorner et al.³⁵ | Intravenous | 360 mg/m² Q2W | Investigative, SLE |
| ALLEVIATE I (SL0003) and | Intravenous | 360 mg/m² Q2W or | Investigative, SLE |
| ALLEVIATE II (SL0004) | | 720 mg/m² Q2W | |
| EMBODY 1 (NCT01262365) and | Intravenous | 600 mg QW | Investigative, SLE |
| EMBODY 2 (NCT01261793) | | | |

### 5.2.5. Rituximab (Rituxan)

Rituximab (Rituxan) is a chimeric anti-CD20 monoclonal antibody. Rituximab is an effective treatment in a number of autoimmune diseases, including rheumatoid arthritis and ANCA vasculitis. Rituximab is described in U.S. Pat. No. 7,422,739, which is incorporated herein by reference.

Rituximab is approved for the treatment of adult patients with Non-Hodgkin's Lymphoma (NHL), Chronic Lymphocytic Leukemia (CLL), Rheumatoid Arthritis (RA) in combination with methotrexate in adult patients with moderately-to severely-active RA, Granulomatosis with Polyangiitis (GPA) (Wegener's Granulomatosis) and Microscopic Polyangiitis (MPA) in adult and pediatric patients 2 years of age and older in combination with glucocorticoids and Moderate to severe Pemphigus Vulgaris (PV) in adult patients.

The efficacy and safety of rituximab in patients with moderate to severe SLE was evaluated in a multicentre placebo randomized controlled phase II/III trial (EXPLORER) 36. The study randomized patients with baseline active SLE (defined as ≥1 new BILAG A scores or >2 BILAG B scores) to rituximab or placebo. Patients were randomized at a ratio of 2:1 to receive rituximab (1,000 mg) or placebo on days 1, 15, 168, and 182. The primary endpoint was the proportion of rituximab versus placebo-treated patients achieving a complete clinical response (CCR), partial clinical response (PCR), or no response at week 52. The primary endpoint was not met, with similar rates of complete and partial responses in rituximab and placebo arms at 52 weeks. Differences in time to first moderate or severe flare and change in HRQOL were also not significant³⁶. Rituximab is also used off-label in patients with SLE, at a dose of 375 mg/m² for four doses, or 1000 mg/m² for 2 doses³⁷. Rituximab is also being investigated as a treatment of SLE in combination with belimumab (NCT03312907).

Rituximab is administered intravenously. Dose information is provided in Table 5-8.

TABLE 5-8
Rituximab dosage and administration

| Study | Route | Dose | Use/Indication |
|---|---|---|---|
| | Intravenous | 375 mg/m² | FDA approved for NHL |
| | Intravenous | 375 mg/m² in the first cycle and 500 mg/m² in | FDA approved for CLL |
| | | cycles 2-6, in combination with FC, | |
| | | administered every 28 days | |
| | Intravenous | two-1,000 mg intravenous infusions separated | FDA approved for RA |
| | | by 2 weeks (one course) every 24 weeks | |
| | Intravenous | 375 mg/m² once weekly for 4 weeks; The follow | FDA approved for GPA |
| | | up dose for adult patients with GPA and MPA | and MPA |
| | | who have achieved disease control with | |
| | | induction treatment, in combination with | |
| | | glucocorticoids is two 500 mg intravenous | |
| | | infusions separated by two weeks, followed by a | |
| | | 500 mg intravenous infusion every 6 months | |
| | | thereafter based on clinical evaluation | |
| | Intravenous | The dose for PV is two-1,000 mg intravenous | FDA approved for PV |
| | | infusions separated by 2 weeks in combination | |
| | | with a tapering course of glucocorticoids, then a | |
| | | 500 mg intravenous infusion at Month 12 and | |
| | | every 6 months thereafter or based on clinical | |
| | | evaluation. Dose upon relapse is a 1,000 mg | |
| | | intravenous infusion with considerations to | |
| | | resume or increase the glucocorticoid dose | |
| | | based on clinical evaluation. Subsequent | |
| | | infusions may be no sooner than 16 weeks after | |
| | | the previous infusion Methylprednisolone 100 | |
| | | mg intravenous or equivalent glucocorticoid is | |
| | | recommended 30 minutes prior to each infusion. | |
| EXPLORER | Intravenous | 1,000 mg on days 1, 15, 168, and 182 | SLE |
| (NCT00137969)³⁶ | | | |
| | Intravenous | 375 mg/m2 × 4 or 1000 mg × 2 | Open-label use for SLE |

### 5.2.6. Tabalumab

Tabalumab (LY2127399) is a human IgG4 monoclonal antibody that binds both soluble and membrane-bound B-cell activating factor (BAFF). The efficacy and safety of tabalumab was assessed in two 52-week, phase III, multicentre randomized, double-blind, placebo-controlled trial in patients with moderate-to-severe SLE (ILLUMINATE-1 and ILLUMINATE-2). The primary endpoint was proportion of patients achieving SLE Responder Index 5 (SRI-5) response at week 52. In ILLUMINATE-1 (NCT01196091), the primary endpoint was not met. Key secondary efficacy endpoints (OCS sparing, time to severe flare, worst fatigue in the last 24 hours) also did not achieve statistical significance, despite pharmacodynamic evidence of tabalumab biological activity (significant decreases in anti-dsDNA, total B-cells, and immunoglobulins)³⁸. The primary endpoint was met in ILLUMINATE-2 (NCT01205438) in the higher dose group (tabalumab 120 mg every 2 weeks). However, no secondary endpoints were met, including OCS sparing³⁹. Following ILLUMINATE-1 and ILLUMINATE-2, tabalumab development was suspended given the small effect size and inability to meet other important clinical endpoints. Dose information is provided in Table 5-9.

TABLE 5-9
Tabalumab dosage and administration

| | | | Use/ |
|---|---|---|---|
| Study | Route | Dose | Indication |
| ILLUMINATE-1 | subcutaneous | loading dose (240 mg) at | SLE |
| | | week 0 and followed by | |
| | | 120 mg every two weeks, | |
| | | 120 mg every four weeks | |
| ILLUMINATE-1 | subcutaneous | 120 mg every four weeks | SLE |
| ILLUMINATE-2 | subcutaneous | loading dose (240 mg) at | SLE |
| | | week 0 and followed by | |
| | | 120 mg every two weeks, | |
| | | 120 mg every four weeks | |
| ILLUMINATE-2 | subcutaneous | 120 mg every four weeks | SLE |

### 5.2.7. Sifalimumab

Sifalimumab (MEDI-545) is a fully human, immunoglobulin G1 K monoclonal antibody that binds to and neutralizes the majority of IFN-α subtypes⁴⁰. Sifalimumab is described U.S. Pat. No. 7,741,449, which is incorporated herein by reference in its entirety. The efficacy and safety of sifalimumab were assessed in a phase IIb, randomised, double-blind, placebo-controlled study (NCT01283139) of adults with moderate to severe active systemic lupus erythematosus (SLE). 431 patients were randomised and received monthly intravenous sifalimumab (200 mg, 600 mg or 1200 mg) or placebo in addition to standard-of-care medications. The primary efficacy end point was the percentage of patients achieving an SLE responder index response at week 52. Compared with placebo, a greater percentage of patients who received sifalimumab (all dosages) met the primary end point (placebo: 45.4%; 200 mg: 58.3%; 600 mg: 56.5%; 1200 mg 59.8%).

### 5.2.8. TNF Inhibitors

Tumor necrosis factor (TNF)-alpha inhibitors, including etanercept, infliximab, adalimumab, certolizumab pegol and golimumab, are biologic agents which are FDA-approved to treat a number of inflammatory conditions including ankylosing spondylitis, Crohn disease, hidradenitis suppurativa (A), juvenile idiopathic arthritis, plaque psoriasis, polyarticular juvenile idiopathic arthritis, psoriatic arthritis, rheumatoid arthritis, ulcerative colitis, and uveitis⁴¹.

### 5.2.8.1. Adalimumab

Adalimumab is a recombinant human IgG1 monoclonal antibody. Adalimumab is described in U.S. Pat. Nos. 8,197,813 and 8,372,401, which are incorporated herein by reference. Adalimumab is approved for treatment of Rheumatoid Arthritis (RA), Hidradenitis suppurativa; Ankylosing Spondylitis; Inflammatory Bowel Disease (IBD) (Crohn's disease, Ulcerative colitis), Plaque psoriasis and Juvenile idiopathic arthritis.

### 5.2.8.2. Etanercept

Etanercept (Enbrel, Elrelzi, Eticovo, YLB113) is described in U.S. Pat. No. 8,063,182, which is incorporated herein by reference. Etanercept is administered by subcutaneous injection. Dosage information is shown in

Table 5-10. Etanercept is approved for the treatment of Rheumatoid Arthritis (RA), Ankylosing Spondylitis, Plaque Psoriasis, Juvenile idiopathic arthritis and Plaque Psoriasis.

TABLE 5-10
Etanercept dosage and administration

| Route | Dose | Use/Indication |
|---|---|---|
| Subcutaneous | 50 mg once weekly | FDA approved, |
| | | Adult RA and PsA |
| Subcutaneous | 50 mg once weekly | Investigative, SLE |
| | 50 mg twice weekly for 3 | Adult PsO |
| | months followed by 50 mg | |
| | once weekly | |
| Subcutaneous | 0.8 mg/kg weekly, with a | |
| | maximum of 50 mg per week | |

### 5.2.8.3. Infliximab

Infliximab (Remicade, Avsola, Ixifi, Renflexis) is a TNF-alpha inhibitor. Infliximab is approved for the treatment of IBD (Chron's disease, RA, AS, Ulcerative colitis, and plaque psoriasis. Dosing information is shown in Table 5-11.

TABLE 5-11
Infliximab dosage and administration

| Route | Dose | Use/Indication |
|---|---|---|
| Intravenous | 5 mg/kg at 0, 2 and 6 weeks, then every 8 | FDA approved, Crohn's |
| | weeks. Some adult patients who initially respond | disease |
| | to treatment may benefit from increasing the | |
| | dose to 10 mg/kg every 8 weeks if they later lose | |
| | their response. | |
| Intravenous | 5 mg/kg at 0, 2 and 6 weeks, then every 8 weeks. | FDA approved, Pediatric |
| | | Crohn's Disease (≥6 years old) |
| Intravenous | 5 mg/kg at 0, 2 and 6 weeks, then every 8 weeks. | FDA approved, Ulcerative |
| | | Colitis |
| Intravenous | 5 mg/kg at 0, 2 and 6 weeks, then every 8 weeks | FDA approved, Pediatric |
| | | Ulcerative Colitis (≥ 6 years old) |
| Intravenous | In conjunction with methotrexate, 3 mg/kg at 0, 2 | FDA approved, Rheumatoid |
| | and 6 weeks, then every 8 weeks. Some patients | Arthritis |
| | may benefit from increasing the dose up to 10 | |
| | mg/kg every 8 weeks or treating as often as | |
| | every 4 weeks. | |
| Intravenous | 5 mg/kg at 0, 2 and 6 weeks, then every 6 weeks. | FDA approved, Ankylosing |
| | | Spondylitis |
| Intravenous | 5 mg/kg at 0, 2 and 6 weeks, then every 8 weeks | FDA approved, Psoriatic |
| | | Arthritis and Plaque Psoriasis |

### 5.2.9. Functional Variants

Functional variants of biologics are sequence variants that perform the same function as anifrolumab. Functional variants include biosimilars and interchangeable products. The terms biosimilar and interchangeable product are defined by the FDA and EMA. The term biosimilar refers to a biological product that is highly similar to an approved (e.g. FDA approved) biological product (reference product, e.g. anifrolumab) in terms of structure and has no clinically meaningful differences in terms of pharmacokinetics, safety and efficacy from the reference product. The presence of clinically meaningful differences of a biosimilar may be assessed in human pharmacokinetic (exposure) and pharmacodynamic (response) studies and an assessment of clinical immunogenicity. An interchangeable product is a biosimilar that is expected to produce the same clinical result as the reference product in any given patient.

### 5.3. Clinical Trials

### 5.3.1. Phase 2/Phase II/Pivotal Studies

Phase II studies gather preliminary data on effectiveness. In Phase 2 studies, researchers administer the drug to a group of patients with the disease or condition for which the drug is being developed. Typically involving a few hundred patients, these studies aren't large enough to show whether the drug will be beneficial. Instead, Phase 2 studies provide researchers with additional safety data. Researchers use these data to refine research questions, develop research methods, and design new Phase 3 research protocols.

### 5.3.2. Phase 3/Phase III/Pivotal Studies or Trials

Researchers design Phase 3 studies to demonstrate whether or not a product offers a treatment benefit to a specific population. Sometimes known as pivotal studies, these studies involve 300 to 3,000 participants. Phase 3 studies provide most of the safety data. In previous studies, it is possible that less common side effects might have gone undetected. Because these studies are larger and longer in duration, the results are more likely to show long-term or rare side effects. Regulatory bodies such as the EMA and FDA usually require a phase III clinical trial demonstrating that the product is safe and at least as effective (if not better) than available medications, before approving a new medication. Phase III clinical trials usually fail, even if they follow a successful a phase II clinical trial.

### 5.4. Formulations

Stable formulations suitable for administration to subjects and comprising anifrolumab are described in detail in U.S. Pat. No. 10,125,195 B1, which is incorporated herein in its in entirety.

The Examples that follow are illustrative of specific embodiments of the disclosure, and various uses thereof. They are set forth for explanatory purposes only and should not be construed as limiting the scope of the disclosure in any way.

### 5.5. Steroids

Oral corticosteroids (OCS, glucocorticoids) include prednisone, cortisone, hydrocortisone, methylprednisolone, prednisolone and triamcinolone. Examples of equivalent doses of oral prednisone are shown in Table 5-12.

TABLE 5-12
Examples of equivalent doses of oral prednisone

Oral Prednisone

| and Equivalents | Equivalent Dose | | | | | | | | | |
|---|---|---|---|---|---|---|---|---|---|---|
| Oral Prednisone | 7.5 | mg | 10 | mg | 20 | mg | 30 | mg | 40 | mg |
| Cortisone | 37.5 | mg | 50 | mg | 100 | mg | 150 | mg | 200 | mg |
| Hydrocortisone | 30 | mg | 40 | mg | 80 | mg | 120 | mg | 160 | mg |
| Methylprednisolone | 6 | mg | 8 | mg | 16 | mg | 24 | mg | 32 | mg |
| Prednisolone | 7.5 | mg | 10 | mg | 20 | mg | 30 | mg | 40 | mg |
| Triamcinolone | 6 | mg | 8 | mg | 16 | mg | 24 | mg | 32 | mg |

### 5.6. Delivery Device

As well as providing for subcutaneous administration of the antibody, the ability to self-administer (e.g. for home use) may further be enhanced by subcutaneous administration via an accessorized pre-filled syringe (APFS), an autoinjector (AI), or a combination thereof. Such devices have been found to be well-tolerated and reliable for administering subcutaneous doses of an antibody and provide further options for optimizing patient care. Indeed, such devices may reduce the burden of frequent clinic visits for patients. An example of a suitable APFS device is described in Ferguson et. al.⁴², which is incorporated herein by reference in its entirety.

The dose elucidated by the inventors provides yet advantages in the context of APFS-administration, as an APFS device typically administers a maximal volume of 1 ml. A dose in the range of >105 mg to <155 mg can be readily accommodated by a volume of ˜0.8 ml, such that the dose(s) of the present invention are uniquely suited to APFS and Al administration. For comparison, due to viscosity of the anifrolumab, larger doses (particularly doses of >150 mg) would need to be administered within a volume of >1 ml, requiring at least two SC injections, which is inconvenient for the patient, and would require a plurality of pre-filled devices.

The delivery device may be single use, disposable system that is designed to enable manual, SC administration of the dose.

### 5.7. Dosage Forms

A unit dose (also referred to as a unit dose form, a pharmaceutical unit dose or a pharmaceutical unit dose form) is a dose formed from a single unit. A unit dose (unit dose form) is suitable for administration to a subject in a single administration step. A unit dose (unit dose form) may be packaged in a single-unit container, for example a single-use pre-filled syringe or autoinjector. Unit doses provide the advantage that they can be ordered, packaged, handled and administered as single dose units containing a pre-determined amount of a drug. Unit doses decrease administration errors and reduce waste.

### 5.8. End Points

### 5.8.1. Patient Reported Outcomes

Physician Global Assessment (PGA and MDGA) of Disease Activity refers to an assessment wherein a physician evaluates the status of a subject's psoriatic arthritis (PsA) by means of a visual analog scale (VAS). The subject is assessed according to how their current arthritis is. The VAS is anchored with verbal descriptors of “very good” to “very poor.”

### 5.8.2. BILAG-2004 (British Isles Lupus Assessment Group-2004)

The BILAG-2004 is a translational index with 9 organ systems (General, Mucocutaneous, Neuropsychiatric, Musculoskeletal, Cardiorespiratory, Gastrointestinal, Ophthalmic, Renal and Haematology) that is able to capture changing severity of clinical manifestations. It has ordinal scales by design and does not have a global score; rather it records disease activity across the different organ systems at a glance by comparing the immediate past 4 weeks to the 4 weeks preceding them. It is based on the principle of physicians' intention to treat and categorizes disease activity into 5 different levels from A to E:
Grade A represents very active disease requiring immunosuppressive drugs and/or a prednisone dose of >20 mg/day or equivalent
Grade B represents moderate disease activity requiring a lower dose of corticosteroids, topical steroids, topical immunosuppressives, antimalarials, or NSAIDs
Grade C indicates mild stable disease.
Grade D implies no disease activity but the system has previously been affected
Grade E indicates no current or previous disease activity

Although the BILAG-2004 was developed based on the principle of intention to treat, the treatment has no bearing on the scoring index. Only the presence of active manifestations influences the scoring.

BILAG-defined improvement in mucocutaneous or musculoskeletal organ systems were representative of rash or arthritis, respectively.

### 5.8.3. BICLA (BILAG-Based Composite Lupus Assessment)

BICLA is a composite index that was originally derived by expert consensus of disease activity indices. BICLA response is defined as (1) at least one gradation of improvement in baseline BILAG scores in all body systems with moderate or severe disease activity at entry (e.g., all A (severe disease) scores falling to B (moderate), C (mild), or D (no activity) and all B scores falling to C or D); (2) no new BILAG A or more than one new BILAG B scores; (3) no worsening of total SLEDAI score from baseline; (4) no significant deterioration (≤10%) in physicians global assessment; and (5) no treatment failure (initiation of non-protocol treatment).

Particularly, a subject is a BICLA responder if the following criteria are met:
a) Reduction of all baseline BILAG-2004 A to B/C/D and baseline BILAG-2004 B to C/D, and no BILAG-2004 worsening in other organ systems, as defined by 1 new BILAG-2004 A or more than 1 new BILAG-2004 B item;
b) No worsening from baseline in SLEDAI-2K as defined as an increase from baseline of >0 points in SLEDAI-2K;
c) No worsening from baseline in the subjects' lupus disease activity defined by an increase ≥0.30 points on a 3-point PGA VAS;
d) No discontinuation of investigational product or use of restricted medications beyond the protocol-allowed threshold before assessment

### 5.8.4. CLASI (Cutaneous Lupus Erythematosus Disease Area and Severity Index Inflammatory Disease Activity)

The Cutaneous Lupus Erythematosus Disease Area and Severity Index (CLASI) was developed in 2005 as a means of specifically tracking cutaneous activity and damage in patients with CLE²¹. The CLASI is a simple, single-page tool that separately quantifies skin disease activity and damage in each part of the body⁴³. The CLASI features a skin activity summary score (CLASI-A) and damage summary score (CLASI-D). This index has a high inter-rater and intra-rater reliability and is responsive to change when used in adults with CLE and SLE. CLASI activity score correlates with the severity of disease: mild, moderate, and severe disease corresponded with CLASI activity score ranges of 0-9 (sensitivity 93%, specificity 78%), 10-20, and 21-70 (sensitivity 80%, specificity 95%), respectively (Table 5-13).

TABLE 5-13
CLE disease severity based on the CLASI activity score

| CLE disease | CLASI activity score range |
|---|---|
| Mild | 0-9 |
| Moderate | 10-20 |
| Severe | 21-70 |

The Cutaneous Lupus Erythematosus Disease Area and Severity Index (CLASI) quantifies disease activity and damage in cutaneous lupus erythematosus. It can distinguish between different response levels of treatment, e.g., it is able to detect a specific percentage reduction in activity score from baseline, or can be reported by a mean/median score. Particularly, the CLASI is a validated index used for assessing the cutaneous lesions of lupus and consists of 2 separate scores: the first summarizes the inflammatory activity of the disease; the second is a measure of the damage done by the disease. The activity score takes into account erythema, scale/hypertrophy, mucous membrane lesions, recent hair loss, and nonscarring alopecia. The damage score represents dyspigmentation, scarring/atrophy/panniculitis, and scarring of the scalp. Subjects are asked if their dyspigmentation lasted 12 months or longer, in which case the dyspigmentation score is doubled. Each of the above parameters is measured in 13 different anatomical locations, included specifically because they are most often involved in cutaneous lupus erythematosus (CLE). The most severe lesion in each area is measured.

Modified CLASI (mCLASI) is defined as the activity portions of CLASI that describe skin erythema, scale/hypertrophy, and inflammation of the scalp. Activity of oral ulcers and alopecia without scalp inflammation are excluded from the mCLASI analysis, as are all measures of damage. Clinically meaningful improvement in rash, as measured using mCLASI, is defined by ≥50% decrease in baseline activity score.

### 5.8.5. SRI (Systemic Lupus Erythematosus Responder Index of ≥4)

A subject achieves SRI (4) if all of the following criteria are met:
Reduction from baseline of >4 points in the SLEDAI-2K;
No new organ system affected as defined by 1 or more BILAG-2004 A or 2 or more
BILAG-2004 B items compared to baseline using BILAG-2004;
No worsening from baseline in the subjects' lupus disease activity defined by an increase ≥0.30 points on a 3-point PGA VAS.

SRI (X) (X=5, 6, 7, or 8) is defined by the proportion of subjects who meet the following criteria:
Reduction from baseline of ≥X points in the SLEDAI-2K;
No new organ systems affected as defined by 1 or more BILAG-2004 A or 2 or. more BILAG-2004 B items compared to baseline using BILAG-2004;
No worsening from baseline in the subjects' lupus disease activity defined by an
increase ≥0.30 points on a 3-point PGA VAS

### 5.8.6. SLEDAI-2K (Systemic Lupus Erythematosus Disease Activity Index 2000)

The SLEDAI-2K disease activity index consists of a list of organ manifestations, each with a definition. A certified Investigator or designated physician will complete the SLEDAI-2K assessment and decide whether each manifestation is “present” or “absent” in the last 4 weeks. The assessment also includes the collection of blood and urine for assessment of the laboratory categories of the SLEDAI-2K.

The SLEDAI-2K assessment consists of 24 lupus-related items. It is a weighted instrument, in which descriptors are multiplied by a particular organ's “weight”. For example, renal descriptors are multiplied by 4 and central nervous descriptors by 8 and these weighted organ manifestations are totaled into the final score. The SLEDAI-2K score range is 0 to 105 points with 0 indicating inactive disease. The SLEDAI-2K scores are valid, reliable, and sensitive clinical assessments of lupus disease activity. The SLEDAI-2K calculated using a timeframe of 30 days prior to a visit for clinical and laboratory values has been shown to be similar to the SLEDAI-2K with a 10-day window⁴⁴.

SLEDAI-2K-defined resolution of rash is defined as a score of 0 at Week 52 for those with a score ≥2 for rash at baseline.

### 5.8.7. Tender and Swollen Joints

The swollen and tender joint count may be based on left and right shoulder, elbow, wrist, metacarpophalangeal (MCP) 1, MCP2, MCP3, MCP4, MCP5, proximal interphalangeal (PIP) 1, PIP2, PIP3, PIP4, PIP5 joints of the upper extremities and left and right knee of the lower extremities. An active joint for the joint count assessment may be defined as a joint with tenderness and swelling.

### 5.8.8. Systemic Lupus International Collaborating Clinics/American College of Rheumatology Damage Index (SDI)

SDI is used to measure organ damage in SLE patients. Particularly, the SDI was been developed to assess irreversible damage in SLE subjects independently of its cause (SLE activity, therapy, comorbidities) but occurring after disease onset. Damage, i.e., irreversible impairment since onset of SLE is usually defined as a clinical feature that has to be continuously present for at least 6 months to score. In addition some irreversible events such as MI or a cerebrovascular accident score as damage on their occurrence. Briefly, damage is defined for 12 organ systems; peripheral vascular, ocular, neuropsychiatric, renal, pulmonary, cardiovascular, gastrointestinal, musculoskeletal, skin, endocrine (diabetes), gonadal, and malignancies. Damage over time can be stable or increase, to a maximum of 47 points, however there should be no decrease in the number of points⁴⁵.

### 5.9. PK/PD

Plasma levels obtainable by SC administration and IV administration may be compared on the basis of a plasma drug concentration-time curve (AUC), which reflects the body exposure to the antibody after administration of a dose of the drug. For example, during a clinical study, the patient's plasma drug concentration-time profile can be plotted by measuring the plasma concentration at several time points. Where an in silico modelling approach is employed, plasma drug concentration-time for any given dose may be predicted. The AUC (area under the curve) can then be calculated by integration of the plasma drug concentration-time curve. Suitable methodology is described in Tummala et. al.²⁶, which is incorporated herein by reference in its entirety. In the Examples described herein, PK parameters were calculated by non-compartmental analysis with Phoenix WinNonlin V/6.2 (Certara, Inc., Princeton, New Jersey, USA) and included the area under the serum concentration-time curve (AUC), clearance (CL, CL/F), maximum serum concentration (Cₘₐₓ) and time to reach maximum serum concentration (tₘₐₓ). All data were analyzed with SAS System V.9.2 (SAS Institute, Inc., Cary, NC, USA).

Conveniently, a ratio of the AUC obtainable with SC administration to the AUC obtainable by IV administration (AUC_{SC}/AUC_{IV}) may be calculated, providing a numerical comparison of bioavailability provided by the dosage routes. Reference to the “AUC Ratio” herein means the AUC_{SC}/AUC_{IV} ratio. To provide statistical robustness, the AUC ratio is preferably a mean, median or mode (for example, a mean) value calculated from a plurality of repeat experiments (or computational simulations). This approach is demonstrated with reference to the Examples. The mean, median or mode (preferably mean) may be derived by pooling data obtained from multiple patients (or multiple computational simulations). Thus, the AUC Ratio may reflect the mean, median or mode (preferably mean) AUC in multiple patients.

### 5.10. Pharmacokinetics Glossary

Area under the curve (AUC): Area under the plasma drug concentration versus time curve, which serves as a measure of drug exposure.

Cₐᵥₑ: Steady-state average concentration.

Cₘₐₓ: The maximum (or peak) concentration of the drug in the plasma.

Cₘᵢₙ: Minimum plasma drug concentration.

C_{trough}: the concentration of drug in plasma at steady state immediately prior to the administration of a next dose. Trough plasma concentration (measured concentration at the end of a dosing interval at steady state [taken directly before next administration]).

LLOQ: The lower limit of quantitation, the lowest amount of an analyte in a sample that can be quantitatively determined with suitable precision and accuracy.

Linear pharmacokinetics: When the concentration of the drug in the blood or plasma increases proportionally with the increasing dose, and the rate of elimination is proportional to the concentration, the drug is said to exhibit linear pharmacokinetics. The clearance and volume of distribution of these drugs are dose-independent.

Nonlinear pharmacokinetics: As opposed to linear pharmacokinetics, the concentration of the drug in the blood or plasma does not increase proportionally with the increasing dose. The clearance and volume of distribution of these may vary depending on the administered dose. Nonlinearity may be associated with any component of the absorption, distribution, and/or elimination processes.

### 5.11. Type I IFN Gene Signature (IFNGS)

Type I IFN is considered to play a central role SLE disease pathogenesis and inhibition of this pathway is targeted by anifrolumab. To understand the relationship between type I IFN expression and response to anti-IFN therapy, it is necessary to know if a subject's disease is driven by type I IFN activation. However, direct measurement of type I IFN remains a challenge. As such, a transcript-based marker was developed to evaluate the effect of over expression of the target protein on a specific set of mRNA markers.

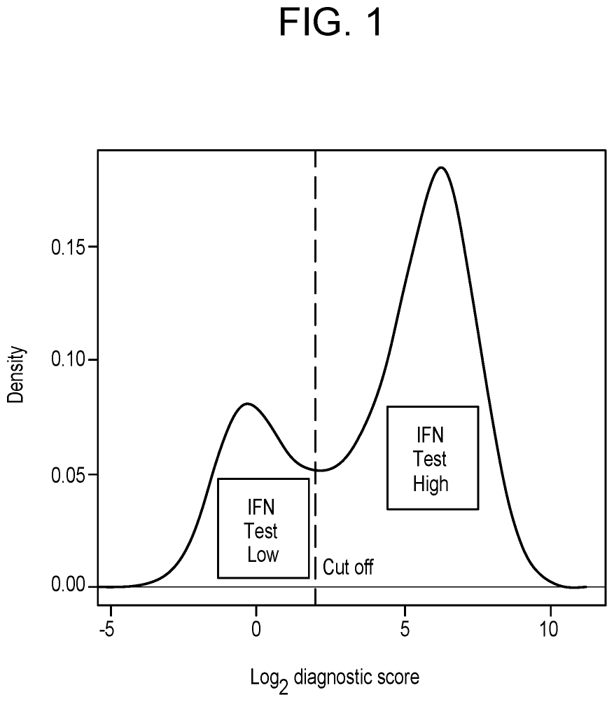

The expression of these markers is easily detected in whole blood and demonstrates a correlation with expression in diseased tissue such as skin in SLE. The bimodal distribution of the transcript scores for SLE subjects supports defining an IFN test high and low subpopulation (FIG. 1). The type I IFN test is described in WO2011028933 A1, which is incorporated herein by reference in its entirety. The type I IFN gene signature may be used to identify a subject has a type I IFN gene signature (IFNGS)-test high patient or an IFNGS-test low patient. The IFNGS test measures expression of the genes IFI27, IFI44, IFI44L, and RSAD2 compared with 3 reference genes; 18S, ACTB and GAPDH in the whole blood of the subject. The result of the test is a score that is compared with a pre-established cut-off that classifies patients into 2 groups with low or high levels of IFN inducible gene expression (FIG. 1).

The expression of the genes may be measured by RT-PCR. Suitable primers and probes for detection of the genes may be found in WO2011028933. A suitable kit for measuring gene expression for the IFNGS test is the QIAGEN Therascreen® IFIGx RGQ RT-PCR kit (IFIGx kit), as described in Brohawn et al.⁴⁶, which is incorporated herein by reference in its entirety.

### 5.12. Standard of Care (SOC)

Standard of care includes⁴⁷:
A dose of oral prednisone (≤40 mg/day) or prednisone equivalent dose
Azathioprine≤200 mg/day
Antimalarials: chloroquine, hydroxychloroquine (200-400 mg daily), quinacrine (50-100 mg daily)
Mycophenolate mofetil≤2 g/day or mycophenolic acid≤1.44 g/day
Oral, subcutaneous (SC), or intramuscular methotrexate≤25 mg/week
Cyclosporine (75-100 mg daily)
Leflunomide (10-20 mg daily)
Mizoribine≤150 mg/day
NSIADs, e.g. naproxen sodium (Aleve), ibuprofen.

### 6. Example 1: MUSE, ClinicalTrial.gov Identifier: NCT01438489

MUSE was a Phase 2, multinational, multicentre, randomized, double-blind, placebo controlled, parallel-group study to evaluate the efficacy and safety of 2 intravenous (IV) treatment regimens in adult participants with chronic, moderately-to-severely active SLE with an inadequate response to standard of care (SOC) SLE. The investigational product (anifrolumab or placebo) was administered as a fixed dose every 4 weeks (28 days) for a total of 13 doses.

MUSE is described in further detail in Furie et al. 201715, which is incorporated herein by reference in its entirety.

### 7. Example 2: TULIP I and II, ClinicalTrial.gov Identifiers: NCT02446912 and NCT02446899

TULIP I and TULIP II were Phase 3, multicentre, multinational, randomised, double-blind, placebo-controlled studies to evaluate the efficacy and safety of an intravenous (IV) treatment regimen of two doses of anifrolumab versus placebo in subjects with moderately to severely active, autoantibody-positive systemic lupus erythematosus (SLE) while receiving standard of care (SOC) treatment.

### 7.1.1. Restricted Medications

If a subject received 1 of the following, the subject was considered a non-responder. Sulfasalazine; Danazol; Dapsone; Azathioprine >200 mg/day or at a daily dose greater than that at Week 0 (Day 1); Mycophenolate mofetil >2.0 g/day or mycophenolic acid >1.44 g/day or at a daily; dose greater than that at Week 0 (Day 1); Oral, SC, or intramuscular methotrexate >25 mg/week or at a daily dose greater than that at Week 0 (Day 1); Mizoribine >150 mg/day or at a daily dose greater than that at Week 0 (Day 1); Any change in route of administration of oral, SC, or intramuscular methotrexate; Intravenous corticosteroids >40 mg/day but ≤1 gm/day methylprednisolone or equivalent; Intramuscular corticosteroids >80 mg/day methylprednisolone or equivalent; Subcutaneous or intramuscular corticosteroid precursors; Treatment with OCS >40 mg/day prednisone or equivalent; Treatment with OCS above Day 1 dose for a dosing period >14 days; Corticosteroids with a long biologic half-life (e.g., dexamethasone, betamethasone); Other immunosuppressants including but not limited to calcineurin inhibitors (e.g., cyclosporine, tacrolimus [including topical]) or leflunomide. Cyclosporine eye drops were acceptable for use in the study.

TULIP I is described in further detail in Furie et al. 201916, which is incorporated herein by reference in its entirety. The results of TULIP II are presented in Morand et al. 202014, herein incorporated by reference in its entirety.

### 8. Example 3: Efficacy of Anifrolumab in Patients With SLE Previously Treated With Biologics: Post Hoc Analysis of Data from 2 Phase 3 Trials

### 8.1. Background

Anifrolumab, a monoclonal antibody that binds the type I IFN receptor, was efficacious and well tolerated in adult patients with moderate to severe SLE despite standard therapy in the phase 3 TULIP-1 and TULIP-2 trials. Patients with SLE who have previously been treated with biologics represent an important patient population with limited treatment options. The inventors investigated whether prior exposure to immunomodulatory biologics impacted anifrolumab efficacy and safety in TULIP-1 and TULIP-2 pooled data.

### 8.2. Methods

This analysis included patients who received intravenous anifrolumab 300 mg or placebo every 4 weeks for 48 weeks in the 52-week TULIP-1 (NCT02446912) and TULIP-2 (NCT02446899) trials, for which eligible patients met the ACR SLE criteria, had moderate to severe SLE, and were permitted prior biologic use with a 3-6 month washout period, regardless of the reason for cessation. Patients were split into biologic-experienced or biologic-naïve subgroups (≥1 or 0 previous biologic immunomodulators, respectively). Baseline SLE disease characteristics, efficacy, and safety were compared across subgroups. Efficacy measures included BILAG-based Combined Lupus Assessment (BICLA) response at Week (W) 52; SLE Responder Index of ≥4 (SRI [4]) response at W52; sustained oral glucocorticoid (GC) taper (≤7.5 mg/day prednisone equivalent from W40-52 if ≥10 mg/day at baseline); and annualized flare rate through W52. Binary endpoints and safety were analyzed with a Cochran-Mantel-Haenszel approach controlling for randomization stratification factors and study. Annualized flare rate was analyzed with a negative binomial regression model with treatment, randomization stratification factors, and study as covariates.

### 8.3. Results

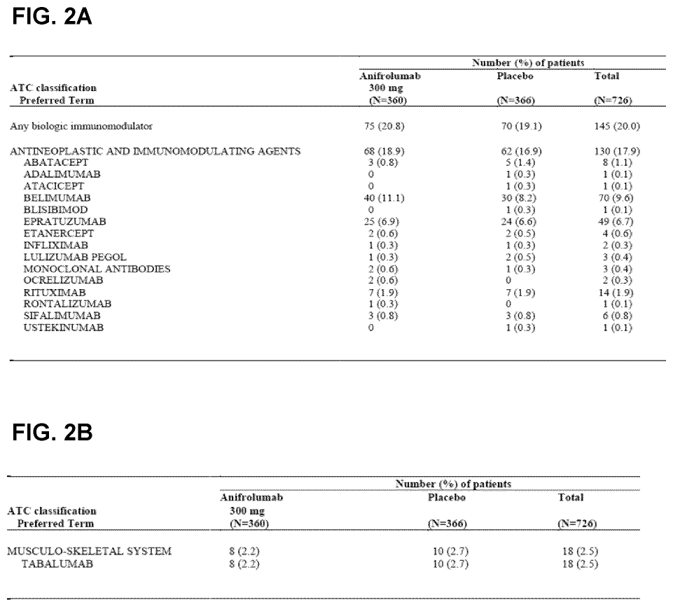

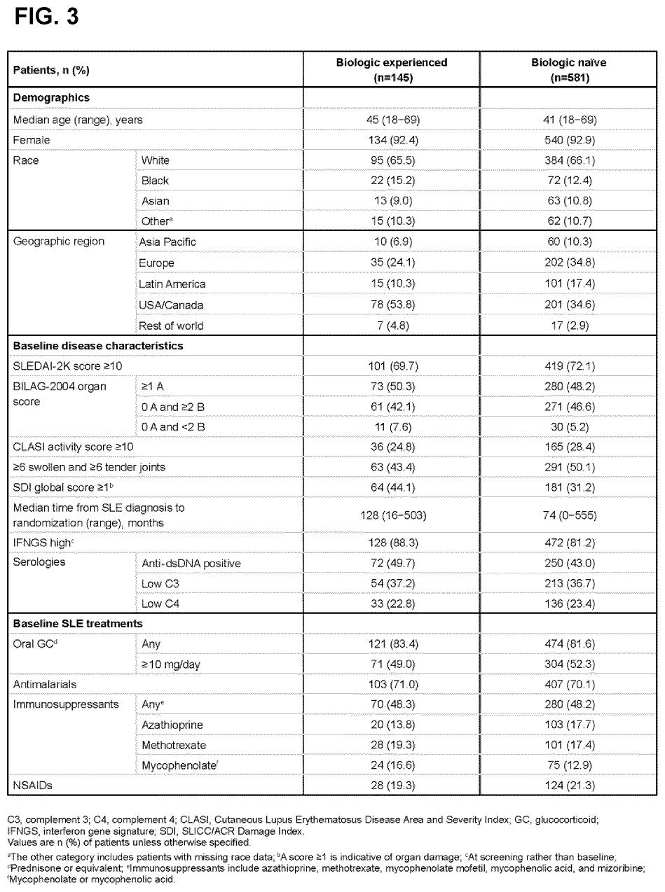

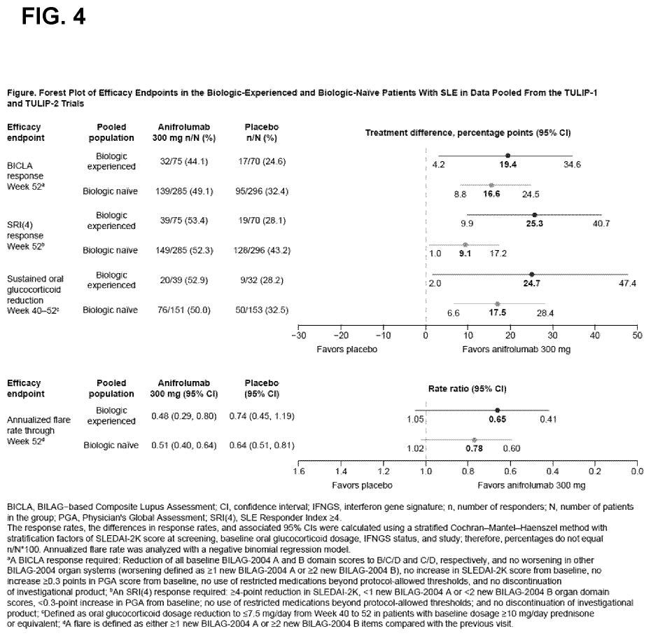

There were 145 biologic-experienced patients (anifrolumab [n=75]; placebo [n=70]), and 581 biologic-naïve patients (anifrolumab [n=285]; placebo [n=296]). Most previous biologic use was with belimumab (n=70), epratuzumab (n=49), tabalumab (n=18), or rituximab (n=14) (FIG. 2A, FIG. 2B). Baseline demographics, disease characteristics, and non-biologic SLE treatments were generally similar between groups. However, compared with biologic-naïve patients, biologic-experienced patients had longer times from SLE diagnosis and were more likely to be from North America, have SLICC/ACR Damage Index organ damage score ≥1, anti-dsDNA antibodies, and high IFN gene signatures (FIG. 3). There were lower placebo responses (potentially more refractory disease) in biologic-experienced vs biologic-naïve patients (FIG. 4). Anifrolumab was associated with greater treatment differences over placebo (Δ) in biologic-experienced vs biologic-naïve patients across multiple endpoints, including BICLA response (Δ=19.4 vs Δ=16.6), SRI (4) response (Δ=25.3 vs Δ=9.1), and oral GC tapers (Δ=24.7 vs Δ=17.5) (FIG. 4).

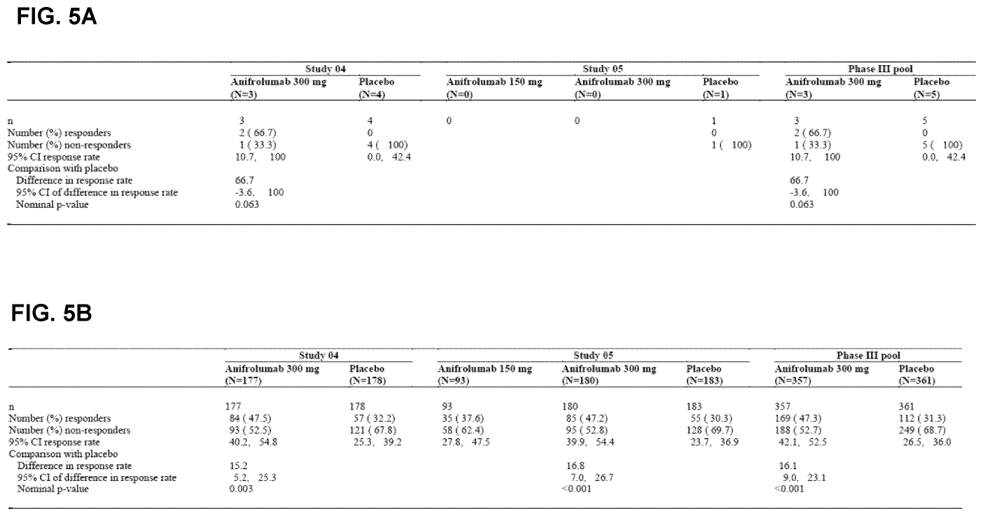

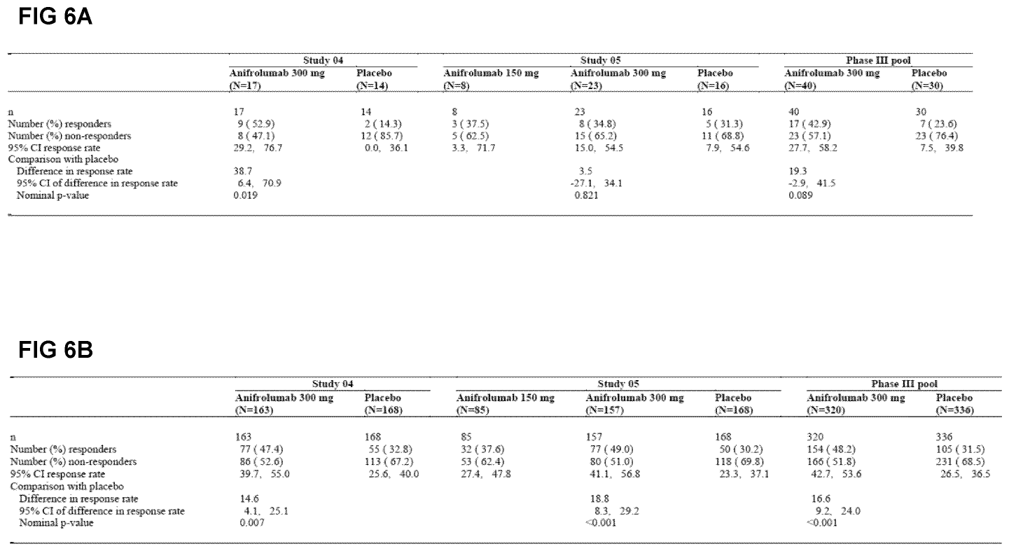

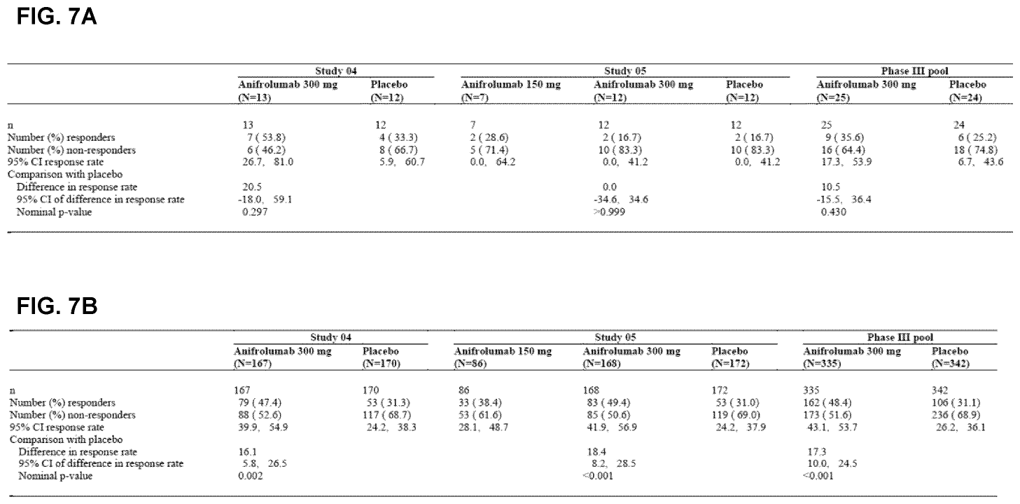

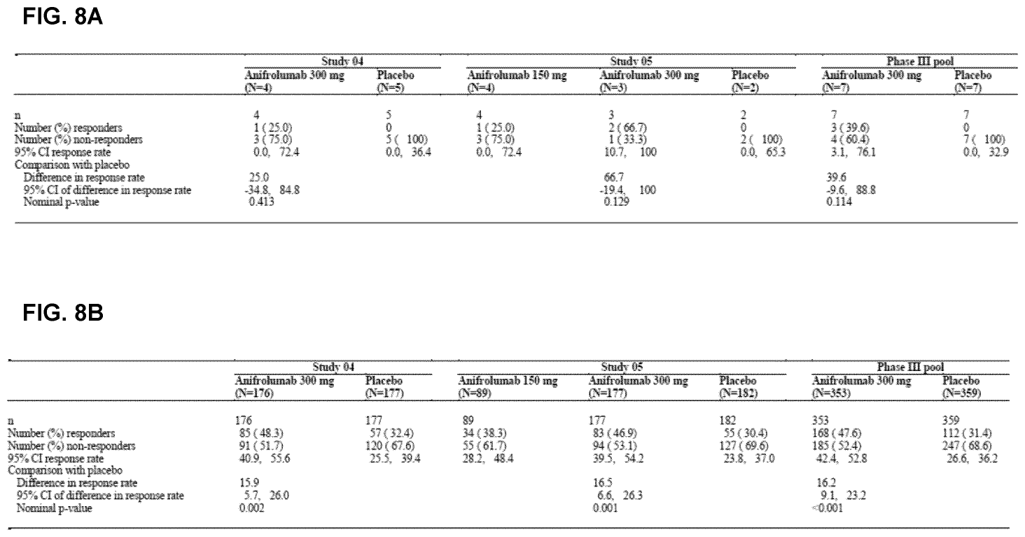

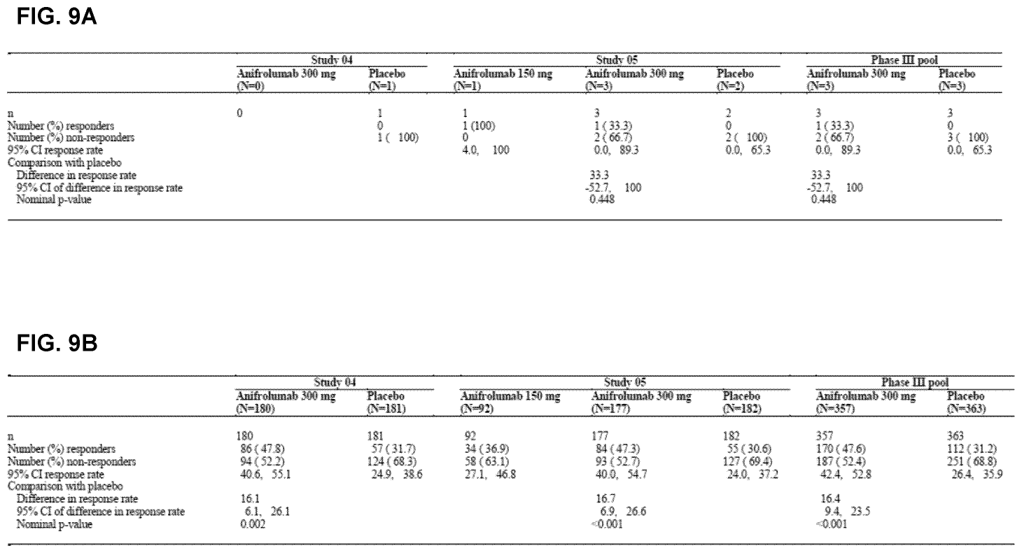

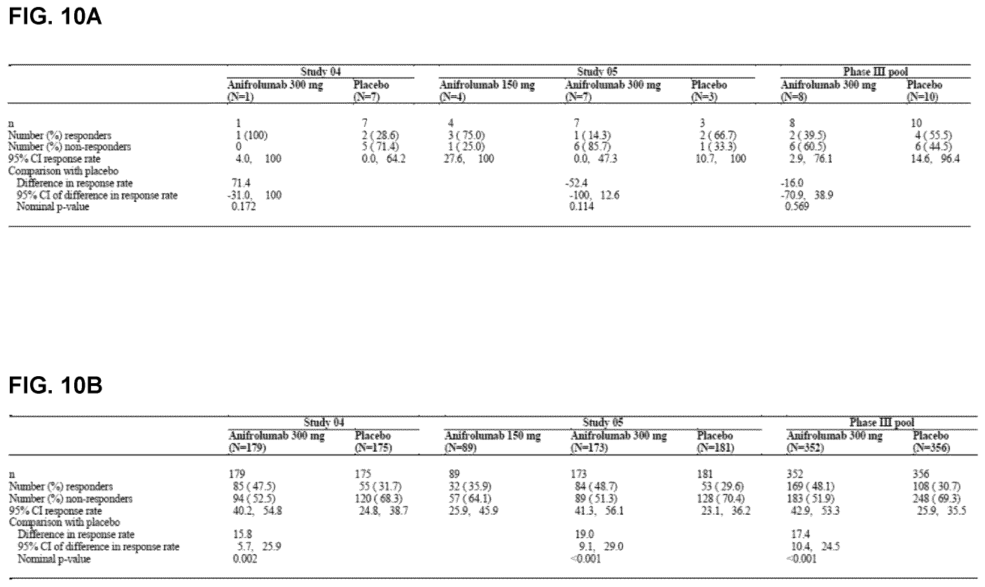

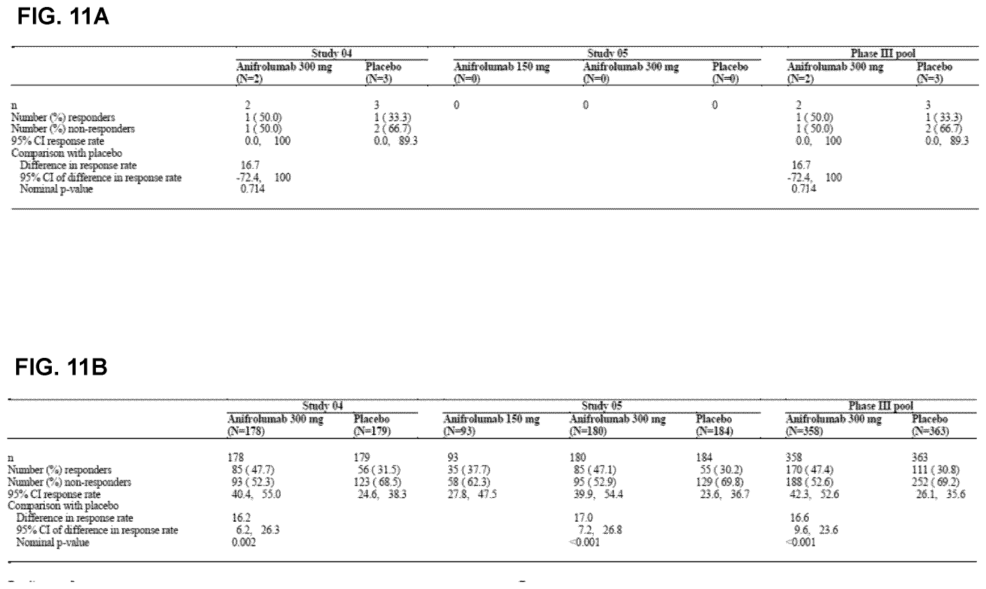

Anifrolumab was associated with greater treatment differences (BICLA response) over placebo (Δ) in patients with previous use of abatacept (FIG. 5A) vs patients without previous use of abatacept (4=66.7 vs Δ=16.1) (FIG. 5B). Anifrolumab was associated with greater treatment differences (BICLA response) over placebo (Δ) in patients with previous use of belimumab (FIG. 6A) vs patients without previous use of belimumab (Δ=19.3 vs Δ=16.6) (FIG. 6B). Anifrolumab was not associated with greater treatment differences (BICLA response) over placebo (Δ) in patients with previous use of epratuzumab (FIG. 7A) vs patients without previous use of epratuzumab (Δ=10.5 vs Δ=17.3) (FIG. 7B). Anifrolumab was associated with greater treatment differences (BICLA response) over placebo (Δ) in patients with previous use of rituximab (FIG. 8A) vs patients without previous use of rituximab (Δ=39.6 vs Δ=16.2) (FIG. 8B). Anifrolumab was associated with greater treatment differences (BICLA response) over placebo (Δ) in patients with previous use of sifalimumab (FIG. 9A) vs patients without previous use of sifalimumab (4=33.3 vs Δ=16.2) (FIG. 9B). Anifrolumab was not associated with greater treatment differences (BICLA response) over placebo (Δ) in patients with previous use of tabalumab or TNF inhibitors (FIG. 10A and FIG. 11A) vs patients without previous use of tabalumab or TNF inhibitors (Δ=39.6 vs Δ=16.2: (Δ=16.7 vs Δ=16.6) (FIG. 10B and FIG. 11B).

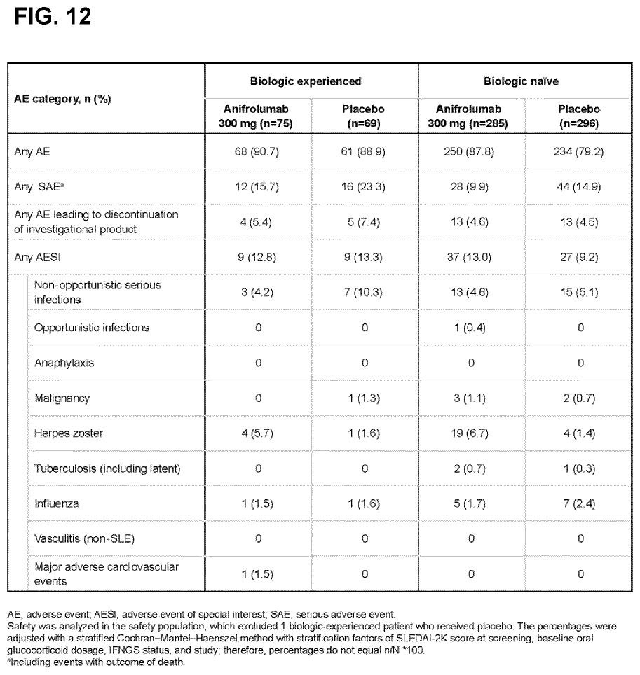

Incidence of serious adverse events was higher in biologic-experienced vs biologic-naïve patients with anifrolumab and placebo (FIG. 12). In biologic-experienced and biologic-naïve patients, herpes zoster incidence was higher with anifrolumab vs placebo.

### 8.4. Conclusion

Patients with SLE who have previously received biologics have a high unmet need and limited treatment options. Anifrolumab was associated with greater treatment differences over placebo in biologic-experienced vs biologic-naïve patients across multiple endpoints. Anifrolumab was associated with greater treatment differences over placebo in patients with prior use of abatacept, belimumab, rituximab, or sifalimumab. Anifrolumab was particularly effective in patients with prior use of belimumab. These data support the use of anifrolumab to treat SLE in patients with prior biologic use, particular prior abatacept, belimumab, rituximab, or sifalimumab use, most particularly with prior belimumab use.

### 9. Example 3: Subcutaneous Administration of Anifrolumab

### 9.1. Phase I Study MI-CP180 of IV Anifrolumab in Patients with SSc

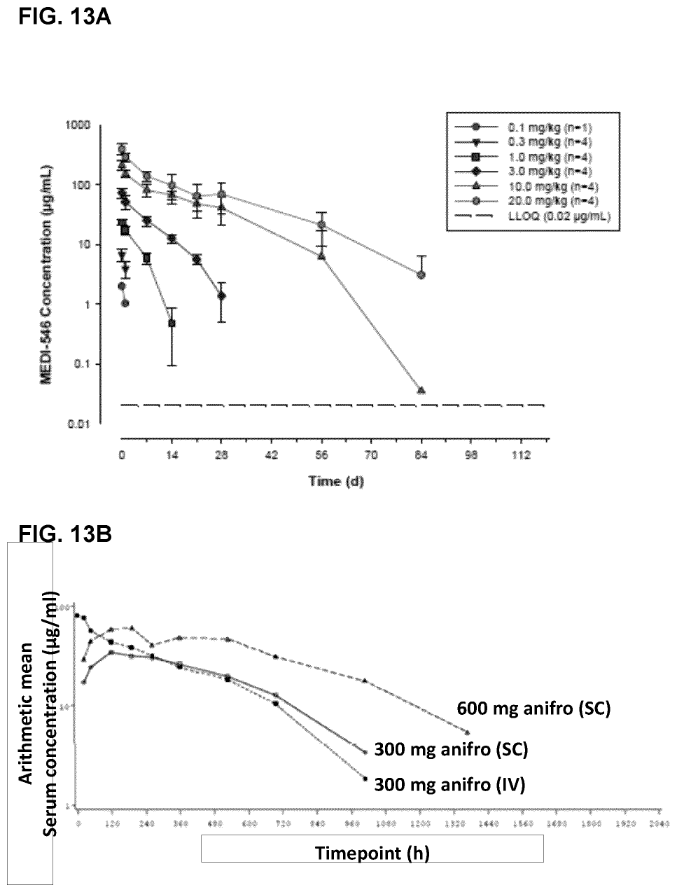

Mean anifrolumab serum concentrations after a single-dose administration based on body weight are presented in FIG. 13A. After a single-dose administration, anifrolumab exhibited nonlinear-linear PK at lower dose levels (<10.0 mg/kg) in both IFNGS high and IFNGS low patients. A dose-proportional increase in Cₘₐₓ was observed, but an increase in AUC was more than dose proportional between 0.1 and 10.0 mg/kg. Anifrolumab t1/2 was more prolonged in higher dose cohorts. At the highest dose level investigated (20.0 mg/kg), the terminal t1/2 was approximately 12 days.

### 9.2. Phase I of IV and SC Anifrolumab in Healthy Volunteers (Study 06)

In this Phase I randomized, placebo-controlled study, 30 healthy adults were assigned to three treatment cohorts (anifrolumab 300 mg SC (n=6), anifrolumab 300 mg intravenous (n=6), anifrolumab 600 mg SC (n=6)) and placebo (n=4/cohort). After SC administration, exposure to anifrolumab increased dose proportionally from 300 mg to 600 mg based on area under the serum concentration-time curve. Arithmetic mean serum anifrolumab concentration-time profiles following single IV and SC administration are shown in FIG. 13B. As reported in Tummala et al. 201826, which is incorporated herein by reference in its entirety, this study estimated the bioavailability to anifrolumab in healthy volunteers to be 87% of the intravenous exposure.

### 9.3. Phase II of SC Anifrolumab in SLE Patients (Study 08)

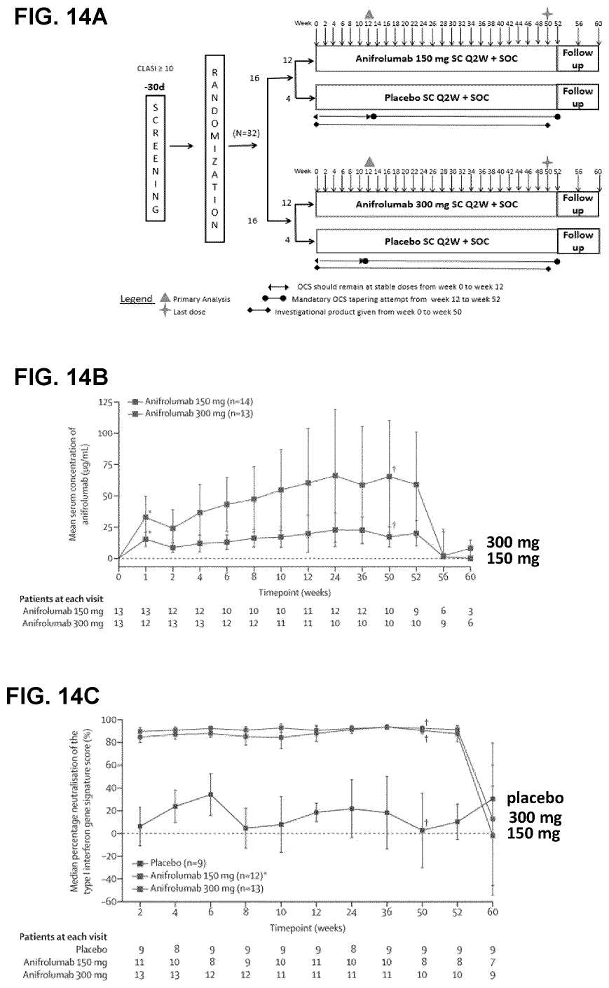

This study was designed to characterize the pharmacokinetics and pharmacodynamics of subcutaneously administered anifrolumab (FIG. 14A).

The study explored the clinical pharmacology, safety, and exploratory efficacy of subcutaneous anifrolumab. Pharmacokinetics in Study 08 were consistent with the high bioavailability in Study 06 (healthy volunteers) and high CL in IFNGS high patients with SLE. Anifrolumab, administered subcutaneously every 2 weeks to patients with SLE and moderate-to-severe skin manifestations had non-linear pharmacokinetics that were more than dose proportional, and neutralized the type I interferon gene signature in a dose-dependent manner (FIG. 14B and FIG. 14C). In particular, 150 mg or 300 mg of subcutaneous anifrolumab administered every 2 weeks for 50 weeks had non-linear pharmacokinetics, whereby C_{trough} concentrations were more than dose proportional. The number of adverse events with subcutaneous anifrolumab was similar to the numbers observed following intravenous administration in larger studies of patients with SLE.

The results of Study 08 are fully described in Bruce et al.⁴⁸, which is incorporated herein by reference in its entirety.

Study 08 was limited by small samples sizes, and no conclusions could be drawn about the biological effects of the study drug (e.g., on complement C3 or C4 concentrations) or its clinical efficacy. The inclusion of only patients with high type I interferon gene signatures and active skin disease also limited the generalizability of the study to patients with similar disease characteristics. The study was further limited by the increasing frequency of missing values with time.

### 9.4. Conclusion

The PK of anifrolumab consistently exhibited target mediated drug disposition where the concentrations or exposures decreased more than dose-proportional at lower dose levels. High bioavailability of anifrolumab administered via SC injection was observed in Study 06 (healthy volunteers); the ratio of the AUC of anifrolumab SC to anifrolumab IV under 300 mg was approximately 87%.

### 10. Example 4: Determination of the Optimal Subcutaneous Unit Dose

### 10.1. Aim

In order to detect an optimal dosage regimen for subcutaneous administration of anifrolumab, the inventors developed a population PK and a PK/PD model, designed to utilize existing human clinical trial. The PK data from phase III Studies 04 and 05 and phase II Study 1013 were used to assist the development of the population PK model.

An initial goal of the inventors was to detect a subcutaneous dose providing an equivalent exposure as a standard 300 mg IV (Q4W) dose, while concomitantly allowing more regular dosing that could be provided in a lower volume. This was based on the understanding that 300 mg IV Q4W provides optimal clinical PK profiles and clinical efficacy (e.g. in terms of achieving BICLA response) as reported e.g. in Furie et. al. 201715 which is incorporated herein by reference in its entirety, and summarized in Examples 3 and 4.

### 10.2. Results

### 10.2.1. Initial Selection of the Subcutaneous Dose for Anifrolumab

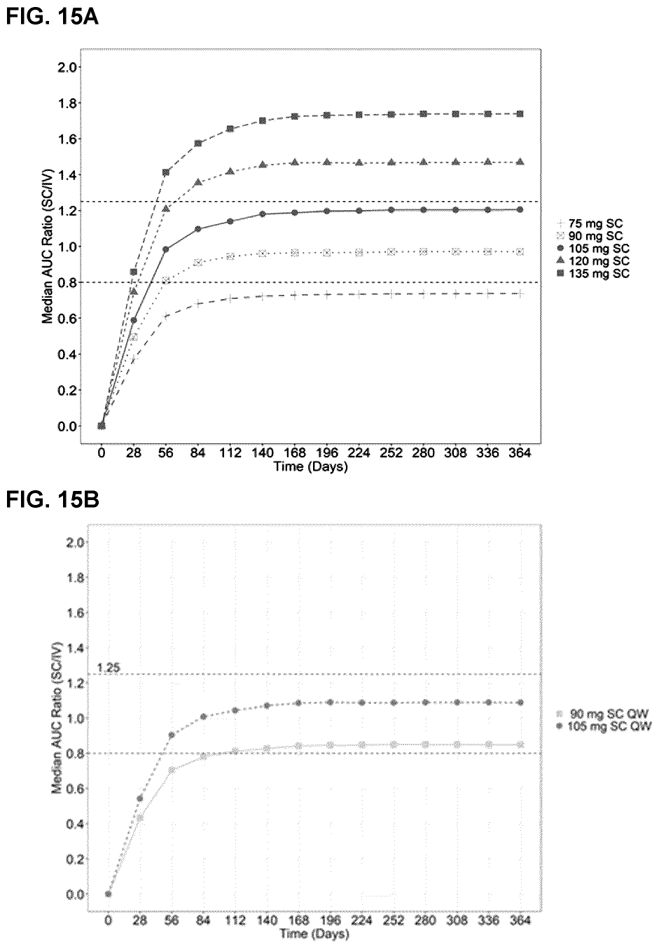

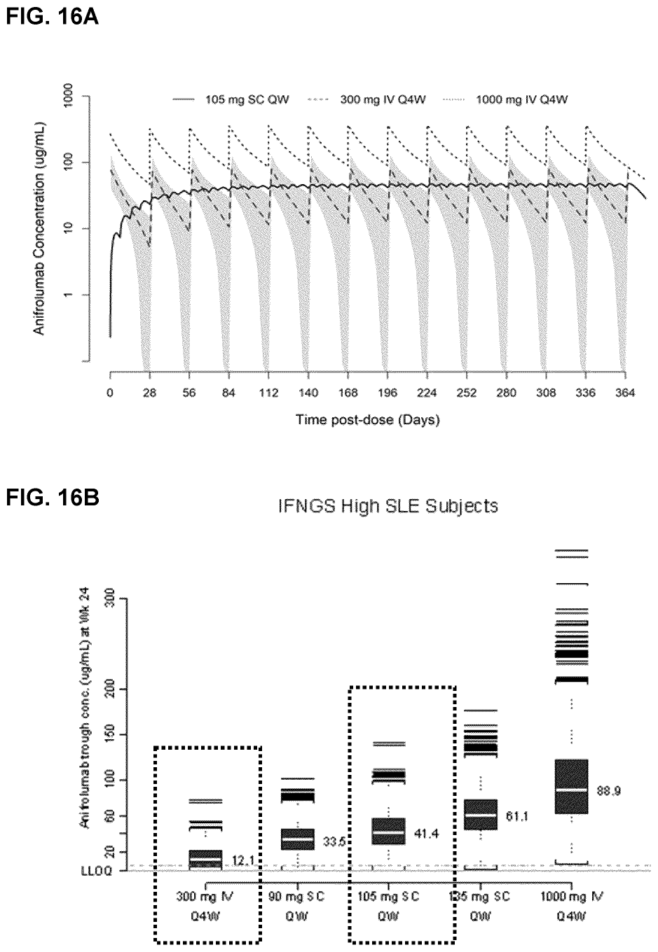

In an initial analysis, the inventors determined specific dosage regimens predicted to provide equivalent exposure to that achievable with 300 mg Q4W IV. A dosage regimen of 105 mg subcutaneous weekly (QW) was initially found to provide an AUC ratio close to (or slightly greater than) 1 (FIG. 15A), even where projected bioavailability was reduced by ˜7% relative to that reported in Tummala et. al. 201826 (incorporated herein by reference in its entirety) to account for inter-individual variance in bioavailability (FIG. 15B). 105 mg subcutaneous QW appeared to provide comparable or improved median trough concentrations and IFNGS suppression as the comparative 300 Q4W mg IV dose (FIG. 16A and FIG. 16B). From these initial analyses it appeared that SC 105 mg QW dose of anifrolumab should be selected as equivalent to a 300 mg Q4W and thus as having the optimal efficacy/risk profile for the treatment of SLE patients. Importantly these analyses assumed that the 300 mg IV dose was close to the plateau of the dose response curve for anifrolumab.

### 10.2.2. Amended Selection of the Subcutaneous Dose for Anifrolumab

The inventors therefore first considered 105 mg QW to be the optimal SC dose of anifrolumab for the treatment of type I IFN mediated disease based on the data available from the MUSE study, Study 06 and Study 08. However, to confirm the selection of the 105 mg SC dose, the inventors conducted further analysis of the data from the TULIP I (Study 04) and TULIP II (Study 05) clinical trials.

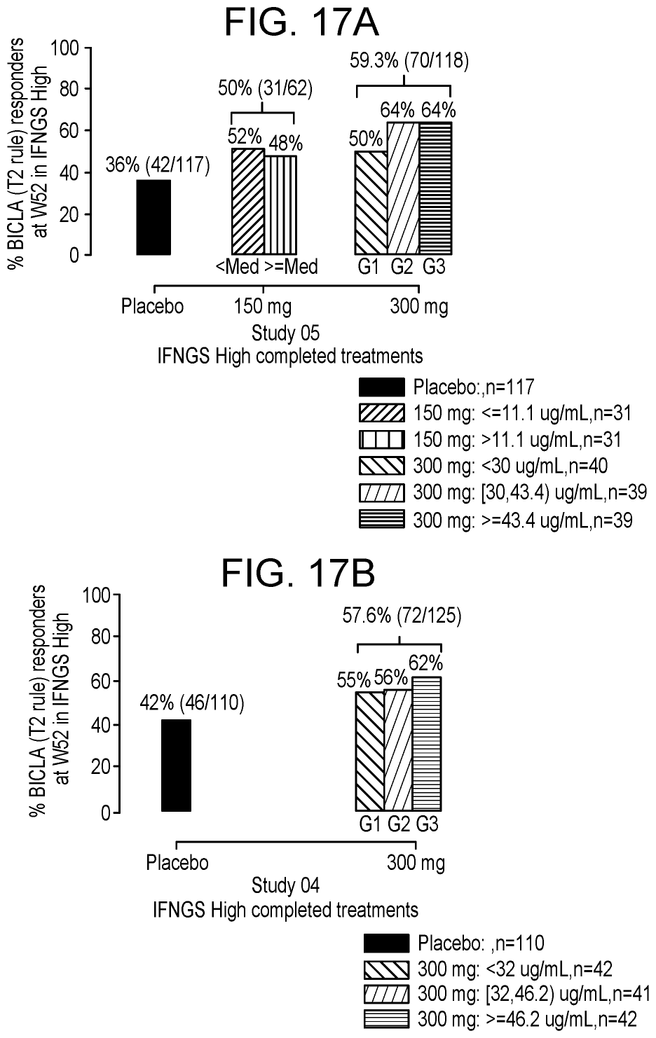

Using the additional data, a positive-exposure-BICLA relationship in IFNGS high patients was demonstrated. Surprisingly, this relationship was observed even within the 300 mg IV Q4W group (FIG. 17A and FIG. 17B). BICLA response within the 300 mg IV Q4W patient group was therefore variable. Logistic regression of the week 52 BILCA response in patients confirmed that PK exposure was a significant covariate in both TULIP I and TULIP II. Cₐᵥₑ was found to be statistically significant in both the analysis of all-corners and IFNGS high completed the treatments in both TULIP I and TULIP II independently and pooled TULIP I and TULIP II analysis. Exposure-response demonstrating higher Cₐᵥₑ were correlated with higher BICLA and SRI (4) in pooled data from the TULIP I and TULIP II studies. In other words, there was exposure-dependent variability in response to anifrolumab within SLE patients administered 300 mg Q4W IV (FIG. 17A and FIG. 17B).

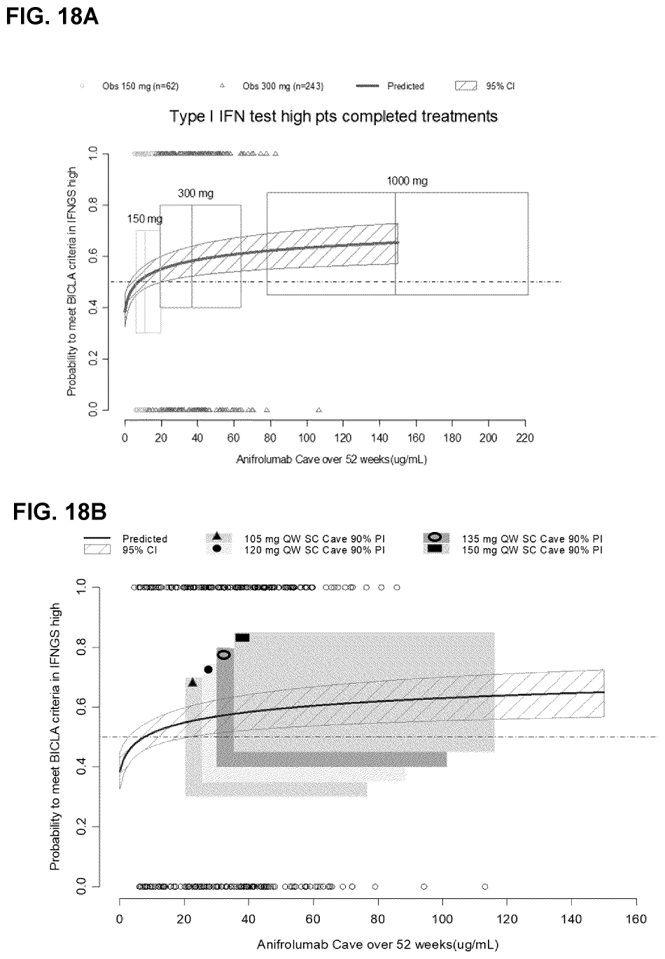

Surprisingly, the 300 mg IV Q4W dose was thus found to reside on the onset of the plateau of exposure response, whilst the suboptimal 150 mg IV dose resided in the step region of the exposure-response curve (FIG. 18A). As a consequence of these analyses, the inventors determined that a 105 mg QW subcutaneous dose (previously considered equivalent to a 300 mg IV Q4W dose) would not provide the optimal balance of efficacy and safety in SLE patients. The inventors thus determined to select another dose for SC administration that would mitigate the impact of variability in response a population of SLE patients.

In summary, from initial analysis, it appeared that administration of a subcutaneous dose of 105 mg QW anifrolumab would achieve at least a similar efficacy as 300 mg IV Q4W. However, surprisingly, following further analysis by the inventors of newly available data from further studies, it was found that the concentration of this weekly (QW) dose could be increased without reaching a maximum threshold in terms of bioavailability and efficacy. In other words, the QW dose could be increased beyond 105 mg to provide even greater blood plasma concentrations and IFNGS suppression, and to mitigate the observed variability in response in SLE patients. A dose of 105 mg would therefore be sub-optimal.

The surprising additional dose-response curve data were further validated by demonstrating that the probability of meeting a relevant BICLA response (in IFNGS high patients) was increased for weekly subcutaneous administration with concentrations higher than the 105 mg dose (Table 10-1: SC Efficacy Projection assuming no dose delays/interruptions.). These data demonstrate the unexpected position of the dose-response plateau (e.g. under subcutaneous administration), which shifts to the right for doses increasing above 105 mg (FIG. 18B), showing the maximal BICLA response is in fact achievable with a dose of greater than 105 mg and that a higher dose would be preferable (Table 10-1).

TABLE 10-1
SC Efficacy Projection assuming no dose delays/interruptions.

| | 90 mg | 105 mg | 120 mg | 135 mg | 150 mg |
|---|---|---|---|---|---|
| | SC QW | SC QW | SC QW | SC QW | SC QW |
| Equivalent IV dose | ~300 mg IV Q4W | | ~<400 mg | ~<450 mg | <500 mg |
| | | | IV Q4W | IV Q4W | IV Q4W |
| | | | | | ~300 mg |
| | | | | | SC Q2W |
| Median Cave ratio to 300 mg IV | 0.92 | 1.14 | 1.36 | 1.59 | 1.81 |
| % exceeded 95^{th} percentile | 3.3% | 9.4% | 20.1% | 33.5% | 48.9% |
| of 300 mg IV | | | | | |
| % overlapped with ≥5^{th} | 0.3% | 1.8% | 5% | 11% | 21% |
| percentile of 1000 mg IV | | | | | |
| % IFNGS high pts with 55% | ~86% | ~94% | ~98% | ~99% | ~100% |
| chance of BLCA response | | | | | |
| % IFNGS high pts with 60% | ~10% | ~23% | ~38% | ~55% | ~68% |
| chance of BLCA response | | | | | |

### 10.2.3. The Bioavailability of Anifrolumab is Highly Variable

Upon further investigation as to the bioavailability of anifrolumab, the inventors elucidated that a surprisingly high level of variability in anifrolumab bioavailability subsequent to subcutaneous administration may exist amongst different patients. The high level of variability in anifrolumab bioavailability was not appreciated in previous studies reporting >80% bioavailability for subcutaneous administration²⁶. The bioavailability (F1) of anifrolumab in Study 08 (SLE patients, SC) was found to be 81% in healthy volunteers using the population PK model (Table 10-2).

TABLE 10-2
Anifrolumab bioavailability based on healthy volunteers

| | Final IV SLE model | Study 06 HVs | |
|---|---|---|---|
| | (including 6 subjects | (300 mg IV: 6, 300 mg | |
| Parameters ± SE | from 06 IV arm) | SC: 6, 600 mg SC: 6) | |
| CL (IFNGS high) | 0.193 L/day | — | |
| CL (IFNGS low/HVs) | 0.153 L/day | 0.146 ± 0.036 L/day | |
| IIV: CL | 0.109 (CV: 33.1%) | 0.0431 | (CV: 20.8%) |
| Fa | — | 0.812 ± 0.12 | |
| Ka | — | 0.274 ± 0.124/day | |
| IIV: Ka | — | 0.221 | (CV: 47%) |

The bioavailability of a typical monoclonal antibody via subcutaneous injection ranged from 52-80%⁴⁹. The inventors conducted external validation of Study 08, Ph2 SC in SLE, using a PPK model developed with healthy volunteers and SLE patients from IV studies to determine the bioavailability in SLE population.

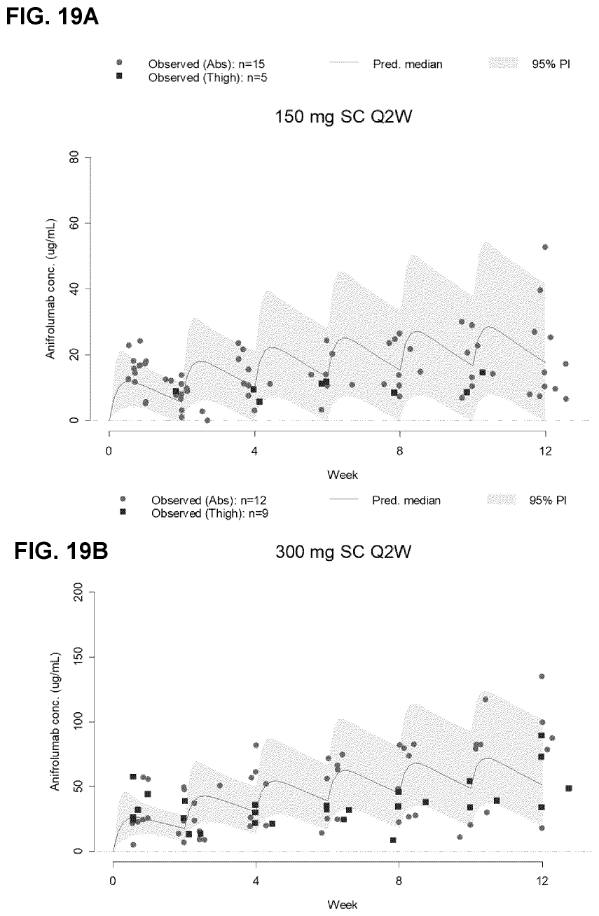

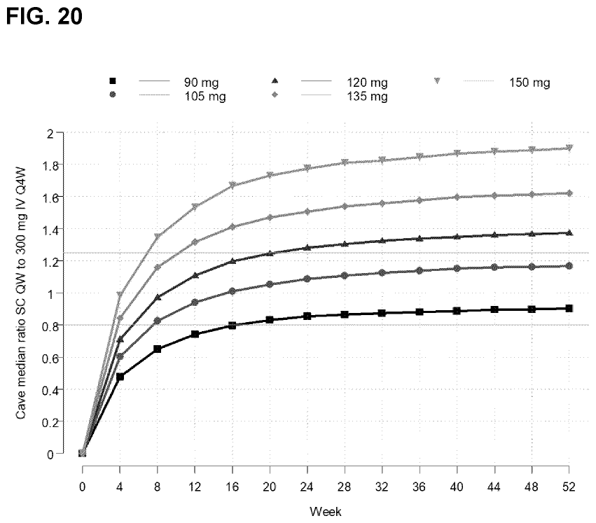

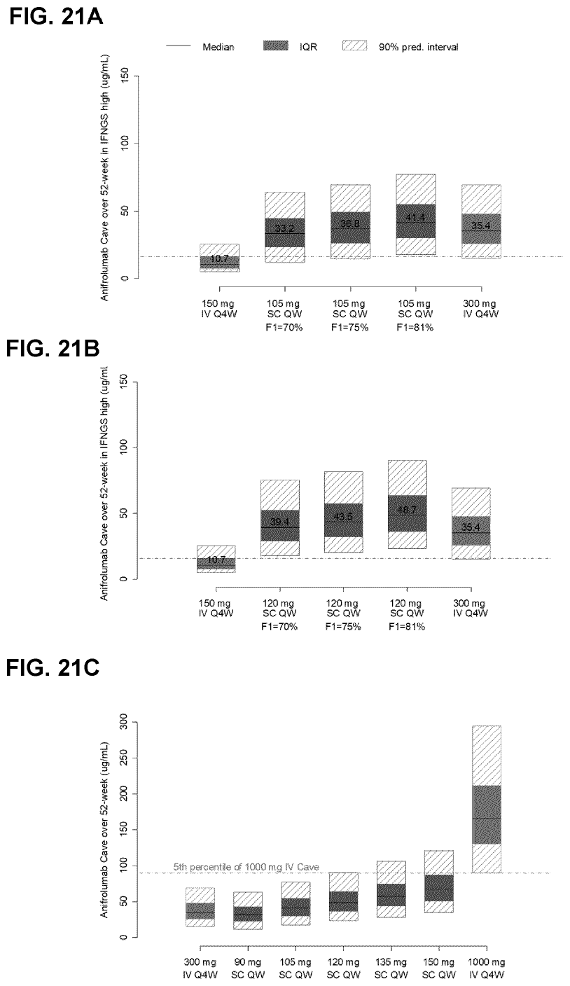

In-depth analysis of the data from Study 08 revealed that bioavailability was affected by SC administration site. In particular, when the bioavailability of 300 mg at the abdomen was estimated versus IV, the bioavailability (F1) was estimated to be 85.4% compared to 81% when the sites of injection was not taken into consideration. As such, C_{troughs} following injection at thigh trended downward compared to injection at abdomen (FIG. 19A and FIG. 19B). As such, it was surprisingly concluded that bioavailability may, in fact, be as low as 70%, taking into account variability due to injection site and the higher variability in bioavailability for SLE patients compared to healthy volunteers. Importantly, if a bioavailability (F1) of 81-87% was assumed, 105 mg was initially projected to provide a comparable Cₐᵥₑ to that of 300 mg IV (FIG. 20). By contrast, when the estimated bioavailability was reduced to ˜70% or less, the median Cₐᵥₑ of the 105 mg QW subcutaneous dose fell to below 1 (FIG. 21A, FIG. 21B and Table 10-3).

TABLE 10-3
Anifrolumab bioavailability

| | 90 mg | 105 mg | 120 mg | 135 mg | 150 mg |
|---|---|---|---|---|---|
| Bioavailability | SC QW | SC QW | SC QW | SC QW | SC QW |
| 82% | 0.92 | 1.14 | 1.36 | 1.59 | 1.81 |
| ~70% | 0.73 | 0.92 | 1.11 | 1.31 | 1.49 |
| ~60% | 0.57 | 0.73 | 0.89 | 1.06 | 1.22 |

Values = median Cₐᵥₑ to 300 mg IV;
SC = subcutaneous

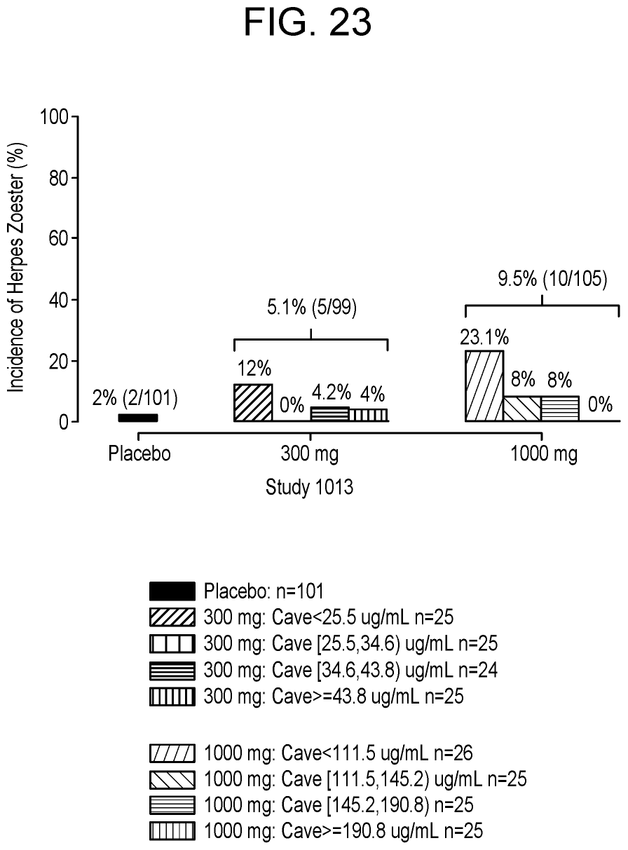

Furthermore, there was an undesirable 30% overlap in Cₐᵥₑ between 105 mg SC QW and the suboptimal IV dose, 150 mg Q4W versus the only 16% overlap observed when the bioavailability was assumed to be 81% (FIG. 21A). However, when a SC 120 mg dose was used, the Cₐᵥₑ overlap with the 150 mg IV dose was less than the overlap with the optimal IV dose of 300 mg IV, even when a low bioavailability of 70% was assumed (FIG. 21B). Furthermore, the 120 mg SC QW dose had minimal overlap with the undesirable 1000 mg IV dose (FIG. 21C), at which the risk of herpes zoster infection is increased (FIG. 23). A 150 mg SC QW dose had an undesirable overlap with the 1000 mg IV Q4W dose. Even more surprisingly, a SC dose of 120 mg or more was projected to have better PD suppression (Table 10-4) than the assumed optimal 300 mg IV dose (Table 10-5).

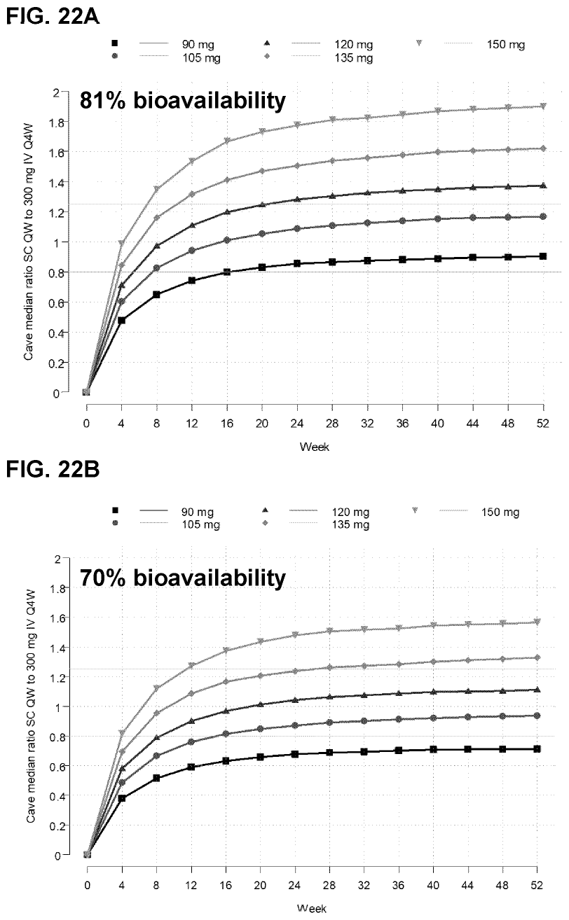

Selection of a dose higher than 105 mg, preferably 120 mg or higher, therefore optimizes the exposure-response by minimizing the impact of the variability of the onset of response and bioavailability in patients with SLE (Table 10-4, FIG. 22A and FIG. 22B). A SC dose of below 150 mg QW is also desirable to reduce the risk of herpes zoster infection.

TABLE 10-4
Calculated % PD suppression at week 24, SC dose

| SC | WK 24 Suppression (%) | | |
|---|---|---|---|
| Dose (mg) | 75% | 80% | 90% |
| 90 | 89.0 | 84.6 | 63.8 |
| 105 | 92.9 | 89.8 | 69.2 |
| 120 | 94.8 | 91.9 | 74.2 |
| 135 | 96.0 | 93.9 | 75.8 |
| 150 | 96.5 | 94.6 | 80.2 |

TABLE 10-5
Calculated % PD suppression at week 24, IV dose

| IV | WK 24 Suppression (%) | | |
|---|---|---|---|
| Dose (mg) | 75% | 80% | 90% |
| 300 | 74.2 | 68.3 | 42.5 |
| 400 | 82.9 | 77.9 | 54.7 |
| 450 | 85.9 | 80.8 | 56.4 |
| 500 | 88.7 | 84.8 | 62.5 |
| 600 | 92.7 | 88.8 | 68.9 |
| 1000 | 96.9 | 94.5 | 80.2 |

Doses of 120 mg and 135 mg QW particularly provide reasonable benefit-risk profiles. At doses at 150 mg QW or above, there is an increase in safety risk e.g. an increase in the risk of herpes zoster in patients, given that a SC dose of 150 mg QW is equivalent to a 1000 mg IV Q4W (FIG. 21C, FIG. 23). A subcutaneous dose of less than 150 mg QW and more than 105 mg QW was therefore determined as the preferred dose. A subcutaneous dose of less than 150 mg QW and less or equal to 135 mg was determined as the more preferred dose. A subcutaneous dose of 120 mg was determined as optimal dose.

To summarize, the inventors have surprisingly found that the optimal subcutaneous dose of anifrolumab may first appear to be 105 mg QW given the preliminary data that was previously available (FIG. 12). However, further data and analyses surprisingly revealed that a dose of 105 mg QW or lower would under-dose a significant proportion of patients (FIG. 18B, Table 10-3). Thus, a particularly advantageous dosing regimen demonstrated by the inventors was doses higher than 105 mg QW. A particularly optimal dose was determined to be 120 mg subcutaneous QW, which is equivalent to approximately 400 mg IV Q4W, depending on estimated bioavailability. The optimal SC dose is therefore surprisingly >30% higher than what would be considered optimal based solely on a comparison with 300 mg IV Q4W and the previously understood bioavailability of anifrolumab.

The inventors have thus surprisingly demonstrated that a dose of greater than 105 mg SC QW and less than 150 mg SC QW, and in particular a dose of 120 mg QW (a) maximizes efficacy whilst maintaining an acceptable safety profile, (b) mitigates the impact of variability in bioavailability and (c) mitigates the impact of variability in the onset of response. Thus, dosing at greater than 105 mg QW advantageously accounts for the variance in bioavailability, leading to improved therapeutic outcome. A dose of less than 150 mg QW mitigates the risk of herpes zoster infection.

Pharmacokinetic data in healthy volunteers (study 06 [IV arm only]) and in patients with SLE (Studies 1013, 02, 04, and 05) were also pooled to evaluate the impacts of covariates, such as demographics and renal/liver function tests, on PK exposure. Higher body weight and type I IFN test high patients were found to have significantly higher clearance (CL) and lower concentrations. However, surprisingly there was no clinically relevant impact of these covariates on efficacy and safety. Surprisingly, other covariates pertaining to specific populations evaluated in population PK modeling were not found to be significant including race/ethnicity/region, age, gender, renal/hepatic function tests, standard of care therapy (e.g., OCS, anti-malarial, azathioprine, methotrexate, mycophenolate mofetil, mycophenolic acid, mizoribine, and NSAIDs), and commonly used medications in SLE patients (ACE inhibitors and HMG-COA reductase inhibitors).

### 10.3. Conclusion

The present inventors have demonstrated that an anifrolumab dose of <150 mg Q and >105 mg QW will provide at least similar or even a higher Cₐᵥₑ over 52 weeks to that of 300 mg IV Q4W. A 120 mg SC QW dose will particularly provide an efficacy at least equivalent to that demonstrated for a 300 mg IV Q4W dose. It is further plausibly demonstrated that a 120 mg SC QW dose will provide an efficacy greater than that demonstrated for a 300 mg IV Q4W dose.

On the basis of the data demonstrated herein a subcutaneous dose of anifrolumab for has been selected for a multi-center, randomized, double-blind, placebo-controlled, phase 3 study evaluating the efficacy and safety of subcutaneous anifrolumab in adult patients with SLE. The study design is shown in FIG. 27A. In summary, two doses of SC anifrolumab (150 mg and 300 mg every 2 weeks [Q2W]) were evaluated in the completed Phase 2 SC study in SLE patients with Type I IFN test high results and active skin disease (Study 06). The primary pharmacokinetic (PK)/pharmacodynamic (PD) endpoints of the Phase 2 SC study were analyzed at Week 12 and safety, and tolerability of SC administration of anifrolumab was assessed through Week 52. Based on PK/PD data from the Phase 2 SC study as well as data from anifrolumab IV studies, a dose of 120 mg QW was selected for this current Phase 3 SC study to provide comparable and noninferior average concentration (Cₐᵥₑ) to 300 mg IV in a single injection and hence 120 mg SC QW is expected to provide at least similar efficacy to 300 mg IV Q4W.

Given the change of dosing interval from Q4W to QW and by providing at least similar Cₐᵥₑ, the troughs concentrations of 120 mg SC QW are projected to be higher than those of 300 mg IV Q4W, and hence it is expected to provide noninferior PD suppression to that of 300 mg IV. In addition, the Cₐᵥₑ of 120 mg SC QW over 52 weeks has minimal overlap with that of 1000 mg IV (evaluated in the Phase 2b Study 1013) which was shown to be safe and tolerable, and thus, any dose equivalent to below 1000 mg IV Q4W is considered to be safe.

Development of a SC route of administration using APFS of Al for anifrolumab is expected to provide increased convenience and dosing flexibility and reduced exposure to infection risk related to clinic visits for dosing (including but not limited to influenza or COVID-19) for patients and/or caregivers and to improve treatment accessibility and compliance.

### 11. Example 5: Classification of Patients With Systemic Lupus Erythematosus Enrolled in 2 Phase 3 Trials Using EULAR/ACR 2019 Criteria

### 11.1. Background/Purpose

The TULIP-1 and TULIP-2 trials of anifrolumab, an anti-type I IFN receptor antibody, enrolled autoantibody-positive (ANA, anti-dsDNA, or anti-Smith [anti-Sm]) patients, who fulfilled the ACR 1997 classification criteria for SLE14.16.24. The aim of this analysis was to assess how many patients who participated in the TULIP trials also met the updated EULAR/ACR 2019 criteria²⁵.

### 11.2. Methods

TULIP-1 (NCT02446912) and TULIP-2 (NCT02446899) were randomized, placebo-controlled, 52-week trials of intravenously administered anifrolumab in patients with moderate to severe SLE despite standard therapy. Inclusion criteria included fulfilling at least 4 of the ACR 1997 criteria for SLE, positive ANA and/or anti-dsDNA and anti-Sm antibodies, and moderate to severe SLE. Data for investigating classification using EULAR/ACR 2019 criteria were combined from the ACR criteria, BILAG-specific SLE history, and documented medical history.

### 11.3. Results

The TULIP-1 and TULIP-2 data pool included 726 patients with SLE. Of these, all but 2 (99.7%) met the ACR 1997 SLE criteria (Table). The EULAR/ACR 2019 classification criteria for SLE were met by 712/726 patients (98.1%). Thus, most patients (97.9% [711/726]) were concordant in meeting both the ACR 1997 and EULAR/ACR 2019 SLE classification criteria. Of the patients classified as having SLE using ACR 1997 criteria, 1.8% (13/726) did not meet the EULAR/ACR 2019 criteria. Of these 13 discordant patients, 8 were ANA negative but either anti-dsDNA or anti-Sm antibody positive. Two patients did not meet ACR 1997 criteria at baseline; one patient was not classified as having SLE using either ACR 1997 or EULAR/ACR 2019 criteria, and the other patient was classified as having SLE using EULAR/ACR 2019 criteria. This latter discordant patient did not meet ACR 1997 criteria, having just nonerosive arthritis and positive ANA, but met EULAR/ACR 2019 criteria with positive ANA, fever, nonscarring alopecia, and joint involvement. At study baseline, positive ANA (97.2%) and nonerosive arthritis (97.5%) were the 2 most frequent ACR 1997 criteria among all patients.

TABLE 11-1
Classification According to ACR 1997 or EULAR/ACR 2019
in Patients Enrolled in the TULIP-1 and TULIP-2 Trials

| | | | |
|---|---|---|---|
| | | Anifrolumab | |
| | Placebo | 300 mg | Total |
| Patients, n (%) | (n = 366) | (n = 360) | (n = 726) |

ACR 1997 classification

| | | | |
|---|---|---|---|
| Patients Classified SLE | 364 | 360 | 724 |
| | (99.5) | (100) | (99.7) |
| Patients not classified SLE | 2 (0.5) | 0 | 2 (0.3) |

EULAR/ACR 2019 classification

| | | | |
|---|---|---|---|
| Patients classified SLE | 361 | 351 | 712 |
| | (98.6) | (97.5) | (98.1) |
| Patients not classified SLE | 5 (1.4) | 9 (2.5) | 14 (1.9) |
| Concordance | 361 | 351 | 712 |
| | (98.6) | (97.5) | (98.1) |
| Patients classified SLE according | 360 | 351 | 711 |
| to both classifications | (98.4) | (97.5) | (97.9) |
| Patients not classified SLE | 1 (0.3) | 0 | 1 (0.1) |
| according to both classifications | | | |
| Discordance | 5 (1.4) | 9 (2.5) | 14 (1.9) |
| Not SLE using ACR 1997 but | 1 (0.3) | 0 | 1 (0.1) |
| SLE using EULAR/ACR 2019 | | | |
| SLE using ACR 1997 but not | 4 (1.1) | 9 (2.5) | 13 (1.8) |
| SLE using EULAR/ACR 2019 | | | |

### 11.4. Conclusions

Nearly all patients enrolled in the TULIP-1 and TULIP-2 trials were classifiable as having SLE using both the ACR 1997 classification criteria and the EULAR/ACR 2019 criteria. Of the patients not meeting the new criteria, most were ANA negative but had detectable autoantibodies against dsDNA and/or Sm.

### 12. Example 6: Treatment of Refractory SLE

### 12.1. Background

In 2 phase 3 trials, TULIP-1 and TULIP-2, anifrolumab, a type I IFN receptor monoclonal antibody, improved disease activity in patients with SLE. Here, we compared the efficacy of anifrolumab in patients with recent onset vs established SLE disease (defined by time since diagnosis), using pooled data from the TULIP trials.

### 12.2. Methods

TULIP-1 (NCT02446912) and TULIP-2 (NCT02446899) were randomized, placebo-controlled, 52-week trials of intravenous anifrolumab 300 mg every 4 weeks for 48 weeks in patients with moderate to severe SLE despite standard therapy.1,2 All patients were aged 18-70 years and met the ACR criteria for SLE. Baseline characteristics and BILAG-based Composite Lupus Assessment (BICLA) response rates at Week 524 for anifrolumab 300 mg vs placebo were compared between patients who at the time of their baseline study visits were within 2 years of their SLE diagnosis (recent onset) and patients who were diagnosed beyond 2 years (established). Efficacy was analyzed with a stratified Cochran-Mantel-Haenszel approach controlling for randomization stratifications factors and study.

### 12.3. Results

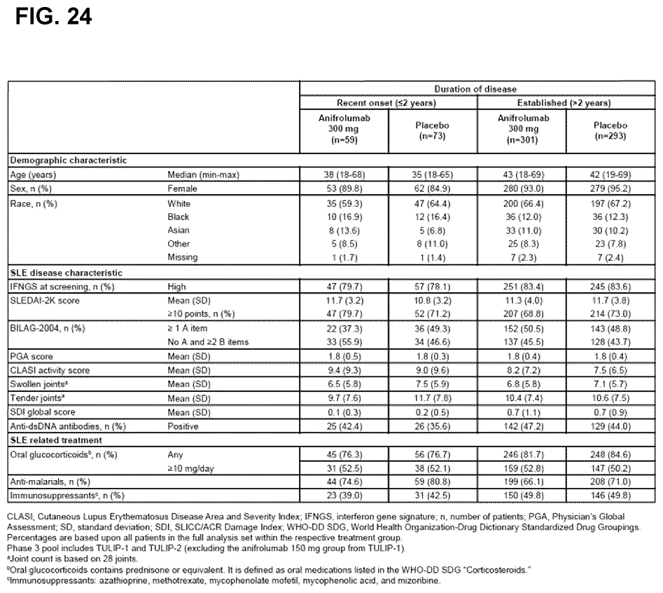

Of the 726 patients included from TULIP-1 and TULIP-2 (anifrolumab, n=360; placebo, n=366), 594 had established disease (anifrolumab, n=301; placebo, n=293), and 132 had recent onset disease (anifrolumab, n=59; placebo, n=73) at baseline. In contrast to patients with recent onset disease, patients with established disease had a higher median age (43 vs 37 years) and were more likely to be female (94.1% vs 87.1%), IFN gene signature high (83.5% vs 78.8%), anti-dsDNA positive (45.6% vs 38.6%), have ≥1 BILAG-2004 A item (49.7% vs 43.9%), and be receiving oral glucocorticoids (83.2% vs 76.5%) or immunosuppressants (49.8% vs 40.9%) at baseline (FIG. 24). Patients with established vs recent-onset disease were more likely to be IFN gene signature (IFNGS) high (83.5% vs 78.8%), anti-dsDNA antibody-positive (45.6% vs 38.6%), complement 4 (C4) low (24.7% vs 16.7%), have ≥1 BILAG-2004 A item (49.7% vs 43.9%), and have a higher mean global SDI score (0.7 vs 0.1). Patients with established vs recent-onset disease were more likely to be receiving oral glucocorticoids (GC) (83.2% vs 76.5%), and/or immunosuppressants (49.8% vs 40.9%), but not antimalarials (69.5% vs 78.0%).

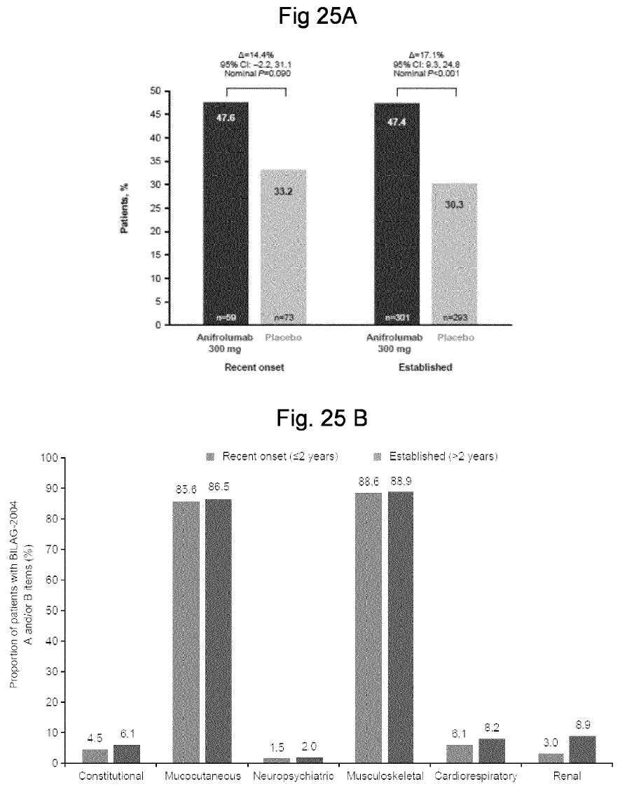

The numbers of BILAG-2004 A or B items across organ domains were comparable in patients with established or recent onset disease, excluding the renal domain, where a higher proportion of patients with established disease had more severe scores (A or B items; 8.9% vs 3.0%) (FIG. 25).

Treatment benefit of anifrolumab vs placebo, assessed by BICLA responses at Week 52, occurred in patients with established (difference [95% CI] 17.1% [9.3, 24.8], nominal P<0.001) and recent onset disease (difference [95% confidence intervals (CI)] 14.4% [-2.2, 31.1], nominal P=0.090).

### 12.4. Conclusions

Data from the TULIP trials provide evidence for the efficacy of anifrolumab in patients with SLE who have either established or recent onset disease. Patients with established SLE disease were more likely to be IFNGS high, receiving immunosuppressants at baseline, anti-dsDNA antibody-positive, and were more likely to have low C4 levels, renal involvement, and higher global organ damage scores compared with patients with recent-onset disease, possibly indicating more severe and/or refractory disease. Despite differences in baseline characteristics between patients with established and recent-onset disease, BICLA response rates with anifrolumab were similar in patients with established vs recent-onset disease. Data from the TULIP trials support the efficacy of anifrolumab in patients with SLE with severe, refractory and/or established disease.

### 13. Example 7: SLE Treatment History and Anifrolumab Efficacy by Baseline Standard Therapies in Patients with Systemic Lupus Erythematosus from 2 Phase 3 Trials

### 13.1. Background

In the phase 3 TULIP-1 and TULIP-2 trials, anifrolumab, a type I IFN receptor monoclonal antibody, improved disease activity versus placebo in patients who had moderate to severe SLE despite standard therapy with oral glucocorticoids (GCs), antimalarials, and/or immunosuppressants (refractory disease). The inventors investigated prior standard therapy use, and whether baseline standard therapy impacted anifrolumab efficacy in pooled data from TULIP-1 and TULIP-2.

### 13.2. Methods

TULIP-1 (NCT02446912) and TULIP-2 (NCT02446899) were 52-week trials of intravenous anifrolumab 300 mg or placebo every 4 weeks for 48 weeks, in which eligible patients fulfilled the ACR criteria for SLE. At screening, all patients had moderate to severe SLE (SLEDAI-2K ≥6, ≥1 A or ≥2 B BILAG-2004 organ domain scores, Physician's Global Assessment >1) and were required to be receiving ≥1 of the following: oral GCs, antimalarials, immunosuppressants (azathioprine, mizoribine, mycophenolate mofetil, mycophenolic acid, and/or methotrexate). Patients were divided into subgroups of SLE treatments at baseline. British Isles Lupus Assessment Group-based Combined Lupus Assessment (BICLA) response at Week 52 was compared across baseline SLE treatment subgroups using a stratified Cochran-Mantel-Haenszel approach.

### 13.3. Results

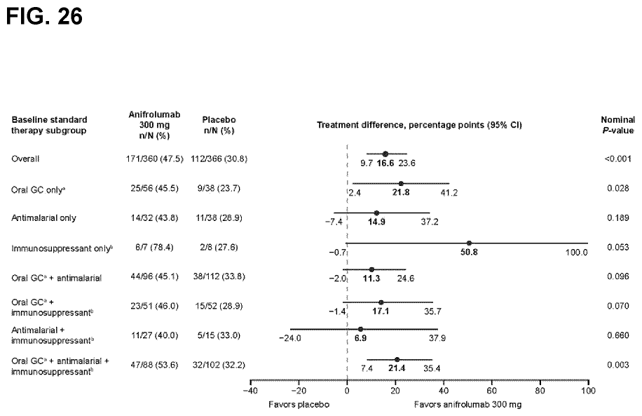

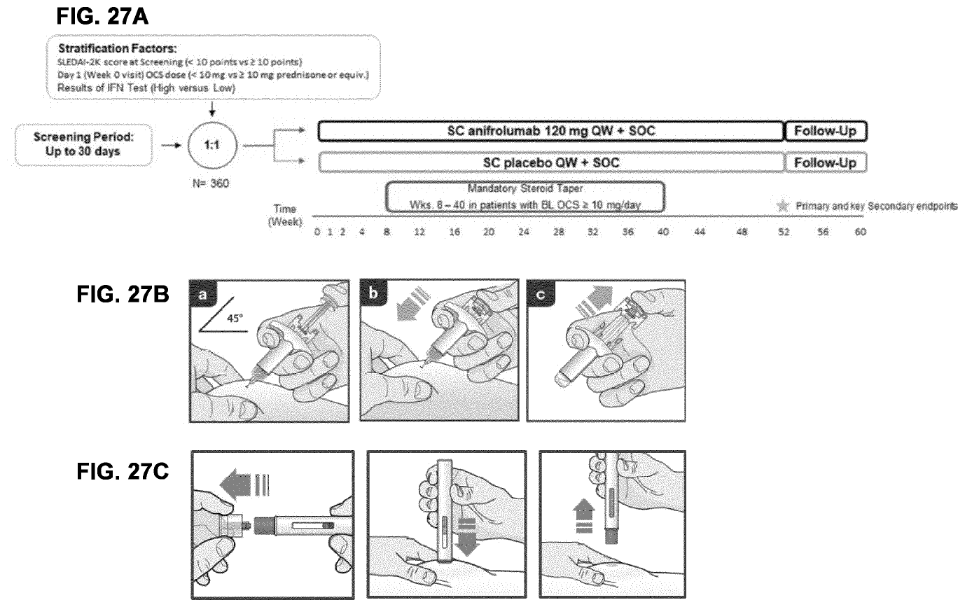

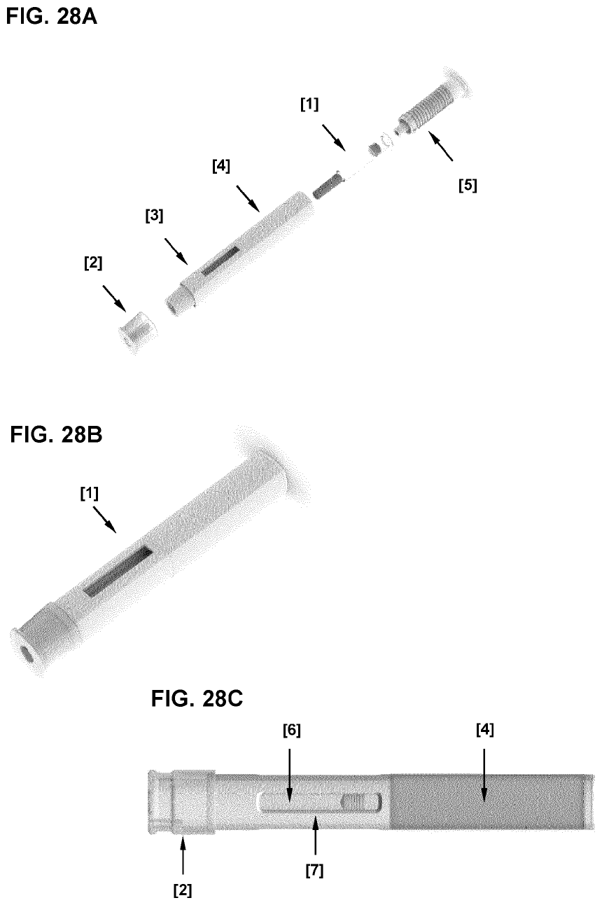

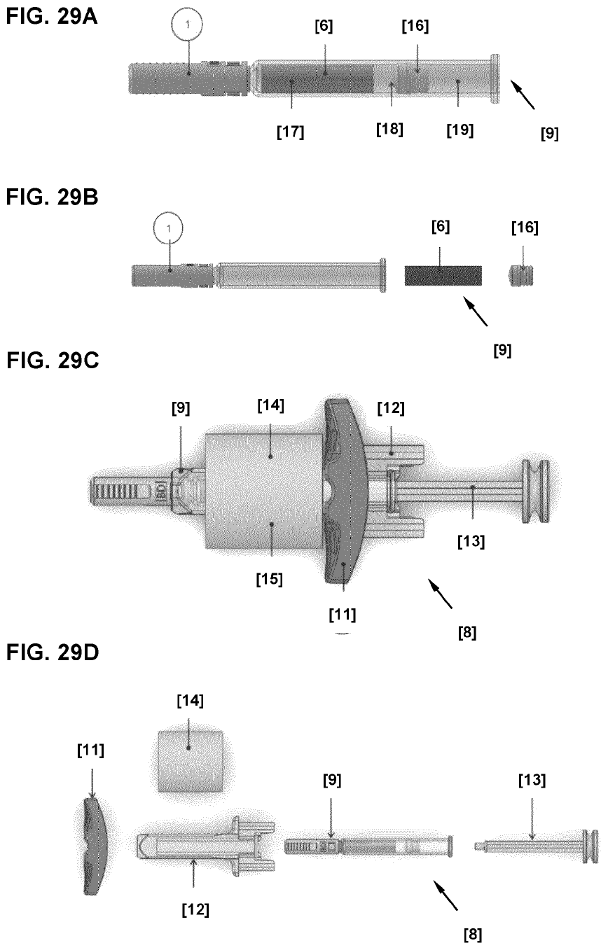

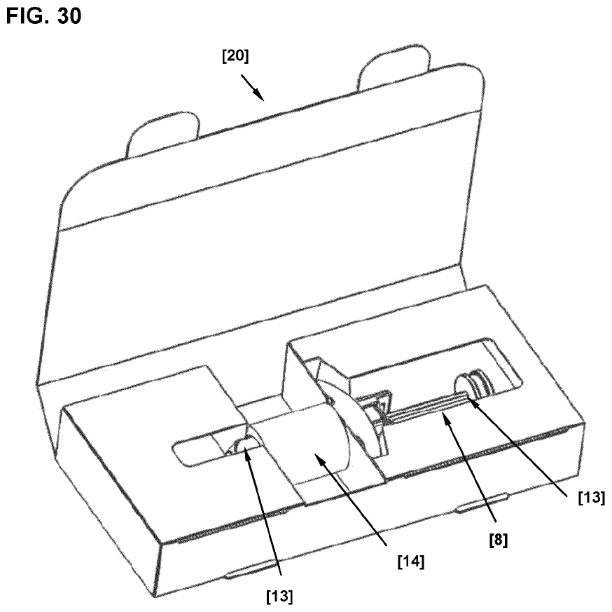

Overall, 726 patients received anifrolumab 300 mg (n=360) or placebo (n=366) in TULIP-1 and TULIP-2. Demographics and baseline disease characteristics were generally balanced between treatment groups. The median time from SLE diagnosis to randomization (prior to baseline) was 84.5 months, during which 89.5% of patients had received GCs, 84.3% had received antimalarials, and 68.0% had received immunosuppressants. Prior to baseline, all patients had received ≥1 SLE-related therapy, 34.3% had received 2 SLE-related therapies, and 57.3% of patients had received ≥3 SLE-related therapies. At baseline, patients were receiving GCs (82.0%), antimalarials (70.2%), and/or immunosuppressants (48.2%), with most patients receiving combinations of the three (Table 13-1). Anifrolumab 300 mg was associated with higher BICLA response rates versus placebo across all evaluated baseline SLE standard therapy subgroups, with positive treatment differences ranging from 6.9% (antimalarial+immunosuppressant) to 50.8% (immunosuppressant only) (FIG. 26); however, some groups had small sample sizes and the impact of dosage on efficacy was not investigated. Furthermore, positive treatment differences favoring anifrolumab 300 mg vs placebo were observed in patients who were receiving GCs+antimalarials+immunosuppressants at baseline (53.6% vs 32.2%; Δ=21.4%; 95% CI: 7.4-35.4), who were likely to have treatment-refractory disease.

TABLE 13-1
Background standard therapy regimens prior
to and at baseline in TULIP-1 and TULIP-2

| | Prior to baseline^{a} | | At baseline^{b} | |
|---|---|---|---|---|
| | Anifrolumab | | Anifrolumab | |
| | 300 mg | Placebo | 300 mg | Placebo |
| SLE standard therapies | (n = 360) | (n = 366) | (n = 360) | (n = 366) |
| Any oral GC^{c} | 325 (90.3) | 325 (88.8) | 291 (80.8) | 304 (83.1) |
| Oral GC only | 28 (7.8) | 21 (5.7) | 56 (15.6) | 38 (10.4) |
| Oral GC + antimalarial and/or | 297 (82.5) | 304 (83.1) | 235 (65.3) | 266 (72.7) |
| immunosuppressant | | | | |
| Any antimalarials | 299 (83.1) | 313 (85.5) | 243 (67.5) | 267 (73.0) |
| Antimalarial only | 28 (7.8) | 27 (7.4) | 32 (8.9) | 38 (10.4) |
| Antimalarial + oral GC and/or | 271 (75.3) | 286 (78.1) | 211 (58.6) | 229 (62.6) |
| immunosuppressant | | | | |
| Any immunosupressants | 248 (68.9) | 246 (67.2) | 173 (48.1) | 177 (48.4) |
| Azathioprine | 121 (33.6) | 113 (30.9) | 62 (17.2) | 61 (16.7) |
| Cyclophosphamide | 50 (13.9) | 39 (10.7) | — | — |
| Leflunomide | 9 (2.5) | 9 (2.5) | — | — |
| Methotrexate | 104 (28.9) | 135 (36.9) | 56 (15.6) | 73 (19.9) |
| Mizoribine | 9 (2.5) | 11 (3.0) | 4 (1.1) | 3 (0.8) |
| Mycophenolate^{d} | 82 (22.8) | 79 (21.6) | 54 (15.0) | 45 (12.3) |
| Tacrolimus | 18 (5.0) | 23 (6.3) | — | — |
| ≥2 different immunosupressants | 103 (28.6) | 102 (27.9) | 3 (0.8) | 5 (1.4) |

GC, glucocorticoid
^{a}Includes any SLE standard therapies used since SLE diagnosis with start date prior to randomization;
^{b}Baseline is defined as the last measurement prior to randomization and investigational product dose administration Day 1;
^{c}Prednisone or equivalent;
^{d}Mycophenolate or mycophenolic acid.

### 13.4. Conclusions

In 2 phase 3 trials, there were consistently higher BICLA response rates with anifrolumab 300 mg than with placebo, regardless of SLE standard therapy usage, including in patients with potentially more treatment-refractory SLE that required treatment with GCs, immunosuppressants, and antimalarials. Pooled data from 2 phase 3 trials showed that the treatment effect with anifrolumab 300 mg for BICLA response was consistent, regardless of baseline standard therapy usage. In particular, the consistent treatment response for the subgroup of patients with the greatest use of standard therapies at baseline plausibly demonstrates that anifrolumab treatment provides benefit to patients with refractory disease

### 14. Example 8: Injection Device

Anifrolumab is administered by an injection device [1] [9] such as a prefilled syringe (PFS) (FIG. 27A) or an autoinjector (AI) (FIG. 27B).

### 14.1. Autoinjector

Anifrolumab may be administered by an autoinjector [1]. The autoinjector is shown in exploded view (FIG. 28A) and in an assembled form (FIG. 28B). A label [4] is wrapped around and attached to the autoinjector [1] (FIG. 28C). The autoinjector has an autoinjector housing [3], cap and cap remover [2] and drive unit [5]. The liquid anifrolumab formulation unit dose [6] is contained in the autoinjector housing [3]. The unit dose [6] can be viewed through the viewing window [7].

### 14.2. Accessorized Pre-Frilled Syringe

Anifrolumab may be administered by accessorized pre-filled syringe (APFS) [8]. The APFS [8] includes the unit dose of anifrolumab [6] contained in a primary container [9] shown in an assembled state in FIG. 29A and in an exploded view in FIG. 29B. The primary container [9] has a plunger stopper [16]. The primary container has a nominal fill volume of 0.8 ml but may contain slightly more than 0.8 ml. The remainder of the space in the primary container [9] is taken up by an air bubble [18]. The air bubble may have a size of 3-5 mm, optionally, 4 mm. The primary container [9] has a defined stopper position [19].

The accessorized pre-filled syringe (APFS) primary container [9] is provided in a PFS assembly [8] including a needle guard [12], a finger flange and a plunger rod [13]. A label is provided with the primary container [9] in the PFS assembly [8]. The label is wrapped around the syringe [9] in the label placement position [15].

### 14.3. Packaging

The injection device [1] [8] is provided in a kit (FIG. 30). A label [4] is provided with the APFS or autoinjector in the packaging. The label includes instruction for the use of the injection device [1], [8]. The packaging includes a tamper seal.

### REFERENCES

All publications mentioned in the specification are herein incorporated by reference.
(1) Pons-Estel, G. J.; Alarcón, G. S.; Scofield, L.; Reinlib, L.; Cooper, G. S. Understanding the Epidemiology and Progression of Systemic Lupus Erythematosus. Semin. Arthritis Rheum. 2010, 39 (4), 257-268. https://doi.org/10.1016/j.semarthrit.2008.10.007.
(2) McCauliffe, D. P. Cutaneous Lupus Erythematosus. Semin. Cutan. Med. Surg. 2001, 20 (1), 14-26. https://doi.org/10.1053/sder.2001.23091.
(3) Uva, L.; Miguel, D.; Pinheiro, C.; Freitas, J. P.; Marques Gomes, M.; Filipe, P. Cutaneous Manifestations of Systemic Lupus Erythematosus. Autoimmune Dis. 2012, 2012, 834291. https://doi.org/10. 1155/2012/834291.
(4) Zayat, A. S.; Md Yusof, M. Y.; Wakefield, R. J.; Conaghan, P. G.; Emery, P.; Vital, E. M. The Role of Ultrasound in Assessing Musculoskeletal Symptoms of Systemic Lupus Erythematosus: A Systematic Literature Review. Rheumatol. Oxf. Engl. 2016, 55 (3), 485-494. https://doi.org/10.1093/rheumatology/kev343.
(5) Sa, C.; E, A.; A, R.; D, I. Damage and mortality in a group of British patients with systemic lupus erythematosus followed up for over 10 years https://pubmed.ncbi.nlm.nih.gov/19359343/(accessed 2021 Feb. 8). https://doi.org/10.1093/rheumatology/kep062.
(6) Murimi-Worstell, I. B.; Lin, D. H.; Nab, H.; Kan, H. J.; Onasanya, O.; Tierce, J. C.; Wang, X.; Desta, B.; Alexander, G. C.; Hammond, E. R. Association between Organ Damage and Mortality in Systemic Lupus Erythematosus: A Systematic Review and Meta-Analysis. BMJ Open 2020, 10 (5), e031850. https://doi.org/10.1136/bmjopen-2019-031850.
(7) Doria, A.; Briani, C. Lupus: Improving Long-Term Prognosis. Lupus 2008, 17 (3), 166-170. https://doi.org/10.1177/0961203307087612.
(8) Petri, M. Long-Term Outcomes in Lupus. Am. J. Manag. Care 2001, 7 (16 Suppl), S480-485.
(9) Zonana-Nacach, A.; Barr, S. G.; Magder, L. S.; Petri, M. Damage in Systemic Lupus Erythematosus and Its Association with Corticosteroids. Arthritis Rheum. 2000, 43 (8), 1801-1808. https://doi.org/10.1002/1529-0131 (200008) 43: 8<1801::AID-ANR16>3.0.CO; 2-O.
(10) Urowitz, M. B.; Bookman, A. A.; Koehler, B. E.; Gordon, D. A.; Smythe, H. A.; Ogryzlo, M. A. The Bimodal Mortality Pattern of Systemic Lupus Erythematosus. Am. J. Med. 1976, 60 (2), 221-225. https://doi.org/10.1016/0002-9343 (76) 90431-9.
(11) Patel, D. D.; Antoni, C.; Freedman, S. J.; Levesque, M. C.; Sundy, J. S. Phase 2 to Phase 3 Clinical Trial Transitions: Reasons for Success and Failure in Immunologic Diseases. J. Allergy Clin. Immunol. 2017, 140 (3), 685-687. https://doi.org/10.1016/j.jaci.2017.04.029.
(12) Dowden, H.; Munro, J. Trends in Clinical Success Rates and Therapeutic Focus. Nat. Rev. Drug Discov. 2019, 18 (7), 495-496. https://doi.org/10.1038/d41573-019-00074-z.
(13) Eisenberg, R. WHY CAN'T WE FIND A NEW TREATMENT FOR SLE? J. Autoimmun. 2009, 32 (3-4), 223-230. https://doi.org/10.1016/j.jaut.2009.02.006.
(14) Morand, E. F.; Furie, R.; Tanaka, Y.; Bruce, I. N.; Askanase, A. D.; Richez, C.; Bae, S.-C.; Brohawn, P. Z.; Pineda, L.; Berglind, A.; Tummala, R. Trial of Anifrolumab in Active Systemic Lupus Erythematosus. N. Engl. J. Med. 2020, 382 (3), 211-221. https://doi.org/10.1056/NEJMoa1912196.
(15) Furie, R.; Khamashta, M.; Merrill, J. T.; Werth, V. P.; Kalunian, K.; Brohawn, P.; Illei, G. G.; Drappa, J.; Wang, L.; Yoo, S.; Investigators, for the C. S. Anifrolumab, an Anti-Interferon a Receptor Monoclonal Antibody, in Moderate to Severe Systemic Lupus Erythematosus. Arthritis Rheumatol. Hoboken Nj 2017, 69 (2), 376. https://doi.org/10.1002/art.39962.
(16) Furie, R. A.; Morand, E. F.; Bruce, I. N.; Manzi, S.; Kalunian, K. C.; Vital, E. M.; Ford, T. L.; Gupta, R.; Hiepe, F.; Santiago, M.; Brohawn, P. Z.; Berglind, A.; Tummala, R. Type I Interferon Inhibitor Anifrolumab in Active Systemic Lupus Erythematosus (TULIP-1): A Randomised, Controlled, Phase 3 Trial. Lancet Rheumatol. 2019, 1 (4), e208-e219. https://doi.org/10.1016/S2665-9913 (19) 30076-1.
(17) Isenberg, D. A.; Merrill, J. T. Why, Why, Why de-Lupus (Does so Badly in Clinical Trials). Expert Rev. Clin. Immunol. 2016, 12 (2), 95-98. https://doi.org/10.1586/1744666X.2016.1112270.
(18) Wallace, D. J. The Evolution of Drug Discovery in Systemic Lupus Erythematosus. Nat. Rev. Rheumatol. 2015, 11 (10), 616-620. https://doi.org/10.1038/nrrheum.2015.86.
(19) Felten, R.; Sagez, F.; Gavand, P.-E.; Martin, T.; Korganow, A.-S.; Sordet, C.; Javier, R.-M.; Soulas-Sprauel, P.; Rivière, M.; Scher, F.; Poindron, V.; Guffroy, A.; Arnaud, L. 10 Most Important Contemporary Challenges in the Management of SLE. Lupus Sci. Med. 2019, 6 (1), e000303. https://doi.org/10.1136/lupus-2018-000303.
(20) Gladman, D.; Ginzler, E.; Goldsmith, C.; Fortin, P.; Liang, M.; Urowitz, M.; Bacon, P.; Bombardieri, S.; Hanly, J.; Hay, E.; Isenberg, D.; Jones, J.; Kalunian, K.; Maddison, P.; Nived, O.; Petri, M.; Richter, M.; Sanchez-Guerrero, J.; Snaith, M.; Sturfelt, G.; Symmons, D.; Zoma, A. The Development and Initial Validation of the Systemic Lupus International Collaborating Clinics/American College of Rheumatology Damage Index for Systemic Lupus Erythematosus. Arthritis Rheum. 1996, 39 (3), 363-369. https://doi.org/10. 1002/art. 1780390303.
(21) Albrecht, J.; Taylor, L.; Berlin, J. A.; Dulay, S.; Ang, G.; Fakharzadeh, S.; Kantor, J.; Kim, E.; Militello, G.; McGinnis, K.; Richardson, S.; Treat, J.; Vittorio, C.; Van Voorhees, A.; Werth, V. P. The CLASI (Cutaneous Lupus Erythematosus Disease Area and Severity Index): An Outcome Instrument for Cutaneous Lupus Erythematosus. J. Invest. Dermatol. 2005, 125 (5), 889-894. https://doi.org/10.1111/j.0022-202X.2005.23889.x.
(22) Yee, C.-S.; Cresswell, L.; Farewell, V.; Rahman, A.; Teh, L.-S.; Griffiths, B.; Bruce, I. N.; Ahmad, Y.; Prabu, A.; Akil, M.; McHugh, N.; D'Cruz, D.; Khamashta, M. A.; Isenberg, D. A.; Gordon, C. Numerical Scoring for the BILAG-2004 Index. Rheumatol. Oxf. Engl. 2010, 49 (9), 1665-1669. https://doi.org/10.1093/rheumatology/keq026.
(23) Yee, C.-S.; Farewell, V.; Isenberg, D. A.; Rahman, A.; Teh, L.-S.; Griffiths, B.; Bruce, I. N.; Ahmad, Y.; Prabu, A.; Akil, M.; McHugh, N.; D'Cruz, D.; Khamashta, M. A.; Maddison, P.; Gordon, C. British Isles Lupus Assessment Group 2004 Index Is Valid for Assessment of Disease Activity in Systemic Lupus Erythematosus. Arthritis Rheum. 2007, 56 (12), 4113-4119. https://doi.org/10.1002/art.23130.
(24) Hochberg, M. C. Updating the American College of Rheumatology Revised Criteria for the Classification of Systemic Lupus Erythematosus. Arthritis Rheum. 1997, 40 (9), 1725. https://doi.org/10.1002/art. 1780400928.
(25) Aringer, M.; Costenbader, K.; Daikh, D.; Brinks, R.; Mosca, M.; Ramsey-Goldman, R.; Smolen, J. S.; Wofsy, D.; Boumpas, D. T.; Kamen, D. L.; Jayne, D.; Cervera, R.; Costedoat-Chalumeau, N.; Diamond, B.; Gladman, D. D.; Hahn, B.; Hiepe, F.; Jacobsen, S.; Khanna, D.; Lerstrøm, K.; Massarotti, E.; McCune, J.; Ruiz-Irastorza, G.; Sanchez-Guerrero, J.; Schneider, M.; Urowitz, M.; Bertsias, G.; Hoyer, B. F.; Leuchten, N.; Tani, C.; Tedeschi, S. K.; Touma, Z.; Schmajuk, G.; Anic, B.; Assan, F.; Chan, T. M.; Clarke, A. E.; Crow, M. K.; Czirják, L.; Doria, A.; Graninger, W.; Halda-Kiss, B.; Hasni, S.; Izmirly, P. M.; Jung, M.; Kumanovics, G.; Mariette, X.; Padjen, I.; Pego-Reigosa, J. M.; Romero-Diaz, J.; Rua-Figueroa Fernandez, I.; Seror, R.; Stummvoll, G. H.; Tanaka, Y.; Tektonidou, M. G.; Vasconcelos, C.; Vital, E. M.; Wallace, D. J.; Yavuz, S.; Meroni, P. L.; Fritzler, M. J.; Naden, R.; Dörner, T.; Johnson, S. R. 2019 European League Against Rheumatism/American College of Rheumatology Classification Criteria for Systemic Lupus Erythematosus. Arthritis Rheumatol. Hoboken NJ 2019, 71 (9), 1400-1412. https://doi.org/10.1002/art.40930.
(26) Tummala, R.; Rouse, T.; Berglind, A.; Santiago, L. Safety, Tolerability and Pharmacokinetics of Subcutaneous and Intravenous Anifrolumab in Healthy Volunteers. Lupus Sci. Med. 2018, 5 (1), e000252. https://doi.org/10.1136/lupus-2017-000252.
(27) Tanaka, Y.; Tummala, R. Anifrolumab, a Monoclonal Antibody to the Type I Interferon Receptor Subunit 1, for the Treatment of Systemic Lupus Erythematosus: An Overview from Clinical Trials. Mod. Rheumatol. 2020, 0 (0), 1-12. https://doi.org/10.1080/14397595.2020.1812201.
(28) Daikh, D. I.; Wofsy, D. Cutting Edge: Reversal of Murine Lupus Nephritis with CTLA4lg and Cyclophosphamide. J. Immunol. 2001, 166 (5), 2913-2916. https://doi.org/10.4049/jimmunol. 166.5.2913.
(29) Merrill, J. T.; Burgos-Vargas, R.; Westhovens, R.; Chalmers, A.; D'Cruz, D.; Wallace, D. J.; Bae, S. C.; Sigal, L.; Becker, J.-C.; Kelly, S.; Raghupathi, K.; Li, T.; Peng, Y.; Kinaszczuk, M.; Nash, P. The Efficacy and Safety of Abatacept in Patients with Non-Life-Threatening Manifestations of Systemic Lupus Erythematosus: Results of a Twelve-Month, Multicenter, Exploratory, Phase IIb, Randomized, Double-Blind, Placebo-Controlled Trial. Arthritis Rheum. 2010, 62 (10), 3077-3087. https://doi.org/10.1002/art.27601.
(30) Pena-Rossi, C.; Nasonov, E.; Stanislav, M.; Yakusevich, V.; Ershova, O.; Lomareva, N.; Saunders, H.; Hill, J.; Nestorov, I. An Exploratory Dose-Escalating Study Investigating the Safety, Tolerability, Pharmacokinetics and Pharmacodynamics of Intravenous Atacicept in Patients with Systemic Lupus Erythematosus. Lupus 2009, 18 (6), 547-555. https://doi.org/10.1177/0961203309102803.
(31) Merrill, J. T.; Wallace, D. J.; Wax, S.; Kao, A.; Fraser, P. A.; Chang, P.; Isenberg, D.; ADDRESS II Investigators. Efficacy and Safety of Atacicept in Patients With Systemic Lupus Erythematosus: Results of a Twenty-Four-Week, Multicenter, Randomized, Double-Blind, Placebo-Controlled, Parallel-Arm, Phase IIb Study. Arthritis Rheumatol. Hoboken NJ 2018, 70 (2), 266-276. https://doi.org/10.1002/art.40360.
(32) Wallace, D. J.; Gordon, C.; Strand, V.; Hobbs, K.; Petri, M.; Kalunian, K.; Houssiau, F.; Tak, P. P.; Isenberg, D. A.; Kelley, L.; Kilgallen, B.; Barry, A. N.; Wegener, W. A.; Goldenberg, D. M. Efficacy and Safety of Epratuzumab in Patients with Moderate/Severe Flaring Systemic Lupus Erythematosus: Results from Two Randomized, Double-Blind, Placebo-Controlled, Multicentre Studies (ALLEVIATE) and Follow-Up. Rheumatol. Oxf. Engl. 2013, 52 (7), 1313-1322. https://doi.org/10.1093/rheumatology/ket129.
(33) Clowse, M. E. B.; Wallace, D. J.; Furie, R. A.; Petri, M. A.; Pike, M. C.; Leszczyński, P.; Neuwelt, C. M.; Hobbs, K.; Keiserman, M.; Duca, L.; Kalunian, K. C.; Galateanu, C.; Bongardt, S.; Stach, C.; Beaudot, C.; Kilgallen, B.; Gordon, C.; Batalov, A.; Bojinca, M.; Djerassi, R.; Duca, L.; Horak, P.; Kolarov, Z.; Milasiene, R.; Monova, D.; Otsa, K.; Pileckyte, M.; Popova, T.; Radulescu, F.; Rashkov, R.; Rednic, S.; Repin, M.; Stoilov, R.; Tegzova, D.; Vezikova, N.; Vitek, P.; Zainea, C.; East, F.; Baek, H.; Chen, Y.; Chiu, Y.; Cho, C.; Chou, C.; Choe, J.; Huang, C.; Kang, Y.; Kang, S.; Lai, N.; Lee, S.; Park, W.; Shim, S.; Suh, C.; Yoo, W.; Armengol, H. A.; Zapata, F. A.; Santiago, M. B.; Cavalcanti, F.; Chahade, W.; Costallat, L.; Keiserman, M.; Alcala, J. O.; Remus, C. R.; Roimicher, L.; Abu: Shakra, M.; Agarwal, V.; Agmon Levin, N.; Kadel, J.; Levy, Y.; Mevorach, D.; Paran, D.; Reitblat, T.; Rosner, I.; Shobha, V.; Sthoeger, Z.; Zisman, D.; Ayesu, K.; Berney, S.; Box, J.; Busch, H.; Buyon, J.; Carter, J.; Chi, J.; Clowse, M.; Collins, R.; Dao, K.; Diab, I.; Dikranian, A.; El. Shahawy, M.; Gaylis, N.; Grossman, J.; Halpert, E.; Huff, J.; Jarjour, W.; Kao, A.; Katz, R.; Kennedy, A.; Khan, M.; Kivitz, A.; Kohen, M.; Lawrence Ford, T.; Lawson, J.; Levesque, M.; Lowenstein, M.; Majjhoo, A.; Mcarthur, R.; McLain, D.; Merrill, J.; Murillo, A.; Neucks, S.; Niemer, G.; Noaiseh, G.; Parker, C.; Pantojas, C.; Pattanaik, D.; Petri, M.; Pickrell, P.; Reveille, J.; Roman: Miranda, A.; Rothfield, N.; Sankoorikal, A.; Sayers, M.; Singhal, A.; Snyder, A.; Striebich, C.; Vo, Q.; von Feldt, J.; Wallace, D.; Wasko, M.; Young, C.; Adelstein, S.; Hall, S.; Littlejohn, G.; Nicholls, D.; Suranyi, M.; Amoura, Z.; Bannert, B.; Behrens, F.; Perez, L. C.; Chakravarty, K.; Gonzales, F. D.; Davies, K.; Doria, A.; Emery, P.; Fernández Nebro, A.; Govoni, M.; Hachulla, E.; Hellmich, B.; Houssiau, F.; Malaise, M.; Margaux, J.; Maugars, Y.; Muñoz Fernandez, S.; Navarro, F.; Ordi. Ros, J.; Pellerito, R.; Pena Sagredo, J.; Roussou, E.; Schmidt, R. E.; Ucar Angulo, E.; Viallard, J.; Westhovens, R.; Worm, M.; Yee, C. S.; Nayiager, S.; Reuter, H.; Spargo, C.; Bazela, B.; Brzosko, M.; Chudzik, D.; Gasztonyi, B.; Geher, P.; Ionescu, R.; Jeka, S.; Kemeny, L.; Kiss, E.; Kotyla, P.; Kovacs, L.; Kovalenko, V.; Kucharz, E.; Kwiatkowska, B.; Leszczynski, P.; Levchenko, E.; Lysenko, G.; Majdan, M.; Mihailov, C.; Nalotov, S.; Nedelciu, M.; Pavel, M.; Raskina, T.; Rebrov, B.; Rezus, E.; Semen, T.; Smakotina, S.; Stanislavchuk, M.; Stanislav, M.; Szombati, I.; Szucs, G.; Udrea, G.; Zajdel, J.; Zon. Giebel, A.; Bonfiglioli, R.; Bustamante, R.; Klumb, E.; Ramirez, G. M.; Neiva, C.; Olguin, M.; Gonzaga, J. R.; Scotton, A.; Ayala, S. S.; Ximenes, A.; Sharma, R.; Srikantiah, C.; Aelion, J.; Aranow, C.; Baker, M.; Chadha, A.; Chao, J.; Chatham, W.; Chow, A.; Clay, C.; Cohen. Gadol, S.; Conaway, D.; Denburg, J.; Escalante, A.; Espinoza, L.; Fiechtner, J.; Fortin, I.; Fraser, A.; Furie, R.; Gladman, D.; Goddard, D.; Goldberg, M.; Gonzalez. Rivera, R.; Gorman, J.; Griffin, R.; Haaland, D.; Halter, D.; Hemaiden, A.; Hobbs, K.; Joshi, V.; Lim, S.; Kalunian, K.; Karpouzas, G.; Khraishi, M.; Lafyatis, R.; Lee, S.; Lidman, R.; Lue, C.; Mohan, M.; Mease, P.; Mehta, C.; Mizutani, W.; Nami, A.; Nascimento, J.; Neuwelt, C.; Pappas, J.; Pope, J.; Porges, A.; Roane, G.; Rosenberg, D.; Ross, S.; Saadeh, C.; Scoville, C.; Sherrer, Y.; Solomon, M.; Surbeck, W.; Valenzuela, G.; Waller, P.; Alten, R.; Baerwald, C.; Bienvenu, B.; Bombardieri, S.; Braun, J.; Dival, L.; Espinosa, G.; Fernandez, I. F.; Gomez: Reino, J.; Gordon, C.; Hiepe, F.; Hopkinson, N.; Isenberg, D.; Jacobi, A.; Jorgensen, C.; Guern, V. L.; Paul, C.; Pego Reigosa, J. M.; Heredia, J. R.; Rubbert. Roth, A.; Sabbadini, M.; Schroeder, J.; Schwarting, A.; Spieler, W.; Valesini, G.; Wollenhaupt, J.; Mendoza, A. Z.; Zouboulis, C. Efficacy and Safety of Epratuzumab in Moderately to Severely Active Systemic Lupus Erythematosus: Results From Two Phase III Randomized, Double Blind, Placebo. Controlled Trials. Arthritis Rheumatol. Hoboken Nj 2017, 69 (2), 362-375. https://doi.org/10.1002/art.39856.
(34) Gottenberg, J.-E.; Dörner, T.; Bootsma, H.; Devauchelle-Pensec, V.; Bowman, S. J.; Mariette, X.; Bartz, H.; Oortgiesen, M.; Shock, A.; Koetse, W.; Galateanu, C.; Bongardt, S.; Wegener, W. A.; Goldenberg, D. M.; Meno-Tetang, G.; Kosutic, G.; Gordon, C. Efficacy of Epratuzumab, an Anti-CD22 Monoclonal IgG Antibody, in Systemic Lupus Erythematosus Patients With Associated Sjogren's Syndrome: Post Hoc Analyses From the EMBODY Trials. Arthritis Rheumatol. Hoboken NJ 2018, 70 (5), 763-773. https://doi.org/10.1002/art.40425.
(35) Dörner, T.; Kaufmann, J.; Wegener, W. A.; Teoh, N.; Goldenberg, D. M.; Burmester, G. R. Initial Clinical Trial of Epratuzumab (Humanized Anti-CD22 Antibody) for Immunotherapy of Systemic Lupus Erythematosus. Arthritis Res. Ther. 2006, 8 (3), R74. https://doi.org/10.1186/ar1942.
(36) Merrill, J. T.; Neuwelt, C. M.; Wallace, D. J.; Shanahan, J. C.; Latinis, K. M.; Oates, J. C.; Utset, T. O.; Gordon, C.; Isenberg, D. A.; Hsieh, H.-J.; Zhang, D.; Brunetta, P. G. Efficacy and Safety of Rituximab in Moderately-to-Severely Active Systemic Lupus Erythematosus: The Randomized, Double-Blind, Phase II/III Systemic Lupus Erythematosus Evaluation of Rituximab Trial. Arthritis Rheum. 2010, 62 (1), 222-233. https://doi.org/10.1002/art.27233.
(37) Ryden-Aulin, M.; Boumpas, D.; Bultink, I.; Rubio, J. L. C.; Caminal-Montero, L.; Castro, A.; Ruiz, A. C.; Doria, A.; Dörner, T.; Gonzalez-Echavarri, C.; Gremese, E.; Houssiau, F. A.; Huizinga, T.; Inanç, M.; Isenberg, D.; Iuliano, A.; Jacobsen, S.; Jimenez-Alonso, J.; Kovács, L.; Mariette, X.; Mosca, M.; Nived, O.; Oristrell, J.; Ramos-Casals, M.; Rascón, J.; Ruiz-Irastorza, G.; Sáez-Comet, L.; Cervelló, G. S.; Sebastiani, G. D.; Squatrito, D.; Szücs, G.; Voskuyl, A.; Vollenhoven, R. van. Off-Label Use of Rituximab for Systemic Lupus Erythematosus in Europe. Lupus Sci. Med. 2016, 3 (1), e000163. https://doi.org/10.1136/lupus-2016-000163.
(38) Isenberg, D. A.; Petri, M.; Kalunian, K.; Tanaka, Y.; Urowitz, M. B.; Hoffman, R. W.; Morgan-Cox, M.; likuni, N.; Silk, M.; Wallace, D. J. Efficacy and Safety of Subcutaneous Tabalumab in Patients with Systemic Lupus Erythematosus: Results from ILLUMINATE-1, a 52-Week, Phase III, Multicentre, Randomised, Double-Blind, Placebo-Controlled Study. Ann. Rheum. Dis. 2016, 75 (2), 323-331. https://doi.org/10.1136/annrheumdis-2015-207653.
(39) Isenberg, D.; Merrill, J.; Hoffman, R.; Linnik, M.; Morgan-Cox, M.; Veenhuizen, M.; likuni, N.; Dickson, C.; Silk, M.; Wallace, D.; Dörner, T. OP0184 Efficacy and Safety of Tabalumab in Patients with Systemic Lupus Erythematosus (SLE): Results from 2 Phase 3, 52-Week, Multicenter, Randomized, Placebo-Controlled Trials. Ann. Rheum. Dis. 2015, 74 (Suppl 2), 141-141. https://doi.org/10.1136/annrheumdis-2015-eular.1195.
(40) Khamashta, M.; Merrill, J. T.; Werth, V. P.; Furie, R.; Kalunian, K.; Illei, G. G.; Drappa, J.; Wang, L.; Greth, W. Sifalimumab, an Anti-Interferon-a Monoclonal Antibody, in Moderate to Severe Systemic Lupus Erythematosus: A Randomised, Double-Blind, Placebo-Controlled Study. Ann. Rheum. Dis. 2016, 75 (11), 1909-1916. https://doi.org/10.1136/annrheumdis-2015-208562.
(41) Gerriets, V.; Bansal, P.; Goyal, A.; Khaddour, K. Tumor Necrosis Factor Inhibitors. In StatPearls; StatPearls Publishing: Treasure Island (FL), 2021.
(42) Ferguson, G. T.; Mansur, A. H.; Jacobs, J. S.; Hebert, J.; Clawson, C.; Tao, W.; Wu, Y.; Goldman, M. Assessment of an Accessorized Pre-Filled Syringe for Home-Administered Benralizumab in Severe Asthma. J. Asthma Allergy 2018, 11, 63-72. https://doi.org/10.2147/JAA.S157762.
(43) Klein, R. S.; Morganroth, P. A.; Werth, V. P. Cutaneous Lupus and the CLASI Instrument. Rheum. Dis. Clin. North Am. 2010, 36 (1), 33-51. https://doi.org/10.1016/j.rdc.2009.12.001.
(44) Touma, Z.; Urowitz, M.; Gladman, D. SLEDAI-2K for a 30-Day Window. Lupus 2010, 19 (1), 49-51. https://doi.org/10.1177/0961203309346505.
(45) Schwartz, N.; Rubinstein, T.; Burkly, L. C.; Collins, C. E.; Blanco, I.; Su, L.; Hojaili, B.; Mackay, M.; Aranow, C.; Stohl, W.; Rovin, B. H.; Michaelson, J. S.; Putterman, C. Urinary TWEAK as a Biomarker of Lupus Nephritis: A Multicenter Cohort Study. Arthritis Res. Ther. 2009, 11 (5), R143. https://doi.org/10.1186/ar2816.
(46) Interferon-Inducible Gene Expression Kit As a Potential Diagnostic Test for Anifrolumab: Analytical Validation for Use in Clinical Trials. ACR Meeting Abstracts.
(47) Kuhn, A.; Bonsmann, G.; Anders, H.-J.; Herzer, P.; Tenbrock, K.; Schneider, M. The Diagnosis and Treatment of Systemic Lupus Erythematosus. Dtsch. Ärztebl. Int. 2015, 112 (25), 423-432. https://doi.org/10.3238/arztebl.2015.0423.
(48) Bruce, I. N.; Nami, A.; Schwetje, E.; Pierson, M. E.; Rouse, T.; Chia, Y. L.; Kuruvilla, D.; Abreu, G.; Tummala, R.; Lindholm, C. Pharmacokinetics, Pharmacodynamics, and Safety of Subcutaneous Anifrolumab in Patients with Systemic Lupus Erythematosus, Active Skin Disease, and High Type I Interferon Gene Signature: A Multicentre, Randomised, Double-Blind, Placebo-Controlled, Phase 2 Study. Lancet Rheumatol. 2020, 0 (0). https://doi.org/10.1016/S2665-9913 (20) 30342-8.
(49) Ryman, J. T.; Meibohm, B. Pharmacokinetics of Monoclonal Antibodies. CPT Pharmacomet. Syst. Pharmacol. 2017, 6 (9), 576-588. https://doi.org/10.1002/psp4.12224.

1-19. (canceled)

20. A method of treating systemic lupus erythematous (SLE) in a subject in need thereof, the method comprising administering a pharmaceutical composition comprising anifrolumab to the subject, wherein the subject has severe SLE.

21. The method of claim 20, wherein pretreatment with the pharmaceutical composition comprising anifrolumab the subject has severe SLE.

22. The method of claim 20, wherein pretreatment with the pharmaceutical composition comprising anifrolumab the subject has a SLEDAI-2K score of ≥10.

23-32. (canceled)

33. The method of claim 20, wherein the method comprises administering the pharmaceutical composition intravenously.

34. The method of claim 20, wherein the pharmaceutical composition comprises ≥300 mg anifrolumab.

35. The method of claim 20, wherein the pharmaceutical composition comprises ≤1000 mg anifrolumab.

36. The method of claim 20, wherein the pharmaceutical composition comprises about 300 mg, about 900 mg, or about 1000 mg anifrolumab.

37. The method of claim 20, wherein the method comprises administering the pharmaceutical composition intravenously every four weeks (Q4W)

38. The method of claim 20, wherein the method comprises administering the pharmaceutical composition subcutaneously.

39. The method of claim 38, wherein the pharmaceutical composition comprises >105 mg and <150 mg anifrolumab.

40. The method of claim 38, wherein the pharmaceutical composition comprises ≤135 mg anifrolumab.

41. The method of claim 38, wherein the pharmaceutical composition comprises 120 mg anifrolumab.

42. The method of claim 38, wherein the pharmaceutical composition is administered in a single administration step.

43. The method of claim 38, wherein the pharmaceutical composition is administered at intervals of 6-8 days.

44. The method of claim 38, wherein the pharmaceutical composition is administered once per week.

45. The method of claim 38, wherein the pharmaceutical composition has a volume of about 0.5 to about 1 ml.

46. The method of claim 45, wherein the pharmaceutical composition has a volume of about 0.8 ml.

47-87. (canceled)
